SEQUENCE LISTING (49 sequences)

SEQ ID NO: 1 - protein, 32 aa, Artificial Sequence
  SYNTHESIZED
  MISC_FEATURE (5)..(5): Xaa can be Lys or Gln
  MISC_FEATURE (12)..(12): Xaa can be Gly or Asp
MAEVXLVESGGXLVKPGGSLKLSCAASGFTFS

SEQ ID NO: 2 - protein, 14 aa, Artificial Sequence
  SYNTHESIZED
  MISC_FEATURE (7)..(7): Xaa can be Asp or Glu
WVRQTPXKRLEWVA

SEQ ID NO: 3 - protein, 32 aa, Artificial Sequence
  SYNTHESIZED
  MISC_FEATURE (21)..(21): Xaa can be Arg or Lys
  MISC_FEATURE (27)..(27): Xaa can be Ile or Met
RFTISRDNAKNTLYLQMSSLXSEDTAXYYCAR

SEQ ID NO: 4 - protein, 9 aa, Artificial Sequence
  SYNTHESIZED
  MISC_FEATURE (6)..(6): Xaa can be Ser or Thr
  MISC_FEATURE (7)..(7): Xaa can be Leu or Val
WGQGTXXTV

SEQ ID NO: 5 - protein, 23 aa, Artificial Sequence
  SYNTHESIZED
  MISC_FEATURE (12)..(12): Xaa can be Ala or Thr
DIVLTQSPASLXVSLGQRATISC

SEQ ID NO: 6 - protein, 15 aa, Artificial Sequence
  SYNTHESIZED
WYQQKPGQPPKLLIY

SEQ ID NO: 7 - protein, 32 aa, Artificial Sequence
  SYNTHESIZED
GIPARFSGSGSGTDFTLNIHPVEEEDAATYYC

SEQ ID NO: 8 - protein, 9 aa, Artificial Sequence
  SYNTHESIZED
  MISC_FEATURE (3)..(3): Xaa can be Ala or Gly
FGXGTKLEI

SEQ ID NO: 9 - protein, 122 aa, Artificial Sequence
  SYNTHESIZED
MAEVKLVESGGGLVKPGGSLKLSCAASGFTFSSYAMSWVRQTPEKRLEWVATISSGGSYTYYPNTVKGRFTISRDNAKNTLYLQMSSLRSEDTAIYYCARHGVRHRVDYFDYWGQGTTLTVS

SEQ ID NO: 10 - protein, 110 aa, Artificial Sequence
  SYNTHESIZED
DIVLTQSPASLTVSLGQRATISCKASQSVDYDGDSYMNWYQQKPGQPPKLLIYAASNLESGIPARFSGSGSGTDFTLNIHPVEEEDAATYYCQQSNEDPLTFGAGTKLEI

SEQ ID NO: 11 - protein, 249 aa, Artificial Sequence
  SYNTHESIZED
MAEVKLVESGGGLVKPGGSLKLSCAASGFTFSSYGMSWVRQTPEKRLEWVATISRGGSYTYYPDSVKGRFTISRDNAKNTLYLQMSSLRSEDTAIYYCARRETYDEKGFAYWGQGTTLTVSSGGGGSGGGGSGGGGSDIVLTQSPASLTVSLGQRATISCKSSQSLLNSGNQKNYLTWYQQKPGQPPKLLIYWASTRESGIPARFSGSGSGTDFTLNIHPVEEEDAATYYCQNDNSHPLTFGAGTKLEI

SEQ ID NO: 12 - protein, 249 aa, Artificial Sequence
  SYNTHESIZED
MAEVQLVESGGDLVKPGGSLKLSCAASGFTFSSYGMSWVRQTPDKRLEWVATISRGGSYTYYPDSVKGRFTISRDNAKNTLYLQMSSLKSEDTAMYYCARRETYDEKGFAYWGQGTSVTVSSGGGGSGGGGSGGGGSDIVLTQSPASLAVSLGQRATISCKSSQSLLNSGNQKNYLTWYQQKPGQPPKLLIYWASTRESGIPARFSGSGSGTDFTLNIHPVEEEDAATYYCQNDNSHPLTFGGGTKLEI

SEQ ID NO: 13 - protein, 246 aa, Artificial Sequence
  SYNTHESIZED
MAEVKLVESGGGLVKPGGSLKLSCAASGFTFSSFGMHWVRQTPEKRLEWVAYISSGSSTIYYADTVKGRFTISRDNAKNTLYLQMSSLRSEDTAIYYCARSLATAAFAYWGQGTTLTVSSGGGGSGGGGSGGGGSDIVLTQSPASLTVSLGQRATISCRSSQSIVYSNGNTYLEWYQQKPGQPPKLLIYKVSNRFSGIPARFSGSGSGTDFTLNIHPVEEEDAATYYCFQGSHVPYTFGAGTKLEI

SEQ ID NO: 14 - protein, 246 aa, Artificial Sequence
  SYNTHESIZED
MAEVQLVESGGDLVKPGGSLKLSCAASGFTFSSFGMHWVRQTPDKRLEWVAYISSGSSTIYYADTVKGRFTISRDNAKNTLYLQMSSLKSEDTAMYYCARSLATAAFAYWGQGTSVTVSSGGGGSGGGGSGGGGSDIVLTQSPASLAVSLGQRATISCRSSQSIVYSNGNTYLEWYQQKPGQPPKLLIYKVSNRFSGIPARFSGSGSGTDFTLNIHPVEEEDAATYYCFQGSHVPYTFGGGTKLEI

SEQ ID NO: 15 - protein, 252 aa, Artificial Sequence
  SYNTHESIZED
MAEVKLVESGGGLVKPGGSLKLSCAASGFTFSRFGMHWVRQTPEKRLEWVAYISSGSSTIYYADTVKGRFTISRDNAKNTLYLQMSSLRSEDTAIYYCARSGGIERYDGTYYVMDYWGQGTTLTVSSGGGGSGGGGSGGGGSDIVLTQSPASLTVSLGQRATISCRASESVDYYGKSFMNWYQQKPGQPPKLLIYAASNQGSGIPARFSGSGSGTDFTLNIHPVEEEDAATYYCQQSKEVPWTFGAGTKLEI

SEQ ID NO: 16 - protein, 252 aa, Artificial Sequence
  SYNTHESIZED
MAEVQLVESGGDLVKPGGSLKLSCAASGFTFSRFGMHWVRQTPDKRLEWVAYISSGSSTIYYADTVKGRFTISRDNAKNTLYLQMSSLKSEDTAMYYCARSGGIERYDGTYYVMDYWGQGTSVTVSSGGGGSGGGGSGGGGSDIVLTQSPASLAVSLGQRATISCRASESVDYYGKSFMNWYQQKPGQPPKLLIYAASNQGSGIPARFSGSGSGTDFTLNIHPVEEEDAATYYCQQSKEVPWTFGGGTKLEI

SEQ ID NO: 17 - protein, 238 aa, Artificial Sequence
  SYNTHESIZED
MAEVKLVESGGGLVKPGGSLKLSCAASGFTFSSYAMSWVRQTPEKRLEWVAAISGRGYNADYADSVKGRFTISRDNAKNTLYLQMSSLRSEDTAIYYCARLEYFDYWGQGTTLTVSSGGGGSGGGGSGGGGSDIVLTQSPASLTVSLGQRATISCRASQSISSWLAWYQQKPGQPPKLLIYKASSLESGIPARFSGSGSGTDFTLNIHPVEEEDAATYYCQQYNSYPLTFGAGTKLEI

SEQ ID NO: 18 - protein, 5 aa, Artificial Sequence
  SYNTHESIZED
GGGGS

SEQ ID NO: 19 - protein, 15 aa, Artificial Sequence
  SYNTHESIZED
GGGGSGGGGSGGGGS

SEQ ID NO: 20 - protein, 17 aa, Artificial Sequence
  SYNTHESIZED
SSGGGGSGGGGSGGGGS

SEQ ID NO: 21 - protein, 11 aa, Artificial Sequence
  SYNTHESIZED
MSLPGRWKPKM

SEQ ID NO: 22 - protein, 9 aa, Artificial Sequence
  SYNTHESIZED
LPGRWKPKM

SEQ ID NO: 23 - protein, 9 aa, Artificial Sequence
  SYNTHESIZED
YPYDVPDYA

SEQ ID NO: 24 - protein, 19 aa, Artificial Sequence
  SYNTHESIZED
EELLSKNYHLENEVARLKK

SEQ ID NO: 25 - protein, 32 aa, Artificial Sequence
  SYNTHESIZED
MAEVKLVESGGGLVKPGGSLKLSCAASGFTFS

SEQ ID NO: 26 - protein, 14 aa, Artificial Sequence
  SYNTHESIZED
WVRQTPEKRLEWVA

SEQ ID NO: 27 - protein, 32 aa, Artificial Sequence
  SYNTHESIZED
RFTISRDNAKNTLYLQMSSLRSEDTAIYYCAR

SEQ ID NO: 28 - protein, 9 aa, Artificial Sequence
  SYNTHESIZED
WGQGTTLTV

SEQ ID NO: 29 - protein, 23 aa, Artificial Sequence
  SYNTHESIZED
DIVLTQSPASLTVSLGQRATISC

SEQ ID NO: 30 - protein, 15 aa, Artificial Sequence
  SYNTHESIZED
WYQQKPGQPPKLLIY

SEQ ID NO: 31 - protein, 32 aa, Artificial Sequence; identical to SEQ ID NO: 7
  SYNTHESIZED

SEQ ID NO: 32 - protein, 9 aa, Artificial Sequence
  SYNTHESIZED
FGAGTKLEI

SEQ ID NO: 33 - protein, 238 aa, Artificial Sequence
  SYNTHESIZED
MAEVKQVESGGDLVKPGGSLKLSCAASGFTFSSYAMSWVRQTPDKRLEWVAAISGRGYNADYADSVKGRFTISRDNAKNTLYLQMSSLKSEDTAMYYCARLEYFDYWGQGTSVTVSSGGGGSGGGGSGGGGSDIVLTQSPASLAVSLGQRATISCRASQSISSWLAWYQQKPGQPPKLLIYKASSLESGIPARFSGSGSGTDFTLNIHPVEEEDAATYYCQQYNSYPLTFGGGTKLEI

SEQ ID NO: 34 - protein, 7 aa, Artificial Sequence
  SYNTHESIZED
PKKKRKV

SEQ ID NO: 35 - protein, 7 aa, Artificial Sequence
  SYNTHESIZED
PKKKRRV

SEQ ID NO: 36 - protein, 16 aa, Artificial Sequence
  SYNTHESIZED
KRPAATKKAGQAKKKK

SEQ ID NO: 37 - protein, 20 aa, Artificial Sequence
  SYNTHESIZED
GRKKRRQRRRPPQPKKKRKV

SEQ ID NO: 38 - protein, 19 aa, Artificial Sequence
  SYNTHESIZED
PLSSIFSRIGDPPKKKRKV

SEQ ID NO: 39 - protein, 24 aa, Artificial Sequence
  SYNTHESIZED
GALFLGWLGAAGSTMGAPKKKRKV

SEQ ID NO: 40 - protein, 27 aa, Artificial Sequence
  SYNTHESIZED
GALFLGFLGAAGSTMGAWSQPKKKRKV

SEQ ID NO: 41 - protein, 21 aa, Artificial Sequence
  SYNTHESIZED
KETWWETWWTEWSQPKKKRKV

SEQ ID NO: 42 - protein, 5 aa, Artificial Sequence
  SYNTHESIZED
SFGMH

SEQ ID NO: 43 - protein, 17 aa, Artificial Sequence
  SYNTHESIZED
YISSGSSTIYYADTVKG

SEQ ID NO: 44 - protein, 9 aa, Artificial Sequence
  SYNTHESIZED
SLATAAFAY

SEQ ID NO: 45 - protein, 16 aa, Artificial Sequence
  SYNTHESIZED
RSSQSIVYSNGNTYLE

SEQ ID NO: 46 - protein, 7 aa, Artificial Sequence
  SYNTHESIZED
KVSNRFS

SEQ ID NO: 47 - protein, 9 aa, Artificial Sequence
  SYNTHESIZED
FQGSHVPYT

SEQ ID NO: 48 - protein, 10 aa, Artificial Sequence
  SYNTHESIZED
FAVANGNELL

SEQ ID NO: 49 - protein, 8 aa, Artificial Sequence
  SYNTHESIZED
DYKDDDDK

(19) United States
(12) Patent Application Publication
(10) Pub. No.: US 2020/0102373 A1
(43) Pub. Date: Apr. 2, 2020

(54) SCFV'S FOR LIVE CELL IMAGING AND OTHER USES

(71) Applicants: COLORADO STATE UNIVERSITY RESEARCH FOUNDATION, Fort Collins, CO (US); TOKYO INSTITUTE OF TECHNOLOGY, Tokyo (JP)

(72) Inventors: Timothy Stasevich, Fort Collins, CO (US); Ning Zhao, Fort Collins, CO (US); Hiroshi Kimura, Tokyo (JP); Yuko Sato, Tokyo (JP)

(73) Assignees: COLORADO STATE UNIVERSITY RESEARCH FOUNDATION, Fort Collins, CO (US); TOKYO INSTITUTE OF TECHNOLOGY, Tokyo (JP)

(21) Appl. No.: 16/586,085

(22) Filed: Sep. 27, 2019

Related U.S. Application Data
(60) Provisional application No. 62/737,741, filed on Sep. 27, 2018.
(60) Provisional application No. 62/826,395, filed on Mar. 29, 2019.

(51) Int. Cl. C07K 16/10; C12N 15/85; G01N 33/569

(52) U.S. Cl. CPC C07K 16/1018; C12N 15/85; G01N 33/56983; C07K 2317/565; C07K 2319/60; C07K 2317/622; C07K 2317/24; C07K 2317/92; C07K 2317/56

(57) ABSTRACT

Disclosed herein are methods to engineer single chain variable fragments (scFv's) that specifically bind a plurality of antigens in vivo, as well as novel scFv's produced therefrom. The disclosed novel scFV's can be used a variety of applications.

### CROSS REFERENCE TO RELATED APPLICATIONS

This application claims priority to U.S. Provisional Application No. 62/737,741, filed Sep. 27, 2018, and U.S. Provisional Application No. 62/826,395, filed Mar. 29, 2019, the disclosures of which are incorporated herein by reference.

### GOVERNMENT SUPPORT CLAUSE

This invention was made with government support under GM119728 awarded by the National Institutes of Health. The government has certain rights in the invention.

### REFERENCE TO SEQUENCE LISTING

This application contains a Sequence Listing that has been submitted in ASCII format via EFS-Web and is hereby incorporated by reference in its entirety. The ASCII copy, created on Sep. 25, 2019, is named “065620-636576_ST25.txt, and is 33 KB in size.

### BACKGROUND OF THE INVENTION

Live-cell imaging is critical for tracking the dynamics of cell signaling. The discovery and development of the green fluorescent protein (GFP), for example, has revolutionized the field of cell biology. GFP can be genetically fused to a protein of interest (POI) to track its expression and localization in vivo. While powerful, GFP-tagging has limitations to image the full lifecycles of proteins. First, long fluorophore maturation times prevent co-translational imaging of GFP-tagged nascent peptide chains. By the time the GFP tag folds, matures and lights up, translation is over. Similarly, slow GFP maturation times have made it difficult to image short-lived transcription factors critical for development and embryogenesis. Again, before the GFP tag lights up, the transcription factor may already be degraded. Second, GFP tags cannot discriminate post-translational modifications (PTM) of proteins, nor can they discriminate protein conformational changes. Without the ability to directly image these important protein subpopulations, their functionality is difficult to quantify and assess. Third, GFP tags are large, permanently attached, and dim. It is therefore difficult to detect and/or amplify fluorescence signal. This limits the length of time a single tagged protein can be tracked in a living cell before the protein is photobleached or the cell is photodamaged.

To address these limitations of GFP, an alternative live-cell imaging methodology has emerged that uses antibody-based probes. In this methodology, probes built from antibodies, such as antigen binding fragments (Fab's), single-chain variable fragments (scFv's), and camelid nanobodies, are conjugated or genetically fused with mature fluorophores. When expressed or loaded into cells, the probes bind and light up epitopes within POIs as soon as the epitopes are accessible. With this methodology, it is possible to visualize and quantify the co-translational dynamics of nascent peptide chains, capture the dynamics of short-lived transcription factors⁵, track single molecules for extended periods of time, and selectively track the spatiotemporal dynamics of PTMs and protein conformational changes.

While there is potential for antibody-based probes in live-cell imaging, so far only a handful have been developed. Fab's are straight-forward to develop since they can be digested from commercial antibodies and conjugated with dyes using kits. Unfortunately, Fab's have not been widely adopted because they are difficult to load into living systems. While adherent cells (e.g. U2OS) can be loaded in mass, sensitive cell types (e.g. neurons) have proven refractive to most loading procedures. Additionally, Fab are expensive, typically requiring milligrams of antibody to start with. Fab may also change considerably from batch to batch, which can lead to unwanted variability between experiments.

Given the drawbacks of Fab, genetically encoded probes are an attractive alternative. Since these probes can be integrated into plasmids, they can be distributed and cell lines and/or transgenic organisms can be generated that stably express the probes, all without batch-to-batch variability. A downside of genetically encoded antibody-based probes is they are not straightforward to develop. Both scFvs and nanobodies require a large initial investment, as either existing hybridomas or immunized animals are necessary to get the antibody sequences. Worse, even after sequences are determined, there is a good chance that antibody-based probes derived from the sequences will not fold and function properly in vivo. The problem is antibodies have evolved to be secreted from cells, so their folding and maturation is often disrupted when expressed within the reduced intracellular environment. Thus, protein engineering, directed evolution, and mutagenesis are typically needed to generate an ideal antibody-based probe that functions in vivo.

A case in point is the SunTag scFv, the only genetically encoded antibody-based probe capable of binding a small epitope co-translationally in living cells. The SunTag scFv binds a 19 aa epitope (SEQ ID NO: 24) that is repeated 24 times within a single SunTag. As multiple scFvs bind the SunTag co-translationally, fluorescence signal from tagged POIs can be amplified, enabling both single mRNA translation imaging and long-term single molecule tracking in vivo. The SunTag technology was developed over many years, starting with the Plückthun lab in 1998. The original version was evolved through directed evolution and extensive protein engineering and later tested in 2014 to stain mitochondria in living cells. The probe was further optimized via the addition of stabilizing sfGFP and GB1 domains to eliminate aggregation at higher expression levels and the original epitope was optimized to version 4 via directed mutagenesis.

The large amount of work required to develop the SunTag highlights the difficulty of generating scFv probes suitable for live-cell imaging. Accordingly, there remains a need in the art for improved methods.

### SUMMARY OF THE INVENTION

One aspect of the present disclosure encompasses a plurality of single chain variable fragments (scFv's, or each an scFv) that function (e.g., specifically bind their antigen) in reducing compartment(s) of a cell. Each scFv is comprised of a heavy chain variable region (VH), a light chain variable region (VL) and a linker connecting the VH and the VL, and has VH and VL with substantially similar framework regions as the corresponding framework regions from 15F11 but different antigen binding specificity than 15F11. The linker may or may not be a peptide linker.

In another aspect, the present disclosure encompasses a single chain variant fragment (scFv) comprising a heavy chain variable domain (VH) of Formula (I), a light chain variable domain (VL) of Formula (I), and a linker connecting VH and VL, wherein the amino acid sequence of FR1, FR2, FR3 and FR4, collectively, for the VH has at least 80% identity to the amino acid sequence of the framework regions of SEQ ID NO: 9; the amino acid sequence of FR1, FR2, FR3 and FR4, collectively, for the VL has at least 65% identity to the amino acid sequence of the framework regions of SEQ ID NO: 10; and the scFv has a different antigen-binding specificity than 15F11. The VH of the scFV may also have at least about 74% identity to SEQ ID NO: 9 and/or the VL of the scFV has at least about 63% identity to SEQ ID NO: 10. In preferred embodiments, the linker is a peptide linker. In some embodiments, the peptide linker comprises (GGGGS)ₙ, wherein n is 1 to 6. Also contemplated are proteins comprising each scFv, as well as polynucleotides encoding the scFv or the protein.

In another aspect, the present disclosure encompasses a single chain variant fragment (scFv) comprising a heavy chain variable domain (VH) of Formula (I), a light chain variable domain (VL) of Formula (I), and a linker connecting VH and VL, wherein the amino acid sequence of FR1, FR2, FR3 and FR4, collectively, for the VH has at least 84% identity to the amino acid sequence of the framework regions of SEQ ID NO: 9; the amino acid sequence of FR1, FR2, FR3 and FR4, collectively, for the VL has at least 69% identity to the amino acid sequence of the framework regions of SEQ ID NO: 10; and the scFv has a different antigen-binding specificity than 15F11. The VH of the scFV may also have at least about 74% identity to SEQ ID NO: 9 and/or the VL of the scFV has at least about 63% identity to SEQ ID NO: 10. In preferred embodiments, the linker is a peptide linker. In some embodiments, the peptide linker comprises (GGGGS)ₙ, wherein n is 1 to 6. Also contemplated are proteins comprising each scFv, as well as polynucleotides encoding the scFv or the protein.

In another aspect, the present disclosure encompasses a method for live cell imaging, the method comprising providing a protein comprising an scFv of the present disclosure linked to a detectable signal, and a cell comprising an epitope to which the scFv specifically binds; labeling the cell with the protein; and imaging the cell to detect and optionally quantify the protein.

Other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1A is a schematic showing how to design a chimeric anti-HA scFv using 12CA5-scFv CDRs and stable scFv scaffolds.

FIG. 1B is an illustration showing one embodiment for screening chimeric anti-HA scFvs in living cells. The target protein (e.g., H2B), the reporter (mCherry on the target protein and GFP on the scFv), and the number of epitope tags (e.g., HA tag) can vary.

FIG. 1C are micrographs from initial screening experiments showing the respective localization of the five chimeric anti-HA scFvs in living U2OS cells co-expressing HA-tagged histone H2B (chimeric anti-HA scFv, green; 4×HA-mCh-H2B, magenta). From left to right, n=17, 33, 27, 31, 25 cells. All images are representative cell images from one independent experiment. Scale bars: 10 μm.

FIG. 1D are micrographs showing the respective localization of χ_{15F11}^{HA} and χ_{2E2}^{HA} in living cells lacking HA-tagged histone H2B (chimeric anti-HA scFv, green; mCh-H2B, magenta). From left to right, n=21,15 cells. All images are representative cell images from one independent experiment. Scale bars: 10 μm.

FIG. 1E is a graph showing nuclear to cytoplasmic fluorescent intensity ratio (Nuc/Cyt) of each chimeric anti-HA scFv for all cells imaged as in FIG. 1C and FIG. 1D. Student's t-test. ****p<0.0001. For the box and whisker plots, median is shown by a white line, the box indicates 25-75% range, and whiskers indicate 5-95% range.

FIG. 2A contains representative images showing Frankenbody (FB-GFP; green) labels a 1×HA-tagged nuclear protein, histone H2B (magenta), at the N- or C-terminus in living U2OS cells. Top: 1×HA at C-terminus (H2B-mCh-1×HA, n=27 cells); Bottom: 1×HA at N-terminus (1×HA-mCh-H2B, n=27 cells). All images are a representative cell image of the total number of cells in one independent experiment. Scale bars, 10 μm.

FIG. 2B is a graph showing nuclear to cytoplasmic fluorescent intensity ratio (Nuc/Cyt) plot of all cells imaged as in FIG. 2A. For the box and whisker plots, median is shown by a white line, the box indicates 25-75% range, and whiskers indicate 5-95% range.

FIG. 2C contains representative images showing FB-GFP (green) labels HA-tagged cytoplasmic protein β-actin (4×HA-mCh-β-actin, magenta, n=18) in living U2OS cells and membrane protein Kv2.1 (4×HA-mRuby-Kv2.1, magenta, n=13) in living neurons. All images are a representative cell image of the total number of cells in one independent experiment. Scale bars, 10 μm.

FIG. 2D contains representative images showing FB-GFP (green) labels 1×HA or 10×HA spaghetti monster (smHA) tagged mitochondrial protein mitoNEET (Mito, magenta) in living U2OS cells. Left: Mito-mCh-1×HA (n=24); right: Mito-mCh-smHA (n=19). All images are a representative cell image of the total number of cells in one independent experiment. Scale bars, 10 μm.

FIG. 2E is a graph showing mitochondria to background fluorescent intensity ratio (Mito/Bg) plot of all cells imaged as in FIG. 2D. Mean of Mito/Bg=3.5±0.2 (Mean±SEM) for 1×HA and 16.6±1.5 (Mean±SEM) for smHA. This result shows the Mito/Bg ratio is 4.7±0.5 (SEM) times higher for smHA tagged Mito than 1×HA tagged Mito. Student's t-test. ****p<0.0001. For the box and whisker plots, median is shown by a white line, the box indicates 25-75% range, and whiskers indicate 5-95% range.

FIG. 2F contains representative images showing Frankenbody (FB) fused to multiple fluorescent fusion proteins specifically labels HA-tagged nuclear protein H2B (smHA-H2B). Top row: GFP, HaloTag-JF646, SNAP-tag-JF646 and mCherry; From left to right, n=18, 28, 23, 26 cells. In all cases, cells lacking the HA-tag display relatively even FB fluorescence. Bottom row: from left to right, n=21, 19, 11, 14 cells. See also FIG. 11. All images are a representative cell image of the total number of cells one independent experiment. Scale bars, 10 μm.

FIG. 2G is a graph showing nuclear to cytoplasmic fluorescent intensity ratio (Nuc/Cyt) plot of all cells imaged as in the top row of FIG. 2F. Student's t-test. ****p<0.0001. For the box and whisker plots, median is shown by a white line, the box indicates 25-75% range, and whiskers indicate 5-95% range.

FIG. 3A shows representative images following immunostaining in fixed U2OS cells with purified frankenbody (FB-GFP; green) of an HA-tagged nuclear protein, histone H2B (4×HA-mCh-H2B; magenta). This is a representative cell image of n=27 cells in one independent experiment. Scale bars, 10 μm.

FIG. 3B shows representative images following immunostaining in fixed U2OS cells with purified frankenbody (FB-GFP; green) of an HA-tagged cytoplasmic protein, β-actin (4×HA-mCh-β-actin; magenta). This is a representative cell image of n=10 cells in one independent experiment). Scale bars, 10 μm.

FIG. 3C is an image of a Western blot of HA-tagged H2B and β-actin. Left: purified FB-GFP (1:2000 dilution, no secondary antibody) detected directly using GFP fluorescence; Right: parental anti-HA antibody 12CA5 (1:2000 dilution) detected with secondary anti-mouse antibody/Alexa488 (1:5000 dilution).

FIG. 4A contains images from a representative FRAP experiment (yellow circle indicates bleach spot) showing fluorescence recovery in cells expressing frankenbody (FB-GFP; green) and target 4×HA-mCh-H2B (magenta). Scale bars, 10 μm.

FIG. 4B is a graph showing quantification of FRAP data in a representative cell, along with a fitted curve.

FIG. 4C contains images from a representative FRAP experiment (yellow circle indicates bleach spot) in cells expressing FB-GFP only (i.e. cells lacking HA-tags) is complete in less than 10 seconds. Student's t-test. ****p<0.0001. Scale bars, 10 μm.

FIG. 4D is a graph showing half recovery time of FRAP experiments (FB-GFP and 4×HA-mCh-H2B, n=12 cells in 2 independent experiments, as in FIG. 4B) and controls (FB-GFP only, n=8 cells in one independent experiment, as in FIG. 4C). Fits from 12 cells reveal the FRAP mean half recovery time, t_{half}, is 141±7 sec (cell-to-cell SEM). For the box and whisker plots, median is shown by a white line, the box indicates 25-75% range, and whiskers indicate 5-95% range.

FIG. 5A are images of frankenbody (FB) bound to 1×HA-H2B in a cell. The mean positions of tracks of single frankenbody (FB) bound to 1×HA-H2B provides a mobility map of H2B across the cell nucleus (10,949 tracks were generated from 977,516 total FB localizations). Tracks are color coded according to their average frame-to-frame jump size. The lighter colored tracks with relatively small jump sizes are enriched along the edge of the cell nucleus, where heterochromatin is typically enriched. To ensure tracks represent FB bound to HA-H2B, tracks were filtered such that their length had to be at least 16 consecutive frames and jumps between frames had to all be less than 220 nm. Full tracks within the yellow box are displayed in the zoom-in on the right. Scale bar, 5 μm.

FIG. 5B is a graph showing that in cells expressing HA-H2B, the number of filtered FB tracks were between one and two orders of magnitude greater than in control cells lacking HA-H2B, demonstrating false-positive tracks are rare (see FIG. 14). For the box and whisker plots, median is shown by a white line, the box indicates 25-75% range, and whiskers indicate 5-95% range.

FIG. 5C is a graph showing that the average mean-squared displacement of tracks provides a good estimate for average HA-H2B mobility. All tracks are from n=9 cells in 3 independent experiments. Student's t-test. Error bar, cell-to-cell SD of the average MSD. For the box and whisker plots, median is shown by a white line, the box indicates 25-75% range, and whiskers indicate 5-95% range.

FIG. 6A is an illustration of one embodiment where frankenbody (FB-GFP; green) and MCP-HaloTag-JF646 (magenta) label HA epitopes and mRNA stem loops, respectively, in a KDM5B translation reporter.

FIG. 6B is an image of a representative cell (10 cells in 3 independent experiments) showing colocalization of FB-GFP (green) with KDM5B mRNA (magenta).

FIG. 6C is an image of a representative cell (upper-left, 9 cells in 3 independent experiments) showing the disappearance of nascent chain spots labeled by FB-GFP within seconds of adding the translational inhibitor puromycin. Upper-right: The mean number of nascent chain spots normalized to pre-puromycin levels decreases while mRNA levels remain constant (9 cells from 3 independent experiments). Error bars, cell-to-cell SEM. Lower: a sample single mRNA montage.

FIG. 6D is an illustration of one embodiment where FB-Halo-JF646, FB-mCh, or FB-SNAP-JF646 labeling HA epitopes in a KDM5B translation reporter.

FIG. 6E is an image of representative cells (3 cells in 2 independent experiments for both FB-Halo and FB-mCh), single mRNA montages, and quantification, as in FIG. 6C, showing the loss of nascent chain spots labeled by FB-Halo-JF646 or (f) FB-mCh upon puromycin treatment. Scale bars, 10 μm. Source data are provided as a Source Data file.

FIG. 6F is an image of representative cells (3 cells in 2 independent experiments for both FB-Halo and FB-mCh), single mRNA montages, and quantification, as in FIG. 6C, showing the loss of nascent chain spots labeled by FB-mCh upon puromycin treatment. Scale bars, 10 μm.

FIG. 7A is an illustration of one embodiment where frankenbody (FB-mCh) and Sun-GFP label epitopes in the KDM5B and kif18b translation reporter constructs, respectively.

FIG. 7B is an image of a representative living U2OS cell (n=8 cells from 3 independent experiments) showing nascent chain translation spots labeled by FB-mCh (magenta) or Sun-GFP (green).

FIG. 7C depicts the mask and tracks of the cell in FIG. 7B, and dynamics of a representative translation spot for each probe (Sun-GFP, green; FB-mCh, magenta). Right, the average mean squared displacement of FB-mCh (magenta) and Sun-GFP (green) translation sites (upper-right) from N=401 tracks for FB-mCh, N=285 tracks for Sun-GFP in 8 cells in 3 independent experiments. Error bars, RNA-to-RNA SEM. Fits to the first five points of the MSD curves show the diffusion coefficients are: 0.016±0.004 μm² per sec (95% CI) for FB-mCh and 0.019±0.006 μm² per sec (95% CI) for Sun-GFP. Scale bars, 10 μm.

FIG. 8A is an illustration depicting the preparation of rat primary cortical neurons for imaging.

FIG. 8B is an image of the dendrite of a sample living neuron expressing frankenbody (FB-GFP) and the smHA-KDM5B-MS2 translation reporter (n=15 cells in 2 independent experiments). White arrows indicate translation sites that were tracked, as depicted in the illustration on the left. The spatiotemporal evolution of one mRNA track with directed motion is shown through time. Scale bars, 10 μm.

FIG. 8C is an image of a representative cell following puromycin treatment. Two circled translation sites were tracked as they disappeared following the addition of puromycin. Upper-right: The mean number of nascent chain spots normalized to pre-puromycin levels decreases after puromycin treatment (N=48 spots from 3 cells in 3 independent experiments). Error bars, cell-to-cell SEM. Scale bars, 10 μm.

FIG. 8D is a graph depicting the travel distance through time for the translation spot highlighted in FIG. 8B. Gray highlights in FIG. 8D and black arrows in FIG. 8B indicate directed motion events.

FIG. 8E is a plot of velocities faster than 1 μm per sec (37 velocities from 8 cells in 2 independent experiments). Mean: 1.40±0.07 mm per sec (spot-to-spot SEM). See also FIG. 15. Scale bars, 10 μm. For the box and whisker plots, median is shown by a white line, the box indicates 25-75% range, and whiskers indicate 5-95% range.

FIG. 9A contains max-projection images from a zebrafish embryo with frankenbody (FB-GFP) and 4×HA-mCh-H2B. Cy5-Fab labels nuclear histone acetylation as a positive control. Scale bar, 50 mm.

FIG. 9B is a graph showing a nuclei (dash circle in FIG. 9A) and its progeny tracked in development. The nuclear to cytoplasmic ratio (Nuc/Cyt) from FB-GFP (green circles), HA-H2B-mCh (magenta squares) and Fab (gray diamonds) in the parental nuclei (dotted line). Scale bar, 50 mm.

FIG. 9C contains graphs showing cell count (top), average nuclear area (units of pixel² with one pixel=662 nm, middle) and Nuc/Cyt ratios (bottom) for all tracked nuclei. See also FIG. 16 and FIG. 17. 2 embryos in 2 independent experiments. Error bars, SEM.

FIG. 10 is a sequence alignment between χ_{15F11}^{HA} (SEQ ID NO: 11) and χ_{2E2}^{HA}(SEQ ID NO: 12). The six CDRs are highlighted in yellow. Mismatched amino acids are highlighted in green.

FIG. 11A is a representative cell image of FB-GFP binding to smHA-Kv2.1 (n=2 cells in one independent experiment). Scale bars, 10 μm.

FIG. 11B is a representative image of a cell co-expressing FB-GFP (green) with other FB fused to different fluorescent proteins, and binding of the frakenbodies to smHA-H2B. From top to bottom: FB-GFP+FB-mCh (n=20), FB-GFP+FB-Halo-JF646 (n=20), FB-GFP+FB-SNAP-JF646 (n=20) in one independent experiment. Scale bars, 10 μm.

FIG. 12A is an image of an uncropped and unprocessed Western blots of HA-tagged H2B and β-actin. Purified FB-GFP (1:2000 dilution, no secondary antibody) detected directly using GFP fluorescence;

FIG. 12B is an image of an uncropped and unprocessed Western blots of HA-tagged H2B and β-actin. Parental anti-HA antibody 12CA5 (1:2000 dilution) detected with secondary anti-mouse antibody/Alexa488 (1:5000 dilution).

FIG. 13 is a graph showing the binding kinetics of frankenbody to HA tag in vitro. K_{D}=14.7±7.4 nM (Mean±SEM). Two independent experiments.

FIG. 14A has images showing all single molecule tracks of Halo-tagged frankenbody (FB) in 3 independent experiments. To increase the density of tracks within cells, the TMR-Halo ligand was pretreated with 50 mM sodium borohydride prior to staining. Tracks are color coded according to their time of acquisition (lighter is later during the movie). To ensure tracks represent FB bound to HA-H2B, a filter was used. The filter eliminated tracks of length less than 16 frames. Further, all jumps between frames had to be less than 220 nm. The mean track length is 38±6 frames (mean±SD). The number of tracks is shown at the bottom of each image. Scale bar, 5 mm.

FIG. 14B is similar to FIG. 14A, but in control cells loaded with FB but lacking HA epitopes in one independent experiment. Scale bar, 5 mm.

FIG. 15A depicts a representative translation spot in a neuron showing motored movement along neuron dendrites. Top: movement of the motored translation spot through time; bottom, velocity change through time. Sharp peaks indicate motored movement.

FIG. 15B depicts a representative translation spot, as in FIG. 15A, but in a different neuron.

FIG. 16A is an illustration depicting the timing of zebrafish embryo imaging experiments. Embryos were loaded with HA frankenbody (FB-GFP) and N×HA-mCh-H2B (N=1,4,10) (absent in control).

FIG. 16B has sample max-projection images from a control zebrafish embryo with FB-GFP (green), but lacking target HA-mCh-H2B (Empty; magenta). Positive control Cy5-Fab marks histone acetylation in nuclei. Scale bar: 50 μm.

FIG. 16C graphically depicts cell count (top), average nuclear area (units of pixel² with one pixel=662 nm, middle) and nuclear to cytoplasmic (Nuc/Cyt) ratio for all tracked nuclei (bottom). FIG. 17 (left column) shows a repeat control. Error bars, SEM.

FIG. 17 graphically depicts FB-GFP signal improves with more HA epitopes present in Zebrafish embryos. Zebrafish embryos were injected with mRNA encoding frankenbody (FB-GFP) and N×HA-mCh-H2B (from left to right, N=0,1,4,10). Embryos were also injected with Cy5-Fab to mark histone acetylation in the nuclei. Cell count (top), average nuclear area (middle) and nuclear to cytoplasmic (Nuc/Cyt) ratio in all tracked cells through time. With N=1, the green FB-GFP curve nuclear to cytoplasm ratio is consistently lower than the magenta 1×HA-mCh-H2B. With N=4, the ratios are more equal. With N=10, the green nuclear to cytoplasm ratio is consistently higher than the magenta 10×HA-mCh-H2B. One zebrafish embryo in one independent experiment for each construct. Error bars, SEM between cells.

FIG. 18A is an illustration showing how to design a chimeric anti-FLAG scFv using wtFLAG-scFv CDRs and stable scFv scaffolds.

FIG. 18B shows a sequence identity analysis of the wtFLAG-scFv with five intracellular scFv scaffolds.

FIG. 18C is an illustration showing one embodiment for screening chimeric anti-FLAG scFvs in living cells. The target protein (e.g., H2B), the reporter (mCherry on the target protein and GFP on the scFv), and the number of epitope tags (e.g., FLAG tag) can vary.

FIG. 18D is a representative cell showing the respective localization of the wildtype anti-FLAG-scFv in living U2OS cells co-expressing FLAG-tagged histone H2B (wtFLAG-scFv, green; 4×FLAG-mCh-H2B, magenta).

FIG. 18E are images from the initial screening showing the respective localization of the five chimeric anti-FLAG scFvs in living U2OS cells co-expressing FLAG-tagged histone H2B (chimeric anti-FLAG scFv, green; 4×FLAG-mCh-H2B, magenta). Scale bars: 10 μm.

FIG. 18F are images from the control results of FIG. 18E showing the respective localization of χ_{15F11}^{FLAG} and χ_{2E2}^{FLAG} in living cells lacking FLAG-tagged histone H2B (chimeric anti-FLAG scFv, green; mCh-H2B, magenta). Scale bars: 10 μm.

FIG. 19A are images showing anti-FLAG Frankenbody (anti-FLAG-FB-GFP; green) labels an FLAG-tagged cytoplasm protein, β-actin (4×FLAG-mCh-β-actin; magenta), in living U2OS cells.

FIG. 19B are images showing FB fused to multiple fluorescent fusion proteins (GFP, HaloTag-JF646, SNAP-tag-JF646 and mCherry) specifically labels FLAG-tagged nuclear protein H2B (FLAG-tagged H2B). Scale bars, 10 μm.

FIG. 20A is a diagram depicting frankenbody (anti-FLAG-FB-GFP; green) and MCP-HaloTag-JF646 (magenta) labeling FLAG epitopes and mRNA stem loops, respectively, in a KDM5B translation reporter.

FIG. 20B is a representative cell showing colocalization of anti-FLAG-FB-GFP (green) with KDM5B mRNA (magenta). Scale bars, 10 μm.

FIG. 20C is a representative translation spot (highlighted in FIG. 19B) montage showing the disappearance of nascent chain spots labeled by anti-FLAG-FB-GFP within seconds of adding the translational inhibitor puromycin.

FIG. 21A is an illustration showing how to design a chimeric anti-HIV protease scFv using wildtype scFv (wtHIV-scFv) CDRs and stable scFv scaffolds.

FIG. 21B shows a sequence identity analysis of the wtHIV-scFv with five intracellular scFv scaffolds.

FIG. 21C depicts the published binding epitope tag sequence of the anti-HIV protease scFv.

FIG. 21D is an illustration showing one embodiment for screening chimeric anti-HIV scFvs in living cells. The target protein (e.g., H2B), the reporter (mCherry on the target protein and GFP on the scFv), and the number of epitope tags (e.g., HIV tag) can vary.

FIG. 21E has images of a representative cell showing the respective localization of the wildtype anti-HIV protease scFv in living U2OS cells co-expressing the cognate HIV protease epitope-tagged histone H2B (wtHIV-scFv, green; 4×HIV-mCh-H2B, magenta). Scale bars: 10 μm.

FIG. 21F has images of initial screening results showing the respective localization of 2 good chimeric anti-HIV scFvs in living U2OS cells co-expressing HIV protease epitope-tagged histone H2B (chimeric anti-HIV scFv, green; 4×HIV-mCh-H2B, magenta). Scale bars: 10 μm.

FIG. 21G are images of controls showing the respective localization of χ_{15F11}^{HA} and χ_{2E2}^{FLAG} in living cells lacking the HIV protease epitope-tagged histone H2B (chimeric anti-HIV scFv, green). Scale bars: 10 μm.

FIG. 22A is an illustration showing how to screen the original and the truncated binding epitope tags of the anti-HIV protease frankenbodies in living U2OS cells.

FIG. 22B shows the sequences of the original and truncated binding epitope tags (SEQ ID NO: 21 and 22, respectively).

FIG. 22C has images of representative cells showing the respective localization of the anti-HIV protease frankenbody in living U2OS cells co-expressing either the original epitope or the truncated epitope-tagged histone H2B χ_{15F11}^{HIV}, green; H2B-mCh-OrigTag or H2B-mCh-TrunTag, magenta). Scale bars: 10 μm.

FIG. 23A is an illustration showing how to design a chimeric anti-cat allergen scFv using wildtype scFv (wtCat-scFv) CDRs and stable scFv scaffolds.

FIG. 23B shows a sequence identity analysis of the wtCat-scFv with five intracellular scFv scaffolds.

FIG. 23C depicts the published binding epitope tag sequence of the wtCat-scFv (SEQ ID NO: 48).

FIG. 23D is a cartoon showing how to screen the chimeric anti-cat allergen scFv in living U2OS cells. The sequence of the cat allergen tag used in screening is shown in FIG. 23C. The number of epitope tags in the target protein can vary.

FIG. 23E is a representative cell image showing the respective localization of a good chimeric anti-cat allergen scFv in living U2OS cells co-expressing cat allergen epitope-tagged histone H2B (chimeric anti-cat allergen scFv, green; 4×CatTag-mCh-H2B, magenta). Scale bars: 10 μm.

### DETAILED DESCRIPTION

The present disclosure provides methods to engineer single chain variable fragments (scFv's) that specifically bind a plurality of antigens in vivo, as well as novel scFv's produced therefrom. Generally speaking, scFv's of the present disclosure have a common scaffold but different hypervariable regions. Advantageously, scFv's described herein have many live cell imaging applications, such as visualizing and quantifying the co-translational dynamics of nascent peptide chains, capturing the dynamics of short-lived proteins, tracking single molecules for extended periods of time, and selectively tracking the spatiotemporal dynamics of post-translational modifications and protein conformational change. scFv's described herein also may be genetically encoded, which offers further advantages, including but not limited to transient or stable expression in all cell types. Other aspects of scFv's of this disclosure and their uses are described more thoroughly below.

Several definitions that apply throughout this disclosure will now be presented. Unless defined otherwise, all technical and scientific terms used herein have the same meaning as commonly understood by one of ordinary skill in the art to which embodiments of the invention pertain.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result. In some instances, the term “about” may include numerical values that are rounded to the nearest significant figure.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes.

The term “antibody,” as used herein, is used in the broadest sense and encompasses various antibody and antibody-like structures, including but not limited to full-length monoclonal, polyclonal, and multispecific (e.g., bispecific, trispecific, etc.) antibodies, as well as heavy chain antibodies and antibody fragments provided they exhibit the desired antigen-binding activity. The domain(s) of an antibody involved in binding an antigen is referred to as a “variable region” or “variable domain,” and is described in further detail below. A single variable domain may be sufficient to confer antigen-binding specificity. An “isolated” antibody is one which has been separated from a component of its natural environment. For instance, an isolated antibody may be purified to greater than 95% or 99% purity as determined by methods known in the art.

The terms “full length antibody” and “intact antibody” may be used interchangeably, and refer to an antibody having a structure substantially similar to a native antibody structure or having heavy chains that contain an Fc region as defined herein. The basic structural unit of a native antibody comprises a tetramer. Each tetramer is composed of two identical pairs of polypeptide chains, each pair having one “light” chain (about 25 kDa) and one “heavy” chain (about 50-70 kDa). Light chains are classified as gamma, mu, alpha, and lambda. Heavy chains are classified as gamma, mu, alpha, delta, or epsilon, and define the antibody's isotype as IgG, IgM, IgA, IgD and IgE, respectively. The amino-terminal portion of each light and heavy chain includes a variable region of about 100 to about 120 or more amino acids primarily responsible for antigen recognition (VL and VH, respectively). The carboxy-terminal portion of each chain defines a constant region primarily responsible for effector function. Within light and heavy chains, the variable and constant regions are joined by a “J” region of about 12 or more amino acid sequences, with the heavy chain also including a “D” region of about 10 more amino acid sequences. Intact antibodies are properly cross-linked via disulfide bonds, as is known in the art.

The variable regions (also referred to as “variable domains”) of the heavy chain and the light chain of an antibody generally have similar structures, with each variable region comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL may be sufficient to confer antigen-binding specificity.

“Framework region” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence: FR1-HVR1-FR2-HVR2-FR3-HVR3-FR4. The FR domains of a heavy chain and a light chain may differ, as is known in the art.

The term “hypervariable region” or “HVR” as used herein refers to each of the regions of a variable domain which are hypervariable in sequence (also commonly referred to as “complementarity determining regions” or “CDR”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). As used herein, “an HVR derived from a variable region” refers to an HVR that has no more than two amino acid substitutions, as compared to the corresponding HVR from the original variable region. Exemplary HVRs herein include: (a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)); (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); (c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and (d) combinations of (a), (b), and/or (c), as defined below for various antibodies of this disclosure. Unless otherwise indicated, HVR residues and other residues in the variable domain (e.g., FR residues) are numbered herein according to Kabat et al., supra.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “variant Fc region” comprises an amino acid sequence that can differ from that of a native Fc region by virtue of one or more amino acid substitution(s) and/or by virtue of a modified glycosylation pattern, as compared to a native Fc region or to the Fc region of a parent polypeptide. In an example, a variant Fc region can have from about one to about ten amino acid substitutions, or from about one to about five amino acid substitutions in a native sequence Fc region or in the Fc region of the parent polypeptide. The variant Fc region herein may possess at least about 80% homology, at least about 90% homology, or at least about 95% homology with a native sequence Fc region and/or with an Fc region of a parent polypeptide.

An “antibody fragment” refers to a molecule other than an intact antibody that comprises a portion of an intact antibody that binds the antigen to which the intact antibody binds. Non-limiting examples of antibody fragments include but are not limited to Fv, Fab, Fab′, Fab′-SH, F(ab′)₂; single-chain forms of antibodies and higher order variants thereof; single-domain antibodies, and multispecific antibodies formed from antibody fragments. Single-chain forms of antibodies, and their higher order forms, may include, but are not limited to, single-domain antibodies, single chain variant fragments (scFvs), divalent scFvs (di-scFvs), trivalent scFvs (tri-scFvs), tetravalent scFvs (tetra-scFvs), diabodies, and triabodies and tetrabodies.

ScFv's are comprised of heavy and light chain variable regions connected by a linker. In most instances, but not all, the linker may be a peptide.

A “single-domain antibody” refers to an antibody fragment consisting of a single, monomeric variable antibody domain.

Multispecific antibodies include bi-specific antibodies, tri-specific, or antibodies of four or more specificities. Multispecific antibodies may be created by combining the heavy and light chains of one antibody with the heavy and light chains of one or more other antibodies. These chains can be covalently linked.

“Monoclonal antibody” refers to an antibody that is derived from a single copy or clone, including e.g., any eukaryotic, prokaryotic, or phage clone. “Monoclonal antibody” is not limited to antibodies produced through hybridoma technology. Monoclonal antibodies can be produced using hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

A “heavy chain antibody” refers to an antibody that consists of two heavy chains. A heavy chain antibody may be an IgG-like antibody from camels, llamas, alpacas, sharks, etc., or an IgNAR from a cartiliaginous fish.

The term “specifically binds,” as used herein, means that an antibody or a protein comprising an scFV does not cross react to a significant extent with other epitopes on the protein of interest, or on other proteins in general.

The term “CDR grafting” means replacing the complementarity determining regions (CDRs) of a variable region from a first antibody with the CDRs of a variable region from a second antibody. “CDR grafting” and “HVR grafting” can be used interchangeably. The term “donor antibody,” as used herein, refers to the “first antibody”—i.e., the antibody from which the CDRs are obtained.

The term “transfection” as used herein refers to the introduction of foreign DNA into a cell. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics. The term “stable transfection” or “stably transfected” refers to the introduction and integration of foreign DNA into the genome of the transfected cell. The term “transient transfection” or “transiently transfected” refers to the introduction of foreign DNA into a cell where the foreign DNA does not integrate into the genome of the transfection cell.

The term “15F11” or “scFv 15F11” refers to an scFv that specifically binds histone H4 mono-methylated at Lysine 2 described in Sato et al., J. Mol. Biol., 2016: pp. 3885-3902, VOL. 428. The amino acid sequence of the heavy chain variable domain (VH) of 15F11 is SEQ ID NO: 9. The amino acid sequences of the VH framework regions are SEQ ID NO: 25, SEQ ID NO: 26, SEQ ID NO: 27, and SEQ ID NO: 28 (FR1, FR2, FR3, and FR4, respectively). The amino acid sequence of the light chain variable domain (VL) of 15F11 is SEQ ID NO: 10. The amino acid sequences of the VL framework regions are SEQ ID NO: 29, SEQ ID NO: 30, SEQ ID NO: 31, and SEQ ID NO: 32 (FR1, FR2, FR3, and FR4, respectively).

I. scFV

One aspect of the present disclosure encompasses a plurality of single chain variable fragments (scFv's, or each an scFv) that function (e.g., specifically bind their antigen) in reducing compartment(s) of a cell. Non-limiting examples of reducing compartments of a cell (prokaryotic/eukaryotic) include the cytoplasm, the nucleus, the mitochondria, and the periplasm. Methods and reagents for evaluating the ability of an scFv to function in a reducing compartment of a cell are detailed in the Examples. Briefly, a cytoplasmic, a membrane, a mitochondrial, or a nuclear protein comprising an epitope to which the scFv binds is expressed in a cell and the scFv, labeled with a detection agent, is either co-expressed in the cell or loaded into the cell, and the cell is imaged. A uniform (e.g., diffuse) pattern of the detection agent indicates non-specific binding, while co-localization of the scFv and the epitope-tagged protein indicates specific binding.

An scFV of the present disclosure is comprised of a heavy chain variable region (VH), a light chain variable region (VL) and a linker connecting the VH and the VL. In some embodiments, the VH is at the amino terminal side of the linker and the VL is at the carboxy terminal side of the linker. In other embodiments, the VL is at the amino terminal side of the linker and the VH is at the carboxy terminal side of the linker. The VH and the VL have the general formula FR1-HVR1-FR2-HVR2-FR3-HVR3-FR4 (Formula I), wherein FR is framework region and HVR is hypervariable region, as defined above, and—is a peptide bond in each instance.

Length and composition of the linker connecting the VH and the VL may vary provided the linker does not interfere with proper folding of the variable domains on either side or create an active binding site. Suitable linkers may also allow for stabilization and/or solubilization of the variable domains. Typically the linker is a peptide linker. A linker peptide may be from about 5 to 30 amino acids in length, or from about 10 to 25 amino acids in length. In preferred embodiments, a linker peptide is rich in glycine, as well as serine and/or threonine. Charged residues such as Glu and Lys may be interspersed to enhance the solubility. Suitable peptide linkers are known in the art. In an exemplary embodiment, a linker comprises (GGGGS)ₙ, wherein n is 1 to 10, preferably 1 to 6, or more preferably 2 to 5. In another exemplary embodiment, a linker comprises (GGGGS)ₙ, wherein n is 2, 3, 4, or 5. In another exemplary embodiment, a linker comprises (GGGGS)ₙ, wherein n is 3 or 4. Linkers comprising (GGGGS)ₙ may further comprise 1 to 3 amino acids on either, or both sides.

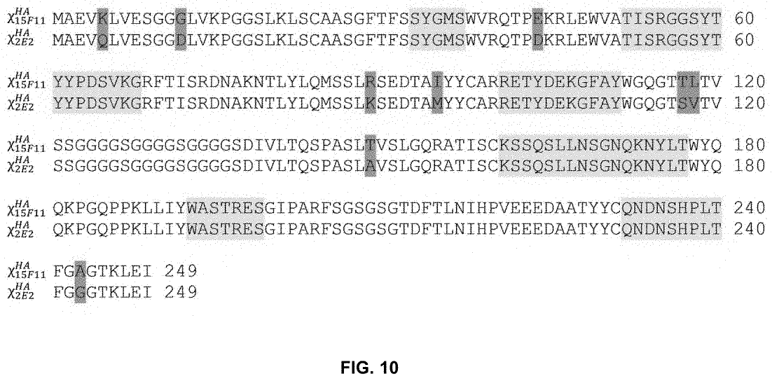

scFv's of the present disclosure have variable domains with substantially similar framework regions as the corresponding framework regions from 15F11. A “substantially similar framework region” means the framework region (e.g., FR1, FR2, FR3, or FR4) has zero to eight amino acid substitutions, as compared to the corresponding framework region from 15F11. For example, the number of amino acid substitutions may be zero, one, two, three, four, five, six, seven, or eight. Smaller ranges may also be defined, for instance, zero to four amino acid substitutions, or zero to two amino acid substitutions. A substantially similar framework region may possess at least about 75% sequence identity, at least about 78% sequence identity, or more with the corresponding framework region from 15F11. Sequence identity can be determined by sequence alignment algorithms, such as clustal, BLAST, and the like, as is routine in the art. For instance, 2E2-HA-FB (SEQ ID NO: 12) has a VH with substantially similar framework regions as the corresponding framework regions from the VH of 15F11. Specifically, the number of amino acid substitutions in FR1, FR2, FR3, and FR4 of 2E2-HA-FB's VH compared to the corresponding framework regions in 15F11 is 2, 1, 2, 2, respectively; and FR1, FR2, FR3, and FR4 from the two scFv's have about 94%, about 93%, about 94%, and about 78% identity, respectively. 2E2-HA-FB also has a VL with substantially similar framework regions as the corresponding framework regions from the VL of 15F11. See FIG. 10. Positions other than those exemplified by 2E2-HA-FB may also be mutated in the framework regions provided the resulting scFV also functions in reducing compartment(s) of a cell.

In some embodiments, an scFv of the present disclosure comprises a heavy chain variable domain wherein the amino acid sequence of FR1, FR2, FR3 and FR4 (collectively) has at least 80%, at least 81%, at least 82%, or at least 83% identity to the amino acid sequence of the VH framework regions of 15F11, and a light chain variable domain wherein the amino acid sequence of FR1, FR2, FR3 and FR4 (collectively) has at least 65%, at least 66%, at least 67%, or at least 68% identity to the amino acid sequence of the VL framework regions of 15F11.

For instance, an scFv of the present disclosure may comprise a heavy chain variable domain wherein the amino acid sequence of FR1, FR2, FR3 and FR4 (collectively) has about 83% identity or more to the amino acid sequence of the VH framework regions of 15F11, and a light chain variable domain wherein the amino acid sequence of FR1, FR2, FR3 and FR4 (collectively) has about 69% identity or more to the amino acid sequence of the VH framework regions of 15F11.

In another example, an scFv of the present disclosure may comprise a heavy chain variable domain wherein the amino acid sequence of FR1, FR2, FR3 and FR4 (collectively) has about 84% identity or more to the amino acid sequence of the VH framework regions of 15F11, and a light chain variable domain wherein the amino acid sequence of FR1, FR2, FR3 and FR4 (collectively) has about 70% identity or more to the amino acid sequence of the VH framework regions of 15F11.

Sequence identity across the collective framework regions can be determined by sequence alignment algorithms, such as clustal, BLAST, and the like, as is routine in the art. To make this determination, the amino acid sequences of the HVR regions are first removed from the amino acid sequences of the variable domains, resulting in an amino sequence consisting of the framework regions only, and then the alignment is performed.

In further embodiments, an scFv of the present disclosure may also comprise a minimum sequence identity across the entire variable domain, in addition to the requirement for a substantially similar framework region. More specifically, scFv's of the present disclosure may have variable domains with amino acid sequences that have at least 70% identity to SEQ ID NO: 9 (for the VH) and at least 60% identity to SEQ ID NO: 10 (for the VL). In one example, the amino acid sequence of the VH may have at least 70%, at least 71%, at least 72%, at least 73%, or at least 74% identity to SEQ ID NO: 9, and the VL may have at least 60% identity to SEQ ID NO: 10. In another example, the amino acid sequence of the VH may have at least 70%, at least 71%, at least 72%, at least 73%, or at least 74% identity to SEQ ID NO: 9, and the VL may have at least 61% identity to SEQ ID NO: 10. In another example, the amino acid sequence of the VH may have at least 70%, at least 71%, at least 72%, at least 73%, or at least 74% identity to SEQ ID NO: 9, and the VL may have at least 62% identity to SEQ ID NO: 10. In another example, the amino acid sequence of the VH may have at least 70%, at least 71%, at least 72%, at least 73%, or at least 74% identity to SEQ ID NO: 9, and the VL may have at least 63% identity to SEQ ID NO: 10. In another example, the amino acid sequence of the VH may have about 70%, to about 99% identity to SEQ ID NO: 9, and the VL may have at least 60%, at least 61%, at least 62%, or at least 63% identity to SEQ ID NO: 10. In another example, the amino acid sequence of the VH may have at least 70%, at least 71%, at least 72%, at least 73%, or at least 74% identity to SEQ ID NO: 9, and the VL may have about 60% to about 99% identity to SEQ ID NO: 10. In another example, the amino acid sequence of the VH may have at about 70% to about 99% identity to SEQ ID NO: 9, and the VL may have about 60% to about 99% identity to SEQ ID NO: 10.

In one example, an scFV of the present disclosure may comprise a heavy chain variable region (VH) of formula (I) with an amino acid sequence that has about 70% but less than 100% identity to SEQ ID NO: 9, and a light chain variable region VL of formula (I) with an amino acid sequence that has about 60% but less than 100% to SEQ ID NO: 10, wherein each framework region (FR) of the VH and the VL has at least 75% identity to the corresponding FR of SEQ ID NO: 9 and SEQ ID NO: 10, respectively.

In another example, an scFV of the present disclosure may comprise a heavy chain variable region (VH) of formula (I) with an amino acid sequence that has about 70% but less than 100% identity to SEQ ID NO: 9 and wherein the amino acid sequence of FR1, FR2, FR3 and FR4 (collectively) has at least 80%, at least 81%, at least 82%, or at least 83% identity to the amino acid sequence of the VH framework regions of 15F11; and a light chain variable region VL of formula (I) with an amino acid sequence that has about 60% but less than 100% to SEQ ID NO: 10, wherein the amino acid sequence of FR1, FR2, FR3 and FR4 (collectively) has at least 65%, at least 66%, at least 67%, or at least 68% identity to the amino acid sequence of the VL framework regions of 15F11.

In another example, an scFV of the present disclosure may comprise a heavy chain variable region (VH) of formula (I) with an amino acid sequence that has about 70% but less than 100% identity to SEQ ID NO: 9 and wherein the amino acid sequence of FR1, FR2, FR3 and FR4 (collectively) has about 83% identity or more to the amino acid sequence of the VH framework regions of 15F11; and a light chain variable region VL of formula (I) with an amino acid sequence that has about 60% but less than 100% to SEQ ID NO: 10, wherein the amino acid sequence of FR1, FR2, FR3 and FR4 (collectively) has about 69% identity or more to the amino acid sequence of the VH framework regions of 15F11.

In another example, an scFV of the present disclosure may comprise a heavy chain variable region (VH) of formula (I) with an amino acid sequence that has about 70% but less than 100% identity to SEQ ID NO: 9 and wherein the amino acid sequence of FR1, FR2, FR3 and FR4 (collectively) has about 84% identity or more to the amino acid sequence of the VH framework regions of 15F11; and a light chain variable region VL of formula (I) with an amino acid sequence that has about 60% but less than 100% to SEQ ID NO: 10, wherein the amino acid sequence of FR1, FR2, FR3 and FR4 (collectively) has about 70% identity or more to the amino acid sequence of the VH framework regions of 15F11.

In another example, an scFV of the present disclosure may comprise a heavy chain variable region (VH) of formula (I) with an amino acid sequence that has about 70% but less than 100% identity to SEQ ID NO: 9, and a light chain variable region VL of formula (I) with an amino acid sequence that has about 60% but less than 100% identity to SEQ ID NO: 10, wherein (a) FR1 of the VH has about 90% identity or greater to SEQ ID NO: 25, FR2 of the VH has about 90% identity or greater to SEQ ID NO: 26, FR3 of the VH has about 90% identity or greater to SEQ ID NO: 27, and FR4 of the VH has about 75% identity or greater to SEQ ID NO: 28; and (b) FR1 of the VL has about 90% identity or greater to SEQ ID NO: 29, FR2 of the VL has about 90% identity or greater to SEQ ID NO: 30, FR3 of the VL has about 90% identity or greater SEQ ID NO: 31, and FR4 of the VL has about 75% identity or greater to SEQ ID NO: 32.

In another example, an scFV of the present disclosure may comprise a heavy chain variable region (VH) of formula (I) with an amino acid sequence that has about 70% but less than 100% identity to SEQ ID NO: 9, and a light chain variable region VL of formula (I) with an amino acid sequence that has about 60% but less than 100% identity to SEQ ID NO: 10, wherein (a) FR1 of the VH has about 93% identity or greater to SEQ ID NO: 25, FR2 of the VH has about 93% identity or greater to SEQ ID NO: 26, FR3 of the VH has about 93% identity or greater to SEQ ID NO: 27, and FR4 of the VH has about 78% identity or greater to SEQ ID NO: 28; and (b) FR1 of the VL has about 93% identity or greater to SEQ ID NO: 29, FR2 of the VL has about 93% identity or greater to SEQ ID NO: 30, FR3 of the VL has about 93% identity or greater to SEQ ID NO: 31, and FR4 of the VL has about 78% identity or greater to SEQ ID NO: 32.

In another example, an scFV of the present disclosure may comprise a heavy chain variable region (VH) of formula (I) with an amino acid sequence that has about 70% but less than 100% identity to SEQ ID NO: 9, and a light chain variable region VL of formula (I) with an amino acid sequence that has about 60% but less than 100% identity to SEQ ID NO: 10, wherein each framework region (FR) of the VH and the VL has no more than two amino acid substitutions as compared to the corresponding to FR of SEQ ID NO: 9 and SEQ ID NO: 10, respectively.

In another example, an scFV of the present disclosure may comprise a heavy chain variable region (VH) of formula (I) with an amino acid sequence that has about 70% but less than 100% identity to SEQ ID NO: 9, and a light chain variable region VL of formula (I) with an amino acid sequence that has about 60% but less than 100% identity to SEQ ID NO: 10, wherein (a) FR1 of the VH has no more than two amino acid substitutions as compared to SEQ ID NO: 25, FR2 of the VH has no more than one amino acid substitution as compared to SEQ ID NO: 26, FR3 of the VH has no more than two amino acid substitutions as compared to SEQ ID NO: 27, and FR4 of the VH has no more than two amino acid substitutions as compared to SEQ ID NO: 28; and (b) FR1 of the VL has no more than two amino acid substitutions as compared to SEQ ID NO: 29, FR2 of the VL has no more than one amino acid substitution as compared to SEQ ID NO: 30, FR3 of the VL has no more than two amino acid substitutions as compared to SEQ ID NO: 31, and FR4 of the VL no more than two amino acid substitutions as compared to SEQ ID NO: 32.

In another example, an scFV of the present disclosure may comprise a heavy chain variable region (VH) of formula (I) with an amino acid sequence that has about 70% identity but less than 100% identity to SEQ ID NO: 9, and a light chain variable region VL of formula (I) with an amino acid sequence that has about 60% but less than 100% identity to SEQ ID NO: 10, wherein (a) FR1 of the VH has no more than two amino acid substitutions as compared to SEQ ID NO: 25, FR2 of the VH has no more than one amino acid substitution as compared to SEQ ID NO: 26, FR3 of the VH has no more than two amino acid substitutions as compared to SEQ ID NO: 27, and FR4 of the VH has no more than two amino acid substitutions as compared to SEQ ID NO: 28; and (b) FR1 of the VL has no more than one amino acid substitution as compared to SEQ ID NO: 29, FR2 of the VL has no more than one amino acid substitution as compared to SEQ ID NO: 30, FR3 of the VL has no more than one amino acid substitution as compared to SEQ ID NO: 31, and FR4 of the VL no more than one amino acid substitution as compared to SEQ ID NO: 32.

In another example, an scFV of the present disclosure may comprise a heavy chain variable region (VH) of formula (I) with an amino acid sequence that has about 70% identity but less than 100% identity to SEQ ID NO: 9, and a light chain variable region VL of formula (I) with an amino acid sequence that has about 60% but less than 100% identity to SEQ ID NO: 10, wherein (a) FR1 of the VH has no more than two amino acid substitutions as compared to SEQ ID NO: 25, FR2 of the VH has no more than one amino acid substitution as compared to SEQ ID NO: 26, FR3 of the VH has no more than two amino acid substitutions as compared to SEQ ID NO: 27, and FR4 of the VH has no more than two amino acid substitutions as compared to SEQ ID NO: 28; and (b) FR1 of the VL has no more than one amino acid substitution as compared to SEQ ID NO: 29, FR2 of the VL is SEQ ID NO: 30, FR3 of the VL is SEQ ID NO: 31, and FR4 of the VL no more than one amino acid substitution as compared to SEQ ID NO: 32.

In another example, an scFV of the present disclosure may comprise a heavy chain variable region (VH) of formula (I) with an amino acid sequence that has about 70% but less than 100% identity to SEQ ID NO: 9, and a light chain variable region VL of formula (I) with an amino acid sequence that has about 60% but less than 100% identity to SEQ ID NO: 10, wherein (a) FR1 of the VH is SEQ ID NO: 1, FR2 of the VH is SEQ ID NO: 2, FR3 of the VH is SEQ ID NO: 3, and FR4 of the VH is SEQ ID NO: 4; and (b) FR1 of the VL is SEQ ID NO: 5, FR2 of the VL is SEQ ID NO: 6, FR3 of the VL is SEQ ID NO: 7, and FR4 of the VL is SEQ ID NO: 8.

scFv's of the present disclosure may be described as derivatives of 15F11 given the sequence similarity described above, but all scFv's described herein have a different antigen-binding specificity than 15F11. In certain embodiments, an scFV may have a different specificity than 15F11 and 2E2. Accordingly, all scFv's of the present disclosure have at least one HVR with an amino acid sequence that differs from the corresponding hypervariable region of SEQ ID NO: 9 or SEQ ID NO: 10.

There are multiple approaches by which a skilled artisan can identify suitable amino acid sequences for the HVRs. For instance, as detailed in Example 2, a skilled artisan may begin with a plurality of known antibody sequences, identify one or more antibody with substantially similar framework regions as 15F11 (each a donor antibody), and then graft the CDRs from the donor antibody onto a scaffold that has substantially similar framework regions as the corresponding framework regions from 15F11. Alternatively, a skilled artisan may generate a plurality of scFvs and then screen the plurality of scFv's against an antigen to select for scFv's that have a desired specificity. The scFvs that result from the screen may be further characterized in terms of the % identity of each scFv's VH and VL to SEQ ID NO: 9 and SEQ ID NO: 10.

In each of the above embodiments, an scFv of the present disclosure may further comprise HVRs derived from the antibody F11.2.32 (see, e.g., Protein Database Bank ID 2HRP). Alternatively, in each of the above embodiments, an scFv of the present disclosure may further comprise HVRs derived from the antibody REGN1909 (see, e.g., Protein Database Bank ID 5VYF). Alternatively, in each of the above embodiments, an scFv of the present disclosure may further comprise HVRs derived from the antibody 12CA5 (Protein Database Bank ID 2HRP). Alternatively, in each of the above embodiments, an scFv of the present disclosure may further comprise HVRs derived from SEQ ID NOs: 42, 43, 44, 45, 46 and 47. Alternatively, in each of the above embodiments, an scFv of the present disclosure may further comprise HVRs derived from an antibody listed in Table A, identified by the Protein Database Bank (PDB) ID. Amino acid sequences for the antibodies identified by a PDB ID can be obtained from Protein Database Bank, and the HVRs identified as described herein.

TABLE A

| Rank | PDB |
|---|---|
| 1 | 5B3N |
| 2 | 15F11 |
| 3 | 12C8 |
| 4 | 2E2 |
| 5 | 22G3 |
| 6 | 6D6A5 |
| 7 | 5DQD |
| 8 | 5DQJ |
| 9 | 2OR9 |
| 10 | 2ORB |
| 11 | 4ODS |
| 12 | 5DQ9 |
| 13 | 2AAB |
| 14 | 1MF2 |
| 15 | 2HRP |
| 16 | 1EJO |
| 17 | 5XCS |
| 18 | 1HIN |
| 19 | 1IFH |
| 20 | 3V6F |
| 21 | 4AEI |
| 22 | 22A9 |
| 23 | 3LS4 |
| 24 | 3LS5 |
| 25 | 4DTG |
| 26 | 2A1W |
| 27 | 2AI0 |
| 28 | 2A77 |
| 29 | 5DO2 |
| 30 | 5I1I |
| 31 | 16D4 |
| 32 | 5CSZ |
| 33 | 1H3P |
| 34 | 5I1D |
| 35 | 1KFA |
| 36 | 1IGF |
| 37 | 2IGF |
| 38 | 5XS7 |
| 39 | 3I2C |
| 40 | 2H1P |
| 41 | 5DR5 |
| 42 | 1SEQ |
| 43 | 5DLM |
| 44 | 3INU |
| 45 | 3ZTN |
| 46 | 4OQT |
| 47 | 5I1G |
| 48 | 5NBI |
| 49 | 3JBA |
| 50 | 5I1A |
| 51 | 4ZPT |
| 52 | 4ZPV |
| 53 | 1CLZ |
| 54 | 4HZL |
| 55 | 4LQF |
| 56 | 5TZU |
| 57 | 1QKZ |
| 58 | 5V2A |
| 59 | 4LVH |
| 60 | 3HR5 |
| 61 | 4OUU |
| 62 | 4QXU |
| 63 | 5XJ4 |
| 64 | 6BF4 |
| 65 | 5NB5 |
| 66 | 6AMJ |
| 67 | 6AMM |
| 68 | 4S2S |
| 69 | 1CLY |
| 70 | 1UCB |
| 71 | 3GIZ |
| 72 | 2DQT |
| 73 | 2DQU |
| 74 | 2DTM |
| 75 | 4LU5 |
| 76 | 2MCP |
| 77 | 4JFX |
| 78 | 4JFY |
| 79 | 4JFZ |
| 80 | 4JG0 |
| 81 | 4JG1 |
| 82 | 5EA0 |
| 83 | 5BK3 |
| 84 | 5I1H |
| 85 | 5I1E |
| 86 | 3VRL |
| 87 | 6AO0 |
| 88 | 6ATT |
| 89 | 5GGU |
| 90 | 5GGV |
| 91 | 1LO0 |
| 92 | 1LO2 |
| 93 | 1LO3 |
| 94 | 5VEB |
| 95 | 5I19 |
| 96 | 6GHG |
| 97 | 5I1C |
| 98 | 4M48 |
| 99 | 4XP4 |
| 100 | 4XPA |
| 101 | 4XPF |
| 102 | 4XPG |
| 103 | 4XP1 |
| 104 | 4XP5 |
| 105 | 4XP6 |
| 106 | 4XPT |
| 107 | 4XP9 |
| 108 | 4XPH |
| 109 | 4P3C |
| 110 | 4P3D |
| 111 | 1MQK |
| 112 | 1QLE |
| 113 | 5VYF |
| 114 | 5U4R |
| 115 | 4P59 |
| 116 | 5WI9 |
| 117 | 1FRG |
| 118 | 5DWU |
| 119 | 4XNU |
| 120 | 4XNX |
| 121 | 4XPB |
| 122 | 3RA7 |
| 123 | 7D4 |
| 124 | 4FQL |
| 125 | 4Z0B |
| 126 | 6B0A |
| 127 | 2J4W |
| 128 | 4ODV |
| 129 | 4ODW |
| 130 | 3CFB |
| 131 | 3CFC |
| 132 | 6BQB |
| 133 | 3KYK |
| 134 | 4CNI |
| 135 | 5TRU |
| 136 | 4NYL |
| 137 | 6ANP |
| 138 | 4X7S |
| 139 | 4X7T |
| 140 | 4U6V |
| 141 | 3KR3 |
| 142 | 4M6O |
| 143 | 1FGV |
| 144 | 5TL5 |
| 145 | 1TZH |
| 146 | 1ZA3 |
| 147 | 6CNR |
| 148 | 6CO3 |
| 149 | 5G64 |
| 150 | 4KVC |
| 151 | 5GGQ |
| 152 | 5GGR |
| 153 | 3WD5 |
| 154 | FLAG |
| 155 | 2UZI |

In an exemplary embodiment, an scFV of the present disclosure comprises a heavy chain variable region (VH), a light chain variable region (VL) and a linker connecting the VH and the VL, wherein the VH has an amino acid sequence corresponding to amino acids 1 to 120 of SEQ ID NO: 11 and the VL has an amino acid sequence corresponding to amino acids 138 to 249 of SEQ ID NO: 11. In some embodiments, the linker is a peptide linker. In further embodiments, the linker is a peptide linker consisting of about 5 to about 30 amino acids, or about 10 to about 25 amino acids. In still further embodiments, the peptide linker of about 5 to about 30 amino acids comprises (GGGGS)ₙ, wherein n is 1 to 10, preferably 1 to 6, or more preferably 2 to 5. Linkers comprising (GGGGS)ₙ may further comprise 1 to 3 amino acids on either, or both sides.

In an exemplary embodiment, an scFV of the present disclosure comprises a heavy chain variable region (VH), a light chain variable region (VL) and a linker connecting the VH and the VL, wherein the VH has an amino acid sequence corresponding to amino acids 1 to 120 of SEQ ID NO: 12 and the VL has an amino acid sequence corresponding to amino acids 138 to 249 of SEQ ID NO: 12. In some embodiments, the linker is a peptide linker. In further embodiments, the linker is a peptide linker consisting of about 5 to about 30 amino acids, or about 10 to about 25 amino acids. In still further embodiments, the peptide linker of about 5 to about 30 amino acids comprises (GGGGS)ₙ, wherein n is 1 to 10, preferably 1 to 6, or more preferably 2 to 5. Linkers comprising (GGGGS)ₙ may further comprise 1 to 3 amino acids on either, or both sides.

In an exemplary embodiment, an scFV of the present disclosure comprises a heavy chain variable region (VH), a light chain variable region (VL) and a linker connecting the VH and the VL, wherein the VH has an amino acid sequence corresponding to amino acids 1 to 118 of SEQ ID NO: 13 and the VL has an amino acid sequence corresponding to amino acids 136 to 246 of SEQ ID NO: 13. In some embodiments, the linker is a peptide linker. In further embodiments, the linker is a peptide linker consisting of about 5 to about 30 amino acids, or about 10 to about 25 amino acids. In still further embodiments, the peptide linker of about 5 to about 30 amino acids comprises (GGGGS)ₙ, wherein n is 1 to 10, preferably 1 to 6, or more preferably 2 to 5. Linkers comprising (GGGGS)ₙ may further comprise 1 to 3 amino acids on either, or both sides.

In an exemplary embodiment, an scFV of the present disclosure comprises a heavy chain variable region (VH), a light chain variable region (VL) and a linker connecting the VH and the VL, wherein the VH has an amino acid sequence corresponding to amino acids 1 to 118 of SEQ ID NO: 14 and the VL has an amino acid sequence corresponding to amino acids 136 to 246 of SEQ ID NO: 14. In some embodiments, the linker is a peptide linker. In further embodiments, the linker is a peptide linker consisting of about 5 to about 30 amino acids, or about 10 to about 25 amino acids. In still further embodiments, the peptide linker of about 5 to about 30 amino acids comprises (GGGGS)ₙ, wherein n is 1 to 10, preferably 1 to 6, or more preferably 2 to 5. Linkers comprising (GGGGS)ₙ may further comprise 1 to 3 amino acids on either, or both sides.

In an exemplary embodiment, an scFV of the present disclosure comprises a heavy chain variable region (VH), a light chain variable region (VL) and a linker connecting the VH and the VL, wherein the VH has an amino acid sequence corresponding to amino acids 1 to 125 of SEQ ID NO: 15 and the VL has an amino acid sequence corresponding to amino acids 143 to 252 of SEQ ID NO: 15. In some embodiments, the linker is a peptide linker. In further embodiments, the linker is a peptide linker consisting of about 5 to about 30 amino acids, or about 10 to about 25 amino acids. In still further embodiments, the peptide linker of about 5 to about 30 amino acids comprises (GGGGS)ₙ, wherein n is 1 to 10, preferably 1 to 6, or more preferably 2 to 5. Linkers comprising (GGGGS)ₙ may further comprise 1 to 3 amino acids on either, or both sides.

In an exemplary embodiment, an scFV of the present disclosure comprises a heavy chain variable region (VH), a light chain variable region (VL) and a linker connecting the VH and the VL, wherein the VH has an amino acid sequence corresponding to amino acids 1 to 125 of SEQ ID NO: 16 and the VL has an amino acid sequence corresponding to amino acids 143 to 252 of SEQ ID NO: 16. In some embodiments, the linker is a peptide linker. In further embodiments, the linker is a peptide linker consisting of about 5 to about 30 amino acids, or about 10 to about 25 amino acids. In still further embodiments, the peptide linker of about 5 to about 30 amino acids comprises (GGGGS)ₙ, wherein n is 1 to 10, preferably 1 to 6, or more preferably 2 to 5. Linkers comprising (GGGGS)ₙ may further comprise 1 to 3 amino acids on either, or both sides.

In an exemplary embodiment, an scFV of the present disclosure comprises a heavy chain variable region (VH), a light chain variable region (VL) and a linker connecting the VH and the VL, wherein the VH has an amino acid sequence corresponding to amino acids 1 to 115 of SEQ ID NO: 17 and the VL has an amino acid sequence corresponding to amino acids 133 to 238 of SEQ ID NO: 17. In some embodiments, the linker is a peptide linker. In further embodiments, the linker is a peptide linker consisting of about 5 to about 30 amino acids, or about 10 to about 25 amino acids. In still further embodiments, the peptide linker of about 5 to about 30 amino acids comprises (GGGGS)ₙ, wherein n is 1 to 10, preferably 1 to 6, or more preferably 2 to 5. Linkers comprising (GGGGS)ₙ may further comprise 1 to 3 amino acids on either, or both sides.

In an exemplary embodiment, an scFV of the present disclosure comprises a heavy chain variable region (VH), a light chain variable region (VL) and a linker connecting the VH and the VL, wherein the VH has an amino acid sequence corresponding to amino acids 1 to 115 of SEQ ID NO: 33 and the VL has an amino acid sequence corresponding to amino acids 133 to 238 of SEQ ID NO: 33. In some embodiments, the linker is a peptide linker. In further embodiments, the linker is a peptide linker consisting of about 5 to about 30 amino acids, or about 10 to about 25 amino acids. In still further embodiments, the peptide linker of about 5 to about 30 amino acids comprises (GGGGS)ₙ, wherein n is 1 to 10, preferably 1 to 6, or more preferably 2 to 5. Linkers comprising (GGGGS)ₙ may further comprise 1 to 3 amino acids on either, or both sides.

In an exemplary embodiment, an scFV of the present disclosure has an amino acid sequence corresponding to SEQ ID NO: 11 or SEQ ID NO: 12.

In an exemplary embodiment, an scFV of the present disclosure has an amino acid sequence corresponding to SEQ ID NO: 13 or SEQ ID NO: 14.

In an exemplary embodiment, an scFV of the present disclosure has an amino acid sequence corresponding to SEQ ID NO: 15 or SEQ ID NO: 16.

In an exemplary embodiment, an scFV of the present disclosure has an amino acid sequence corresponding to SEQ ID NO: 17 or SEQ ID NO: 33.

In each of the above embodiments, the scFv may be an isolated scFv. An “isolated” scFv is one which has been separated from a component of its natural environment. In some embodiments, an isolated scFv is purified to greater than 95% or 99% purity as determined by methods known in the art. Methods for purifying an scFv are described in Example 1. Additional methods are also known in the art.

scFvs of the present disclosure can be used in in vitro applications including but not limited to phage display, flow cytometry, immunohistochemistry, Western blotting, immunofluorescence applications. scFvs of the present disclosure can also be used as targeting domains for in vitro or in vivo applications. For instance, an scFv of the present disclosure can be conjugated to a payload (e.g., an enzyme or other protein, a small molecule, a toxin, etc.) and used to deliver the payload to scFv's target. scFvs of the present disclosure can also be used for live cell imaging. scFvs of the present disclosure may also be conjugated to a human constant domain (e.g. a heavy constant domain is derived from an IgG domain, such as IgG1, IgG2, IgG3, or IgG4, or a heavy chain constant domain derived from IgA, IgM, or IgE).

II. Method for Engineering an scFv for Live Cell Imaging

Another aspect of the present disclosure encompasses a method for engineering an scFv for that binds its target epitope in a reducing compartment of a cell, and therefore can be used for live cell imaging.

In one embodiment, the method comprises grafting hypervariable regions (HVRs) from a heavy chain variable region (VH) and a light chain variable region (VL) of a donor antibody onto a VH and a VL, respectively, of an scFv disclosed in Section I, wherein the donor antibody comprises a heavy chain variable domain wherein the amino acid sequence of FR1, FR2, FR3 and FR4 (collectively) has at least 80%, at least 81%, at least 82%, or at least 83% identity to the amino acid sequence of the VH framework regions of the scFv, and a light chain variable domain wherein the amino acid sequence of FR1, FR2, FR3 and FR4 (collectively) has at least 65%, at least 66%, at least 67%, or at least 68% identity to the amino acid sequence of the VL framework regions of the scFv. In further embodiments, the donor antibody may also have a VH with an amino acid sequence that has at least 70% identity to the VH of the scFv and a VL with an amino acid sequence that has at least 60% identity to the VH of the scFv. In one example, the amino acid sequence of the donor antibody's VH may have at least 70%, at least 71%, at least 72%, at least 73%, or at least 74% identity to the amino acid sequence of the scFv's VH, and the amino acid sequence of the donor antibody's VL may have at least 60%, at least 61%, at least 62%, or at least 63% identity to SEQ ID NO: 10. Typically, the donor antibody is not capable of specifically binding its target epitope in a living cell. In some embodiments, the donor antibody is a monoclonal antibody. In other embodiments, the donor antibody is an scFv. In other embodiments, the donor antibody is a heavy chain antibody. Suitable scFvs are described in detail in Section I.

Methods for identifying the variable domains of an antibody, and the HVRs within a variable domain, are known in the art. See, for instance, Chothia and Lesk, J. Mol. Biol. 196:901-917 (1987), (Kabat et al., Sequences of Proteins of Immunological Interest, 5th Ed. Public Health Service, National Institutes of Health, Bethesda, Md. (1991), MacCallum et al. J. Mol. Biol. 262: 732-745 (1996). In an exemplary embodiment, HVRs are numbered according to Kabat et al., supra. HVR grafting may be achieved as detailed in the Examples, or by using recombinant DNA techniques well known in the art.

In another embodiment, the method comprises generating a plurality of nucleic acid sequences, each nucleic acid sequence encoding an scFv, screening the plurality of nucleic acid sequences (e.g., by phage display or other method known in the art) to identify an scFv that specifically binds a protein interest or a target epitope of interest, and then selecting from the scFv's that specifically bind a protein interest or a target epitope of interest an scFv with substantially similar framework regions as the corresponding framework regions from 15F11. In some embodiments, those scFvs with substantially similar framework regions as the corresponding framework regions from 15F11 may be further screened for an scFv with a VH that has an amino acid sequence with at least 70% identity to SEQ ID NO: 9 and VL that has an amino acid sequence with at least 60% identity to SEQ ID NO: 10. Preferred framework regions include those of the scFv embodiments disclosed in Section I, which are incorporated by reference into this Section.

III. Protein Comprising an scFV

Another aspect of the present disclosure encompasses a protein comprising an scFv of Section I. In some embodiments, the protein is fusion protein and further comprises one or more additional polypeptide, each polypeptide optionally connected by a peptide linker. Non-limiting examples of additional polypeptides include a tag, a sub-cellular localization signal, a cell penetrating domain, and a protein of interest. In some embodiments, the protein is protein conjugate and further comprises a non-polypeptide payload, the payload optionally connected by a flexible linker. Non-limiting examples non-polypeptide payloads include inorganic fluorescent probes, a toxin, and a chemically synthesized drug. In still further embodiments, a fusion protein or a protein conjugate may also comprise one or more cleavage site, one or more tag, a sub-cellular localization signal, a cell penetrating domain or any combination thereof. In each of the above embodiments, the protein may be an isolated protein. An isolated protein may be purified to greater than 95% purity or greater than 99% purity as determined by methods known in the art.

When the linker is present, it allows for proper folding of the scFv and prevents possible steric hindrance of the scFv and the additional domain (e.g., polypeptide, payload, or both). Suitable peptide linkers are described in Section I. Flexible non-peptide linkers are known in the art.

A payload and/or a non-peptide linker may be attached to an scFv via a reactive functional group. Polypeptides and optional peptide linkers may be attached to an scFv through chemical ligation or via reactive functional groups. Alternatively, a nucleic acid sequence encoding the additional polypeptide, and optional linker, may be fused in-frame to the nucleic acid sequence encoding the scFv such that a fusion protein is generated. In-frame means that the open reading frame (ORF) of the nucleic acid sequence encoding the scFv and the ORF encoding the additional polypeptide are maintained. In-frame insertions occur when the number of inserted nucleotides is divisible by three, which may be achieved by adding a linker of any number of nucleotides to the nucleic acid sequence encoding the additional polypeptide as applicable.

In some embodiments, the protein further comprises an inorganic fluorescent probe, optionally connected to the scFv by a linker. The inorganic fluorescent probe may be located N-terminal to or C-terminal to scFv. When the linker is present, it allows for proper folding of the scFv and prevents possible steric hindrance of the scFv and the inorganic fluorescent probe. Inorganic fluorescent probes include, but are not limited to, semiconductor nanocrystals (also called Quantum Dots, QDs), silicon nanoparticles, lanthanide-doped oxide nanoparticles, and fluorescent nanodiamonds.

In some embodiments, the protein further comprises one or more tag, optionally connected to the scFv by a linker. The tag may be located N-terminal to or C-terminal to scFv. When the linker is present, it allows for proper folding of the tag and prevents possible steric hindrance of the scFv and the tag. The linker may be the same or different than the linker of the scFv. A “tag” may be any of a number of peptide sequences known in the art to facilitate detection, purification, solubilization or the like. Non-limiting types of tags known in the art include epitope tags, affinity tags, reporters, or combinations thereof.

In some embodiments, the tag may be an epitope tag. The epitope tag may comprise a random amino acid sequence, or a known amino acid sequence. A known amino acid sequence may have, for example, antibodies generated against it. The epitope tag may be an antibody epitope tag for which commercial antibodies are available. Non-limiting examples of suitable antibody epitope tags are myc, AcV5, AU1, AU5, E, ECS, E2, FLAG, HA, Maltose binding protein, nus, Softag 1, Softag 3, Strep, SBP, Glu-Glu, HSV, KT3, S, S1, T7, V5, VSV-G, 6×His, biotin carboxyl carrier protein (BCCP), and calmodulin.

In some embodiments, the tag may be a reporter. Suitable reporters are known in the art. Non-limiting examples of reporters include affinity tags, visual reporters, or self-labeling enzyme tags. Non-limiting examples of affinity tags include hexahistidine, chitin binding protein (CBP), thioredoxin (TRX), poly(NANP), tandem affinity purification (TAP) tag, and glutathione-S-transferase (GST). Visual reporters typically result in a visual signal, such as a color change in the cell, or fluorescence or luminescence of the cell. For instance, the reporter LacZ, which encodes β-galactosidase, will turn a cell blue in the presence of a suitable substrate, such as X-gal. Other non-limiting examples of visual reporters include fluorescent proteins, bioluminescent proteins (e.g., luciferase, etc.), alkaline phosphatase, beta-galactosidase, beta-lactamase, horseradish peroxidase, and variants thereof. Also contemplated are split reporter tags. For instance, split fluorescent proteins, split luciferase, and the like. In these embodiments, an scFv of the present disclosure is labeled with one half of the split reporter and a target comprising the epitope to which the scFv specifically binds is labeled with the second half of the split reporter.

An exemplary tag is a self-labeling tag. Self-labeling tags catalyze the covalent attachment of an exogenously added synthetic ligand. Such synthetic ligands are tag specific and can be coupled to diverse useful labels, such as fluorescent dyes, affinity handles, or solid surfaces. The covalent attachment of the functionalized ligand to the enzyme tag is highly specific, happens rapidly under physiological conditions in living cells, or in chemically fixed cells, and is most importantly irreversible. Non-limiting examples of self-labeling enzyme tags include SNAP-tag, CLIP-tag, ACP-tag, MCP-tag, and HaloTag.

Another exemplary tag is a fluorescent protein. Non limiting examples of fluorescent protein visual reporters include green fluorescent proteins (e.g., GFP, GFP-2, tagGFP, turboGFP, EGFP, mEGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalamal, GFPuv, Sapphire, T-sapphire), cyan fluorescent proteins (e.g. ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein.

An scFv may be tagged with more than one tag. For instance, an scFv may be tagged with at least one, two, three, four, five, six, seven, eight, or nine tags. More than one tag may be expressed as a single polypeptide fused to an scFv. More than one tag fused to an scFv may be expressed as a single polypeptide which is cleaved into the individual tag polypeptides after translation. By way of non-limiting example, 2A peptides of picornaviruses inserted between tag polypeptides or between tag polypeptide and the scFv may result in the co-translational ‘cleavage’ of a tag and lead to expression of multiple proteins at equimolar levels.

In further embodiments, the protein may comprise a sub-cellular localization signal, such as a nuclear localization signal (NLS), a mitochondrial targeting peptide, a secretory pathway signal peptide, and the like, and optionally a linker. The sub-cellular localization signal may be located N-terminal to or C-terminal to scFv. When the linker is present, it allows for proper folding of the sub-cellular localization signal and prevents possible steric hindrance of the scFv and the sub-cellular localization signal. The linker may be the same or different than the linker of the scFv. A curated list of protein localization signals may be found in LocSigDB (genome.unmc.edu/LocSigDB). Specific nuclear localization signals are known in the art (see, e.g., Lange et al., J. Biol. Chem., 2007, 282:5101-5105). For example, in one embodiment, the NLS can be a monopartite sequence, such as PKKKRKV (SEQ ID NO: 34) or PKKKRRV (SEQ ID NO: 35). In another embodiment, the NLS can be a bipartite sequence. In still another embodiment, the NLS can be KRPAATKKAGQAKKKK (SEQ ID NO: 36).

In further embodiments, the protein may comprise at least one cell-penetrating domain and optionally a linker. The cell-penetrating domain may be located N-terminal to or C-terminal to scFv. When the linker is present, it allows for proper folding of the cell-penetrating domain and prevents possible steric hindrance of the scFv and the cell-penetrating domain. The linker may be the same or different than the linker of the scFv. In one embodiment, the cell-penetrating domain can be a cell-penetrating peptide sequence derived from the HIV-1 TAT protein. As an example, the TAT cell-penetrating sequence can be GRKKRRQRRRPPQPKKKRKV (SEQ ID NO: 37). In another embodiment, the cell-penetrating domain can be TLM (PLSSIFSRIGDPPKKKRKV; SEQ ID NO: 38), a cell-penetrating peptide sequence derived from the human hepatitis B virus. In still another embodiment, the cell-penetrating domain can be MPG (GALFLGWLGAAGSTMGAPKKKRKV; SEQ ID NO: 39 or GALFLGFLGAAGSTMGAWSQPKKKRKV; SEQ ID NO: 40). In an additional embodiment, the cell-penetrating domain can be Pep-1 (KETWWETWWTEWSQPKKKRKV; SEQ ID NO:8), VP22, a cell penetrating peptide from Herpes simplex virus, or a polyarginine peptide sequence. The cell-penetrating domain can be located at the N-terminus, the C-terminus, or in an internal location of the protein.

In further embodiments, the protein may comprise a polypeptide encoding a protein or amino sequence of interest, and a linker connecting the scFv and the polypeptide. The polypeptide may be located N-terminal to or C-terminal to scFv. The linker allows for proper folding of the polypeptide and the scFv and prevents possible steric hindrance of the scFv and the polypeptide. The linker may be the same or different than the linker of the scFv.

In each of the above embodiments, the protein may further comprise a protease cleavage site. Non-limiting examples of protease cleavage sites include a tomato etch virus (TEV) protease cleavage site, a thrombin cleavage site, a PreScisison cleavage site, or variants thereof. The amino acid sequences of these protease cleavage sites are known in art, as are additional protease cleavage sites suitable for, and commonly used in, vectors. In addition, the peptide tags SUMO and FLAG are cleaved by specific proteases without requiring the addition of an independent cleavage recognition site. In embodiments comprising at least one protease cleavage site, the protease cleavage site may be positioned between the scFv and any additional polypeptides, NLS, or cell-penetrating domains, or between the scFv and the linker when the linker is present. In this manner, the scFv may be cleaved from any fusion partner if desired.

In an exemplary embodiment, the protein comprises (a) an scFv of Section I, (b) an inorganic fluorescent probe, a fluorescent protein, a bioluminescent protein, an inorganic fluorescent probe, and/or a self-labeling tag, and (c) a peptide linker of 1 to about 30 amino acids connecting the scFv and the probe, protein, and/or tag. The protein may further comprise a sub-cellular localization signal or a cell penetrating domain. Alternatively, or in addition, the protein may further comprise an affinity tag at either the N-terminus or the C-terminus of the protein and optionally a protease cleavage site at the proximal end of the affinity tag.

In an exemplary embodiment, the protein comprises (a) an scFv of Section I, (b) an inorganic fluorescent probe, and (c) a flexible linker connecting the scFv and the probe. The protein may further comprise a sub-cellular localization signal or a cell penetrating domain. Alternatively, or in addition, the protein may further comprise an affinity tag at either the N-terminus or the C-terminus of the protein and optionally a protease cleavage site at the proximal end of the affinity tag.

In another exemplary embodiment, the protein comprises (a) an scFv of Section I, and (b) a protein of interest, and (c) a peptide linker of 1 to about 30 amino acids connecting the scFv and the protein of interest. The protein may further comprise a sub-cellular localization signal or a cell penetrating domain. Alternatively, or in addition, the protein may further comprise an affinity tag at either the N-terminus or the C-terminus of the protein and optionally a protease cleavage site at the proximal end of the affinity tag.

In another exemplary embodiment, the protein comprises (a) an scFv of Section I, and (b) a drug (e.g., a biological product or a chemically synthesized drug), and (c) a flexible linker connecting the scFv and the drug. The protein may further comprise a sub-cellular localization signal or a cell penetrating domain. Alternatively, or in addition, the protein may further comprise an affinity tag at either the N-terminus or the C-terminus of the protein and optionally a protease cleavage site at the proximal end of the affinity tag.

In another exemplary embodiment, the protein comprises (a) an scFv of Section I, and (b) a toxin, and (c) a flexible linker connecting the scFv and the toxin. The protein may further comprise a sub-cellular localization signal or a cell penetrating domain. Alternatively, or in addition, the protein may further comprise an affinity tag at either the N-terminus or the C-terminus of the protein and optionally a protease cleavage site at the proximal end of the affinity tag.

### IV. Expression Constructs

In another aspect, the present disclosure provides nucleic acids encoding an scFv of Section I or a protein of Section III. Nucleic acid sequences encoding an scFv of Section I or a protein of Section III can be readily determined by one of skill in the art from the amino acid sequences disclosed herein. The nucleic acid can be RNA or DNA. In one embodiment, the nucleic acid is mRNA. The mRNA can be 5′ capped and/or 3′ polyadenylated. In another embodiment, the nucleic acid encoding is DNA. The DNA can be present in a vector (see below).

The nucleic acid encoding an scFv of Section I or a protein of Section III can be codon optimized for efficient translation into protein in the eukaryotic cell or animal of interest. For example, codons can be optimized for expression in humans, mice, rats, hamsters, cows, pigs, cats, dogs, fish, amphibians, plants, yeast, insects, and so forth. Programs for codon optimization are available as freeware. Commercial codon optimization programs are also available

In some embodiments, DNA encoding an scFv of Section I or a protein of Section III is operably linked to at least one promoter control sequence. In some iterations, the DNA coding sequence is operably linked to a promoter control sequence for expression in a eukaryotic cell of interest. The promoter control sequence can be constitutive or regulated. Suitable constitutive promoter control sequences include, but are not limited to, cytomegalovirus immediate early promoter (CMV), simian virus (SV40) promoter, human elongation factor-1 alpha (EF-1 alpha) promoter, adenovirus major late promoter, Rous sarcoma virus (RSV) promoter, mouse mammary tumor virus (MMTV) promoter, phosphoglycerate kinase (PGK) promoter, elongation factor (EDI)-alpha promoter, ubiquitin promoters, actin promoters, tubulin promoters, immunoglobulin promoters, fragments thereof, or combinations of any of the foregoing. Examples of suitable regulated promoter control sequences include without limit those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, FIt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter. The promoter sequence can be wild type or it can be modified for more efficient or efficacious expression. The promoter sequence can be wild type or it can be modified for more efficient or efficacious expression.

In certain embodiments, the DNA encoding an scFv of Section I or a protein of Section III is operably linked to a promoter sequence that is recognized by a phage RNA polymerase for in vitro mRNA synthesis. For example, the promoter sequence may be a T7, T3, or SP6 promoter sequence or a variation of a T7, T3, or SP6 promoter sequence.

In alternate embodiments, the DNA encoding an scFv of Section I or a protein of Section III is operably linked to a promoter sequence for in vivo expression of the scFv or protein in bacterial or eukaryotic cells. In further embodiments, the expressed scFv or protein may be purified or may be used for live cell imaging. Suitable bacterial promoters include, without limit, T7 promoters, lac operon promoters, trp promoters, variations thereof, and combinations thereof. An exemplary bacterial promoter is tac which is a hybrid of trp and lac promoters. Non-limiting examples of suitable eukaryotic promoters are listed above.

In additional aspects, DNA encoding an scFv of Section I or a protein of Section II may be linked to a polyadenylation signal (e.g., SV40 polyA signal, bovine growth hormone (BGH) polyA signal, etc.) and/or at least one transcriptional termination sequence. Additionally, the DNA encoding an scFv of Section I or a protein of Section III also may be linked to a sequence encoding at least one nuclear localization signal or at least one cell-penetrating domain.

In various embodiments, the DNA sequence encoding an scFv of Section I or a protein of Section III may be present in a vector. Suitable vectors include plasmid vectors, phagemids, cosmids, artificial/mini-chromosomes, transposons, and viral vectors. In one embodiment, the vector is a plasmid vector. Non-limiting examples of suitable plasmid vectors include pUC, pBR322, pET, pBluescript, and variants thereof. In another embodiment, the vector is a viral vector. Non-limiting examples of suitable viral vectors include lentiviral vectors, adeno-associated viral vectors, adenovirus vectors, alphavirus vectors, herpesvirus vectors, and vaccinia virus vectors. In such embodiments, the expressed viral vector can be purified for use in the methods detailed below in Section V. In the above embodiments, the vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

In some embodiments, the expression vector comprising the DNA sequence encoding the protein is operably linked to at least one transcriptional control sequence for expression of the construct in a cell of interest. For example, DNA encoding the construct can be operably linked to a promoter sequence that is recognized by RNA polymerase III (Pol III). Examples of suitable Pol III promoters include, but are not limited to, mammalian U6, U3, H1, and 7SL RNA promoters.

Exemplary expression constructs are listed in Table 4, the sequences of which can be found at Addgene (the nonprofit plasmid repository). The design of these constructs is not limiting. Constructs similar, modified, or equivalent to those described herein can be used in the practice of the embodiments of the present invention without undue experimentation.

### V. Method for Imaging

Another aspect of the present disclosure encompasses use of scFv's of Section I for live cell imaging. As used herein, the term “live cell imaging” refers generally to the study of living cells and encompasses a variety of applications that visualize and/or quantify a molecule within a cell, a multi-cellular structure, an embryo, an organ, or a whole animal. Non-limiting examples of live cell imaging applications include visualizing and/or quantifying protein co-localization; 3D imaging of live cells, tissues, model organisms, and small animals; biosensing and protein-protein interactions; visualizing and/or quantifying protein diffusion and kinetics; single molecule tracking; visualizing and/or quantifying co-translational dynamics of nascent peptide chains; visualizing and/or quantifying dynamics of short-lived proteins (e.g., transcription factors, etc.); and tracking the spatiotemporal dynamics of post-translational modifications and protein conformational changes.

The method comprises providing a protein comprising an scFv and a tag, and a cell comprising an epitope to which the scFv specifically binds; labeling the cell with the protein; and imaging the cell to detect and optionally quantify the tag. In some embodiments, the scFv specifically binds an epitope that is endogenous to the cell. In other embodiments, the scFv specifically binds an exogenous epitope to the cell. In these embodiments, the cell is engineered to at least one copy of the exogenous epitope to which the scFv specifically binds, typically as a fusion protein. The number of copies may be optimized as needed (e.g., to maximize the detectable signal, etc.). For instance, in one example, the cell is engineered to express a fusion protein comprising at least two copies of an epitope to which the scFv specifically binds. In another example, the cell is engineered to express a fusion protein comprising at least five copies of an epitope to which the scFv specifically binds. In another example, the cell is engineered to express a fusion protein comprising at least ten copies of an epitope to which the scFv specifically binds. In another example, the cell is engineered to express a fusion protein comprising 1 to 500 copies of an epitope to which the scFv specifically binds. In another example, the cell is engineered to express a fusion protein comprising 1 to 400 copies of an epitope to which the scFv specifically binds. In another example, the cell is engineered to express a fusion protein comprising 1 to 300 copies of an epitope to which the scFv specifically binds. In another example, the cell is engineered to express a fusion protein comprising 1 to 200 copies of an epitope to which the scFv specifically binds. In another example, the cell is engineered to express a fusion protein comprising 1 to 100 copies of an epitope to which the scFv specifically binds. In another example, the cell is engineered to express a fusion protein comprising 1 to 50 copies of an epitope to which the scFv specifically binds. In another example, the cell is engineered to express a fusion protein comprising 1 to 30 copies of an epitope to which the scFv specifically binds. In another example, the cell is engineered to express a fusion protein comprising 20 to 30 copies of an epitope to which the scFv specifically binds. In another example, the cell is engineered to express a fusion protein comprising 1 to 20 copies of an epitope to which the scFv specifically binds. In another example, the cell is engineered to express a fusion protein comprising 10 to 20 copies of an epitope to which the scFv specifically binds. In another example, the cell is engineered to express a fusion protein comprising 1 to 10 copies of an epitope to which the scFv specifically binds. In another example, the cell is engineered to express a fusion protein comprising 1 to 5 copies of an epitope to which the scFv specifically binds. In exemplary embodiments, the epitope comprises SEQ ID NO: 22. In other exemplary embodiments, the epitope comprises SEQ ID NO: 23. In other exemplary embodiments, the epitope comprises SEQ ID NO: 24. In other exemplary embodiments, the epitope comprises SEQ ID NO: 48. In other exemplary embodiments, the epitope comprises SEQ ID NO: 49.

Proteins comprising an scFv and a tag are described in Section III. Typically the protein comprises an scFv of Section I, an imaging agent, and a linker connecting the scFv and the imaging agent. The imaging agent may be at the N-terminus or C-terminus of the protein. Preferred imaging agents include, but are not limited to, fluorescent proteins, bioluminescent proteins, and inorganic fluorescent probes, self-labeling tags. In some embodiments, a protein may further comprise one or more additional tag and/or a sub-cellular localization signal and/or a cell-penetrating domain.

A variety of cell types are suitable for use in the method, including prokaryotic cells, eukaryotic cells, and archaeal cells. For example, the cell can be a human cell, a non-human mammalian cell, a non-mammalian vertebrate cell, an invertebrate cell, an insect cell, a plant cell, a yeast cell, a single cell eukaryotic organism, a bacterial cell, or an archaeal cell. Suitable eukaryotic cells may be primary cells, or cells from an immortalized cell line. An extensive list of cell lines may be found in the American Type Culture Collection catalog (ATCC, Manassas, Va.). Exemplary primary cells include but are not limited to fibroblasts, epithelial cells, endothelial cells, stem cells, neurons, kidney cells, liver cells, lung cells, pancreatic cells, cardiomyocytes, immune cells, cone cells, rod cells, and the like. The cells may be isolated cells or part of multicellular structure, including but not limited to a tissue, an embryo, an organ, or a whole animal.

Cells are preferably labeled with a protein comprising an scFv and a tag by transiently or stably transfecting the cell with an expression construct encoding the protein. In embodiments where the cell is engineered to express an epitope to which the scFv specifically binds, the expression construct encoding the protein may also encode a fusion protein comprising the epitope. Alternatively, the fusion protein comprising the epitope may be encoded by a second expression construct. Cells can also be labeled with purified protein. See, for example, McNeil et al., J. Cell Sci. 88, 669-678 (1987). Other methods are also known in the art.

A variety of live cell imaging techniques are known in the art and suitable for use in the method. The choice of an appropriate technique will be influenced, in part, by the choice of the tag and the cell type. Non-limiting examples of live cell imaging techniques include transmission light microscopy (e.g., bright field, dark field, phase contrast, differential interference contrast, etc.), fluorescence microscopy, confocal microscopy, multiphoton microscopy, total internal reflection fluorescence microscopy, fluorescence lifetime imaging microscopy, forster resonance energy transfer microscopy, BRET imaging, fluorescence correlation spectroscopy, single-molecule tracking, photo-activation light microscopy, and light sheet microscopy.

### VI. Kits

The present disclosure also encompasses kits for imaging cells. In some embodiments, the kit comprises a purified protein of Section II. In some embodiments, the kit comprises an expression construct of Section IV. In some embodiments, the kit comprises a cell stably or transiently transfected with an expression construct of Section IV, such that the cell expresses at least one protein of Section III. The cell may be a mammalian cell. Preferably, the cell is a human cell. The human cell may be a cell line cell chosen from a human U2OS cell, a human MCF10A, a human SKOV3, or a human iPS. The protein may comprise an scFv that specifically binds a target comprising an epitope selected from SEQ ID NO: 21, SEQ ID NO: 22, SEQ ID NO: 23, SEQ ID NO: 24, SEQ ID NO: 48, or SEQ ID NO: 49. The protein may also comprise an scFv that has HVRs derived from the antibody F11.2.32 (see, e.g., Protein Database Bank ID 2HRP), the antibody REGN1909 (see, e.g., Protein Database Bank ID 5VYF), the antibody 12CA5 (Protein Database Bank ID 2HRP), an antibody listed in Table A, or from SEQ ID NOs: 42, 43, 44, 45, 46 and 47. Amino acid sequences for the antibodies identified by a PDB ID can be obtained from Protein Database Bank, and the HVRs identified as described herein. In preferred embodiments, protein further comprises an imaging agent and a linker connecting the scFv and the imaging agent. The imaging agent may be at the N-terminus or C-terminus of the protein. Preferred imaging agents include, but are not limited to, fluorescent proteins, bioluminescent proteins, inorganic fluorescent probes, and self-labeling tags. In some embodiments, a protein may further comprise one or more additional tag and/or a sub-cellular localization signal and/or a cell-penetrating domain.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

The following references are cited in the Examples.

1. Chalfie, M. GFP: Lighting up life. Proc. Natl. Acad. Sci. U.S.A. 106, 10073-10080 (2009).

2. Tsien, R. Y. THE GREEN FLUORESCENT PROTEIN. Annu. Rev. Biochem. 67, 509-544 (1998).

3. Morisaki, T. & Stasevich, T. J. Quantifying Single mRNA Translation Kinetics in Living Cells. Cold Spring Harb. Perspect. Biol. 10, a032078 (2018).

4. Lyon, K. & Stasevich, T. J. Imaging Translational and Post-Translational Gene Regulatory Dynamics in Living Cells with Antibody-Based Probes. Trends Genet. 33, 322-335 (2017).

5. Bothma, J. P., Norstad, M. R., Alamos, S. & Garcia, H. G. LlamaTags: A Versatile Tool to Image Transcription Factor Dynamics in Live Embryos. Cell 173, 1810-1822.e16 (2018).

6. Stasevich, T. J. et al. Regulation of RNA polymerase II activation by histone acetylation in single living cells. Nature 516, 272-275 (2014).

7. Stoeber, M. et al. A Genetically Encoded Biosensor Reveals Location Bias of Opioid Drug Action. Neuron 98, 963-976.e5 (2018).

8. Holliger, P. & Hudson, P. J. Engineered antibody fragments and the rise of single domains. Nat. Biotechnol. 23, 1126-1136 (2005).

9. Porter, R. R. The Hydrolysis of Rabbit y-Globulin and Antibodies with Crystalline Papain. Biochem J73, 119-127 (1959).

10. Skerra, A. & Pluckthun, A. Assembly of a functional immunoglobulin Fv fragment in Escherichia coli. Science 240, 1038-1041 (1988).

11. Inbar, D., Hochman, J. & Givol, D. Localization of Antibody-Combining Sites within the Variable Portions of Heavy and Light Chains. Proc. Natl. Acad. Sci. 69, 2659-2662 (1972).

12. Schaefer, J. V., Honegger, A. & Plückthun, A. Construction of scFv Fragments from Hybridoma or Spleen Cells by PCR Assembly. in Antibody Engineering (eds. Kontermann, R. & Dübel, S.) 21-44 (Springer Berlin Heidelberg, 2010). doi:10.1007/978-3-642-01144-3_3

13. Ries, J., Kaplan, C., Platonova, E., Eghlidi, H. & Ewers, H. A simple, versatile method for GFP-based super-resolution microscopy via nanobodies. Nat. Methods 9, 582-584 (2012).

14. Kirchhofer, A. et al. Modulation of protein properties in living cells using nanobodies. Nat. Struct. Mol. Biol. 17, 133-138 (2010).

15. Virant, D. et al. A peptide tag-specific nanobody enables high-quality labeling for dSTORM imaging. Nat. Commun. 9, (2018).

16. Hamers-Casterman, C. et al. Naturally occurring antibodies devoid of light chains. Nature 363, 446-448 (1993).

17. Morisaki, T. et al. Real-time quantification of single RNA translation dynamics in living cells. Science 352, 1425-1429 (2016).

18. Yan, X., Hoek, T. A., Vale, R. D. & Tanenbaum, M. E. Dynamics of Translation of Single mRNA Molecules In Vivo. Cell 165, 976-989 (2016).

19. Wang, C., Han, B., Zhou, R. & Zhuang, X. Real-Time Imaging of Translation on Single mRNA Transcripts in Live Cells. Cell 165, 990-1001 (2016).

20. Wu, B., Eliscovich, C., Yoon, Y. J. & Singer, R. H. Translation dynamics of single mRNAs in live cells and neurons. Science 352, 1430-1435 (2016).

21. Pichon, X. et al. Visualization of single endogenous polysomes reveals the dynamics of translation in live human cells. J Cell Biol 214, 769-781 (2016).

22. Viswanathan, S. et al. High-performance probes for light and electron microscopy. Nat. Methods 12, 568-576 (2015).

23. Tanenbaum, M. E., Gilbert, L. A., Qi, L. S., Weissman, J. S. & Vale, R. D. A Protein-Tagging System for Signal Amplification in Gene Expression and Fluorescence Imaging. Cell 159, 635-646 (2014).

24. Kimura, H., Hayashi-Takanaka, Y., Stasevich, T. J. & Sato, Y. Visualizing posttranslational and epigenetic modifications of endogenous proteins in vivo. Histochem. Cell Biol. 144, 101-109 (2015).

25. Hayashi-Takanaka, Y. et al. Tracking epigenetic histone modifications in single cells using Fab-based live endogenous modification labeling. Nucleic Acids Res. 39, 6475-6488 (2011).

26. McNeil, P. L. & Warder, E. Glass beads load macromolecules into living cells. J. Cell Sci. 88, 669-678 (1987).

27. Tanaka, T. & Rabbitts, T. H. Protocol for the selection of single-domain antibody fragments by third generation intracellular antibody capture. Nat. Protoc. 5, 67-92 (2010).

28. Visintin, M. et al. The intracellular antibody capture technology (IACT): towards a consensus sequence for intracellular antibodies. J. Mol. Biol. 317, 73-83 (2002).

29. Keller, B.-M. et al. A Strategy to Optimize the Generation of Stable Chromobody Cell Lines for Visualization and Quantification of Endogenous Proteins in Living Cells. Antibodies 8, 10 (2019).

30. Voigt, F. et al. Single-Molecule Quantification of Translation-Dependent Association of mRNAs with the Endoplasmic Reticulum. Cell Rep. 21, 3740-3753 (2017).

31. Horvathova, I. et al. The Dynamics of mRNA Turnover Revealed by Single-Molecule Imaging in Single Cells. Mol. Cell 68, 615-625.e9 (2017).

32. Hanes, J., Jermutus, L., Weber-Bornhauser, S., Bosshard, H. R. & Pluckthun, A. Ribosome display efficiently selects and evolves high-affinity antibodies in vitro from immune libraries. Proc. Natl. Acad. Sci. 95, 14130-14135 (1998).

33. Wörn, A. et al. Correlation between in Vitro Stability and in Vivo Performance of Anti-GCN4 Intrabodies as Cytoplasmic Inhibitors. J. Biol. Chem. 275, 2795-2803 (2000).

34. Ewert, S., Honegger, A. & Plückthun, A. Stability improvement of antibodies for extracellular and intracellular applications: CDR grafting to stable frameworks and structure-based framework engineering. Methods 34, 184-199 (2004).

35. Green, N. et al. Immunogenic structure of the influenza virus hemagglutinin. Cell 28, 477-487 (1982).

36. Wongso, D., Dong, J., Ueda, H. & Kitaguchi, T. Flashbody: A Next Generation Fluobody with Fluorescence Intensity Enhanced by Antigen Binding. Anal. Chem. 89, 6719-6725 (2017).

37. Fujiwara, K. et al. A Single-Chain Antibody/Epitope System for Functional Analysis of Protein-Protein Interactions †. Biochemistry 41, 12729-12738 (2002).

38. Sato, Y. et al. A Genetically Encoded Probe for Live-Cell Imaging of H4K20 Monomethylation. J. Mol. Biol. 428, 3885-3902 (2016).

39. Sato, Y. et al. Genetically encoded system to track histone modification in vivo. Sci. Rep. 3, 2436 (2013).

40. O'Connell, K. M. S. Kv2.1 Potassium Channels Are Retained within Dynamic Cell Surface Microdomains That Are Defined by a Perimeter Fence. J. Neurosci. 26, 9609-9618 (2006).

41. Colca, J. R. et al. Identification of a novel mitochondrial protein (′mitoNEET) cross-linked specifically by a thiazolidinedione photoprobe. Am. J. Physiol. EndocrinoL Metab. 286, E252-260 (2004).

42. Los, G. V. et al. HaloTag: A Novel Protein Labeling Technology for Cell Imaging and Protein Analysis. ACS Chem. Biol. 3, 373-382 (2008).

43. Keppler, A. et al. A general method for the covalent labeling of fusion proteins with small molecules in vivo. Nat. Biotechnol. 21, 86-89 (2003).

44. Kimura, H. & Cook, P. R. Kinetics of Core Histones in Living Human Cells Little Exchange of H3 and H4 and Some Rapid Exchange of H2b. J. Cell Biol. 153, 1341-1354 (2001).

45. Carlini, L., Benke, A., Reymond, L., Lukinaviĉius, G. & Manley, S. Reduced Dyes Enhance Single-Molecule Localization Density for Live Superresolution Imaging. ChemPhysChem 15, 750-755 (2014).

46. Mazza, D., Abernathy, A., Golob, N., Morisaki, T. & McNally, J. G. A benchmark for chromatin binding measurements in live cells. Nucleic Acids Res. 40, e119-e119 (2012).

47. Nozaki, T. et al. Dynamic Organization of Chromatin Domains Revealed by Super-Resolution Live-Cell Imaging. Mol. Cell 67, 282-293.e7 (2017).

48. Grimm, J. B. et al. A general method to improve fluorophores for live-cell and single-molecule microscopy. Nat. Methods 12, 244-250 (2015).

49. Miller, S. et al. Disruption of Dendritic Translation of CaMKIIα Impairs Stabilization of Synaptic Plasticity and Memory Consolidation. Neuron 36, 507-519 (2002).

50. Baroux, C., Autran, D., Gillmor, C. S., Grimanelli, D. & Grossniklaus, U. The Maternal to Zygotic Transition in Animals and Plants. Cold Spring Harb. Symp. Quant. Biol. 73, 89-100 (2008).

51. Martin, K. C. & Zukin, R. S. RNA Trafficking and Local Protein Synthesis in Dendrites: An Overview. J. Neurosci. 26, 7131-7134 (2006).

52. Hirokawa, N. mRNA transport in dendrites: RNA granules, motors, and tracks. J. Neurosci. Off. J. Soc. Neurosci. 26, 7139-7142 (2006).

53. Halstead, J. M. et al. Translation. An RNA biosensor for imaging the first round of translation from single cells to living animals. Science 347, 1367-1671 (2015).

54. Packer, M. S. & Liu, D. R. Methods for the directed evolution of proteins. Nat. Rev. Genet. 16,379-394 (2015).

55. McCafferty, J., Griffiths, A. D., Winter, G. & Chiswell, D. J. Phage antibodies: filamentous phage displaying antibody variable domains. Nature 348, 552-554 (1990).

56. Muyldermans, S. et al. Camelid immunoglobulins and nanobody technology. Vet. Immunol. Immunopathol. 128,178-183 (2009).

57. Gao, J. et al. Affibody-based nanoprobes for HER2-expressing cell and tumor imaging. Biomaterials 32,2141-2148 (2011).

58. Sha, F., Salzman, G., Gupta, A. & Koide, S. Monobodies and other synthetic binding proteins for expanding protein science. Protein Sci. 26,910-924 (2017).

59. Boersma, S. et al. Multi-color single molecule imaging uncovers extensive heterogeneity in mRNA decoding. bioRxiv (2018). doi:10.1101/477661.

60. Johnson, B. et al. Kv2 potassium channels form endoplasmic reticulum/plasma membrane junctions via interaction with VAPA and VAPB. Proc. Natl. Acad. Sci. 115, E7331-E7340 (2018).

61. Zhao, N., Schmitt, M. A. & Fisk, J. D. Phage display selection of tight specific binding variants from a hyperthermostable Sso7d scaffold protein library. FEBS J. 283,1351-1367 (2016).

62. Zhao, N., Spencer, J., Schmitt, M. A. & Fisk, J. D. Hyperthermostable binding molecules on phage: Assay components for point-of-care diagnostics for active tuberculosis infection. Anal. Biochem. 521,59-71 (2017).

63. Schindelin, J. et al. Fiji: an open-source platform for biological-image analysis. Nat. Methods 9,676-682 (2012).

64. Thevenaz, P., Ruttimann, U. E. & Unser, M. A pyramid approach to subpixel registration based on intensity. IEEE Trans. Image Process. 7, 27-41 (1998).

65. Koulouras, G. et al. EasyFRAP-web: a web-based tool for the analysis of fluorescence recovery after photobleaching data. Nucleic Acids Res. 46, W467-W472 (2018).

66. Tokunaga, M., Imamoto, N. & Sakata-Sogawa, K. Highly inclined thin illumination enables clear single-molecule imaging in cells. Nat. Methods 5, 159-161 (2008).

67. Edelstein, A. D. et al. Advanced methods of microscope control using μManager software. J. Biol. Methods 1, e10 (2014).

68. Tinevez, J.-Y. et al. TrackMate: An open and extensible platform for single-particle tracking. Methods 115,80-90 (2017).

69. Yamagata, K. et al. Noninvasive visualization of molecular events in the mammalian zygote. Genes. N. Y. N 2000 43,71-79 (2005).

### Example 1

This example describes the development of a new strategy for developing scFv's for live cell imaging that addresses the deficiencies in the field, some of which are described in the Background Section. To bypass many of the difficulties associated with probe development, a diverse set of scFv scaffolds that have already been proven to fold properly and function within the reduced cytoplasm of living cells were used as a starting point. Onto these scaffolds, all six CDRs from an epitope-specific antibody³⁴ were loop grafted. Depending on the compatibility of the scaffold and CDRs, this produces a hybrid scFv that retains the folding stability of the scaffold, while acquiring the binding specificity of the grafted CDRs.

To demonstrate the approach, two new hybrid scFvs were generated that bind to the classic linear HA epitope (SEQ ID NO: 23)³⁵ in vivo. One of these hybrid scFvs, referred to as “HA frankenbody,” was used to label in multiple colors a variety of proteins in diverse live-cell environments, including nuclear, cytoplasmic, membrane, and mitochondrial proteins tagged on either the N- or C-terminus with between one and ten HA epitopes. To further highlight the versatility of the HA frankenbody, three novel experiments were performed. First, single 1×HA-tagged histones were tracked throughout the nucleus of living cells to generate dense and super-resolved chromatin mobility maps. Second, single mRNA translation dynamics were tracked in two colors simultaneously to examine how mRNA sequence and the local cellular environment impact translation site localization and mobility. Third, the HA frankenbody was used to image HA-tagged proteins in developing zebrafish embryos, proving its utility in living model organisms. Together, these diverse applications demonstrate the remarkable versatility of the HA frankenbody for studying complex protein dynamics in living systems with high spatiotemporal resolution. It is therefore anticipated the HA frankenbody will be a powerful new tool for live-cell imaging.

Design Strategy and Initial Screening of Frankenbodies:

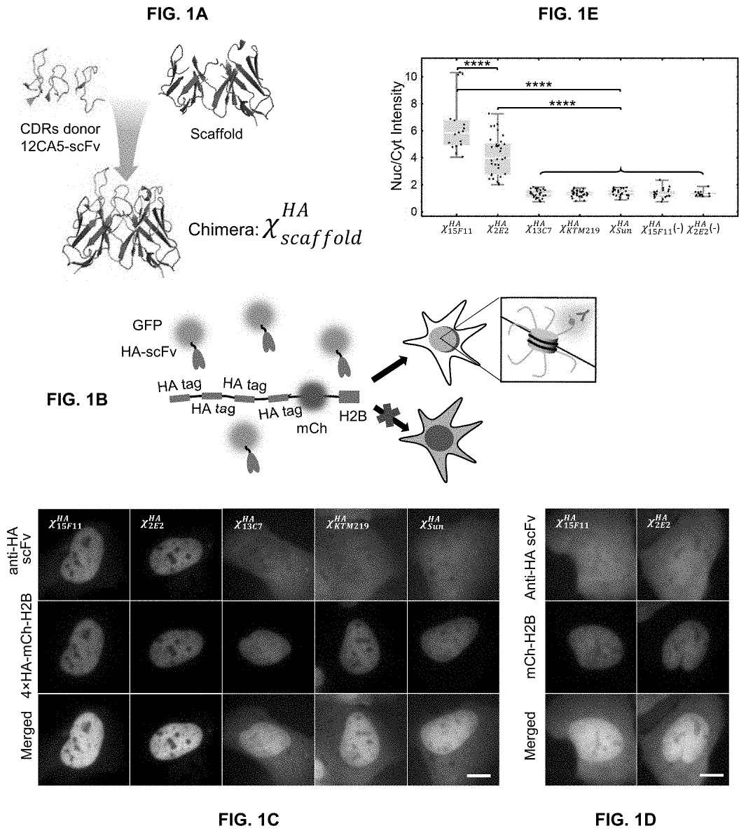

The HA-frankenbody was engineered from six complementarity determining regions (CDRs, or loops) within the heavy and light chains of a published anti-HA scFv (parental full-length antibody: 12CA5; anti-HA scFV: 12CA5-scFv)^{36,37} (FIG. 1A). On its own, this wildtype anti-HA scFv (12CA5-scFv) does not fold properly in the reduced intracellular environment, and therefore displays little to no affinity for HA epitopes in living human U2OS cells³⁶. It was discovered this folding issue could be addressed by grafting the CDRs onto more stable and sequence similar scFv scaffolds (FIG. 1A). To test this, five scFv scaffolds that have already been successfully used for live-cell imaging purposes, and that have a wide range of sequence identity compared to wtHA-scFv, were used. In particular, the sequences of the heavy chain variable regions (VH) were 47-89% identical, while the sequences of the light chain variable regions (VL) were 50-67% identical (Table 1). The five scaffolds chosen included (1) an scFv that specifically binds histone H4 mono-methylated at Lysine 20 (H4K20me; 15F11)³⁸; (2) an H3K9ac specific scFv (13C7)³⁹; (3) an H420me2-specific scFv (2E2, unpublished); (4) a SunTag-specific scFv²³; and (5) a bone Gla protein (BGP) specific scFv (KTM219)³⁶. Among these scaffolds, 15F11 and 2E2 have the greatest sequence identity compared to the wildtype 12CA5-scFv (FIG. 1B). It was hypothesized there would be a higher chance of grafting success with either of these scaffolds.

To test the hypothesis, the anti-HA scFv CDR loops of 12CA5-scFv were grafted onto the five chosen scFv scaffolds. The resulting five chimeric scFvs are referred to using the following nomenclature: χ_{scaffold}^{HA}. For example, χ_{15F11}^{HA} specifies the chimeric scFv that was generated by loop grafting the anti-HA CDRs onto the 15F11 scFv scaffold. To screen the chimeras, each was fused with the green fluorescent protein mEGFP and each of the resulting plasmids co-transfected into U2OS cells, together with a plasmid encoding 4×HA-tagged histone H2B fused to the red fluorescent protein mCherry (4×HA-mCh-H2B). If a chimeric scFv binds to the HA epitope in living cells, it should co-localize with the HA-tagged H2B in the nucleus, as shown in FIG. 1B. Live-cell imaging revealed χ_{15F11}^{HA} and χ_{2E2}^{HA} were superior, displaying little to no misfolding and/or aggregation, strong expression, and excellent co-localization with H2B in the nucleus. In contrast, the other three scFvs did not show any co-localization signal (FIG. 1C and FIG. 1E). Moreover, in control cells lacking HA tags, both χ_{15F11}^{HA} and χ_{2E2}^{HA} displayed uniform expression (FIG. 1D and FIG. 1E), indicative of free diffusion without non-specific binding. According to our screen, both χ_{15F11}^{HA} and χ_{2E2}^{HA} function well in living cells, although χ_{15F11}^{HA} labels HA tags slightly better than χ_{2E2}^{HA} (FIG. 1E). The χ_{15F11}^{HA} variant was chosen for additional screening, which is referred to in this example as the “HA frankenbody” due to its construction via grafting.

Multicolor Labeling of HA-Tagged Proteins in Diverse Intracellular Environments:

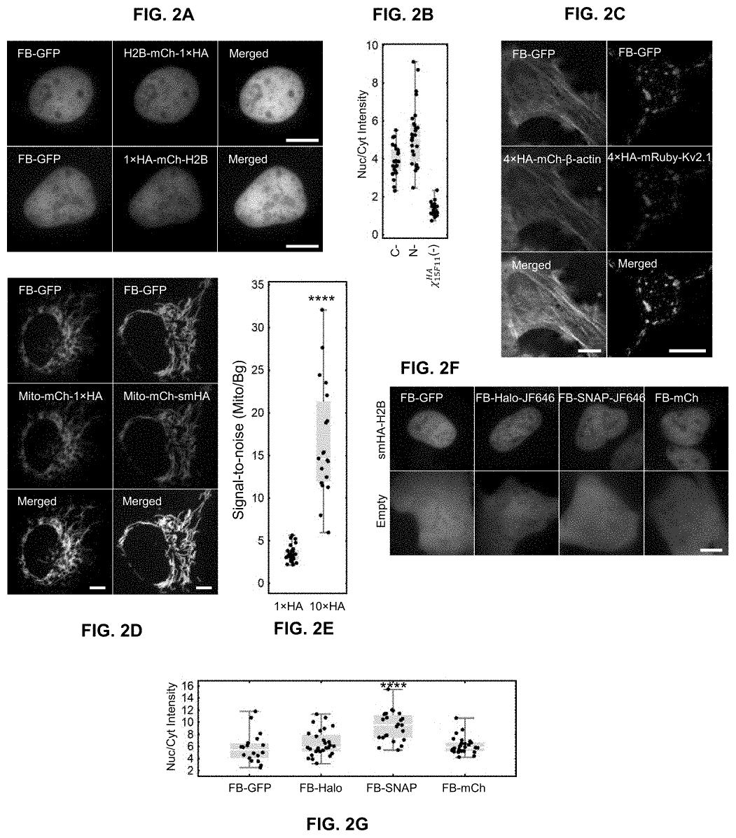

The HA frankenbody was tested in a variety of different settings. First, since the initial screen had been done with a 4×HA tag, the HA frankenbody was tested to see if it could also bind a 1×HA tag placed on either end of a POI. To test this, two plasmids were constructed: 1×HA fused to the C-terminus of H2B-mCherry (H2B-mCh-1×HA) and 1×HA fused to the N-terminus of mCherry-H2B (1×HA-mCh-H2B). In both cases, the HA frankenbody displayed strong nuclear localization (FIG. 2A). Beyond nuclear proteins, the HA frankenbody were tested in the cell cytoplasm, another reducing environment that can interfere with intradomain disulfide bond formation³³. This was tested by creating a new target plasmid encoding the cytoplasmic protein β-actin fused with a 4×HA-tag and mCherry (4×HA-mCh-β-actin). When this plasmid was expressed in cells, co-expressed frankenbodies again took on the distinct localization pattern of their targets, in this case colocalizing with 4×HA-mCh-β-actin along filamentous actin fibers (FIG. 2B, left). It was therefore concluded that both nuclear and cytoplasmic HA-tagged proteins can be selectively labeled with high efficiency by the HA frankenbody in living human cells.

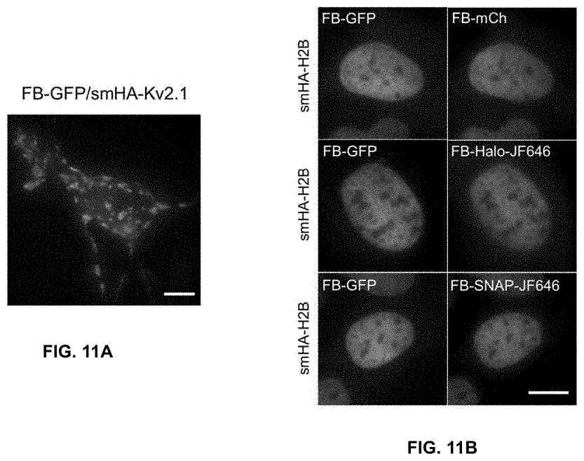

To test if frankenbodies could also work in more sensitive cell types, living primary rat cortical neurons were co-transfected with the HA frankenbody (FB-GFP) and a 4×HA-tagged transmembrane protein Kv2.1 (4×HA-mRuby-Kv2.1). In cortical and hippocampal neurons, Kv2.1 has been demonstrated to be localized to the plasma membrane, where it forms large (up to one micron in diameter) cell-surface clusters, providing a distinct localization pattern⁴⁰. In cells expressing FB-GFP and 4×HA-mRuby-Kv2.1, the HA frankenbody again took on the distinct localization pattern of its target (FIG. 2B right). In addition, the distinctive pattern could be seen for over a week after transient transfection of FB-GFP and smHA-Kv2.1 (FIG. 11A). This demonstrates the HA frankenbody can selectively bind membrane proteins as well as cytoplasmic and nuclear proteins, depending on the presence of the HA tag. This also demonstrates continual expression of frankenbody does not detrimentally impact sensitive cells and, moreover, frankenbodies have exceptionally long half-lives when bound to their targets.

Because the HA epitope is so small, it can be repeated many times within tags to increase signal-to-noise. The HA spaghetti-monster tag (smHA), for example, contains 10 HA epitopes and has been used to amplify fluorescence signal from tagged proteins²². To test how well the frankenbody labels the HA spaghetti monster tag, cells were co-transfected with a plasmid encoding either a 1×HA or smHA fused to the C-terminus of an mCherry-tagged mitochondrial protein, mitoNEET⁴¹ (referred to “Mito”). As before, HA frankenbody colocalized well with target proteins (FIG. 2C and FIG. 2D). Moreover, quantification revealed the mitochondrial signal-to-noise (Mito/Bg) was on average 4.7±0.5 (Mean±SEM) times higher for smHA-tagged Mito compared to 1×HA-tagged Mito. Thus, in combination with repeat-epitope tags, the HA frankenbody can be used to amplify fluorescence in live-cell imaging applications.

Finally, to ensure the HA frankenbody is as broadly applicable as possible, it was tested to determine if it could tolerate different fluorescent protein fusion partners that might be needed in multicolor imaging applications. GFP and its derivatives are generally superior fusion partners because their high stability actually helps stabilize and solubilize the tagged protein. This was observed, for example, during the development of the SunTag scFv²³. To test how well the HA frankenbody tolerates different tags, it was fused to mCherry, HaloTag⁴² and SNAP-tag⁴³. Encouragingly, all three frankenbody constructs co-localized with HA-tagged H2B in the nucleus of living U2OS cells, similar or even better than the original GFP-tagged frankenbody (FIG. 2E-upper, FIG. 2F). Furthermore, when different colored versions of FB were co-expressed in cells, one color did not dominate over the others (FIG. 11B). Finally, all three constructs displayed relatively diffuse localization patterns in cells lacking the HA-tag (FIG. 2E, lower). These data indicate the HA frankenbody can indeed tolerate different fusion partners, including green (GFP), red (mCherry), and far-red (SNAP-tag/HaloTag with far-red ligands) fluorescent proteins. Thus, the HA frankenbody can label HA-tagged proteins in a rainbow of colors in living cells.

Immunostaining and Western Blotting with Purified Recombinant Frankenbody:

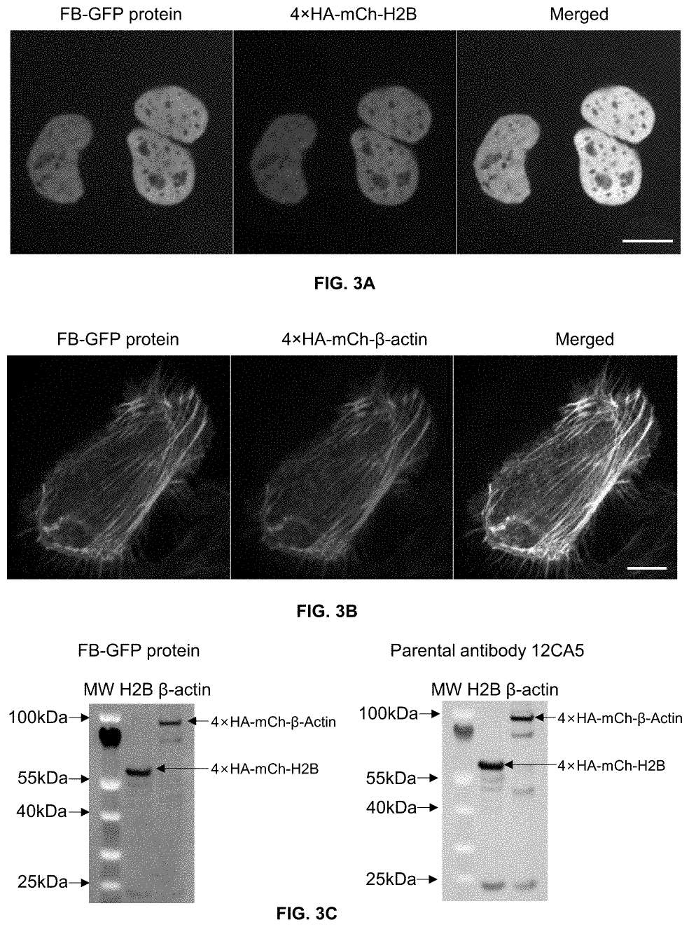

The HA frankenbody was also evaluated for its potential to replace costly anti-HA antibodies in traditional assays such as immunostaining and Western blots. To test this, the frankenbody gene fused with mEGFP and a hexahistidine tag was cloned into an E. coli expression vector, pET23b. The recombinant frankenbody was expressed and the soluble portion was purified from E. coli. Using this purified fraction, fixed cells expressing HA-tagged H2B and HA-tagged β-actin were immunostained. Similar to the observations in living cells, the purified HA frankenbody beautifully stained both the HA-tagged nuclear and cytoplasmic proteins, but now with almost no observable background signal (FIG. 3A and FIG. 3B).

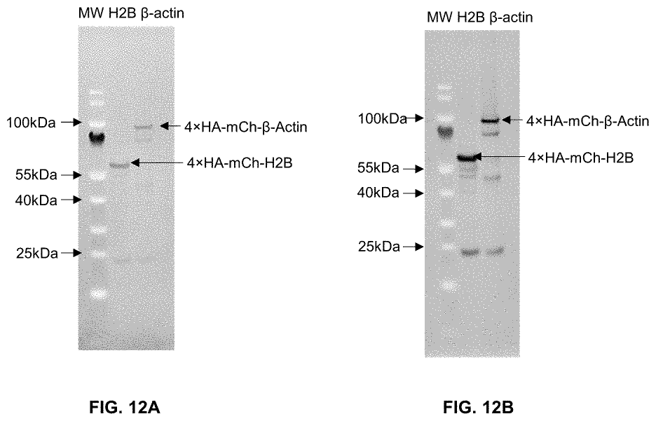

The suitability of the HA frankenbody for Western blotting was next tested. For this, U2OS cells were harvested 10 h after transiently transfecting either HA-tagged H2B or HA-tagged β-actin. In contrast to the parental 12CA5 full-length anti-HA antibody, which was stained using a secondary antibody conjugated with Alexa488, the frankenbody Western blot used the GFP signal alone for detection. Nevertheless, similar dark and sharp bands were seen on the frankenbody membrane as the 12CA5 membrane (FIG. 3C and FIG. 12). Although several of the bands were dimmer than those seen using 12CA5, the difference was attributed to signal amplification from the secondary antibody. In principle, a similar signal-to-noise could be attained using the GFP-tagged HA frankenbody with secondary antibodies against GFP. Together, the Western blot and immunostaining results strongly suggest the HA frankenbody can serve as a cost-effective replacement for full-length HA antibody in widely used in vitro applications.

HA Frankenbody Specifically Binds the HA Epitope for Minutes at a Time in Live Cells:

An ideal imaging probe binds its target with high affinity to maximize the fraction of target epitopes bound and thereby increase signal-to-noise. In general, a high bound fraction is established by a large ratio of probe:target binding off to on times; in other words, the time a probe remains bound to a target is ideally much longer than the time it takes a probe to bind a target. Although the latter depends sensitively on the concentrations of both target and probe, the former is a fixed biophysical parameter that is useful for planning and interpreting experiments. With this in mind, the length of time the HA frankenbody remains bound to the HA epitope in living cells was measured.

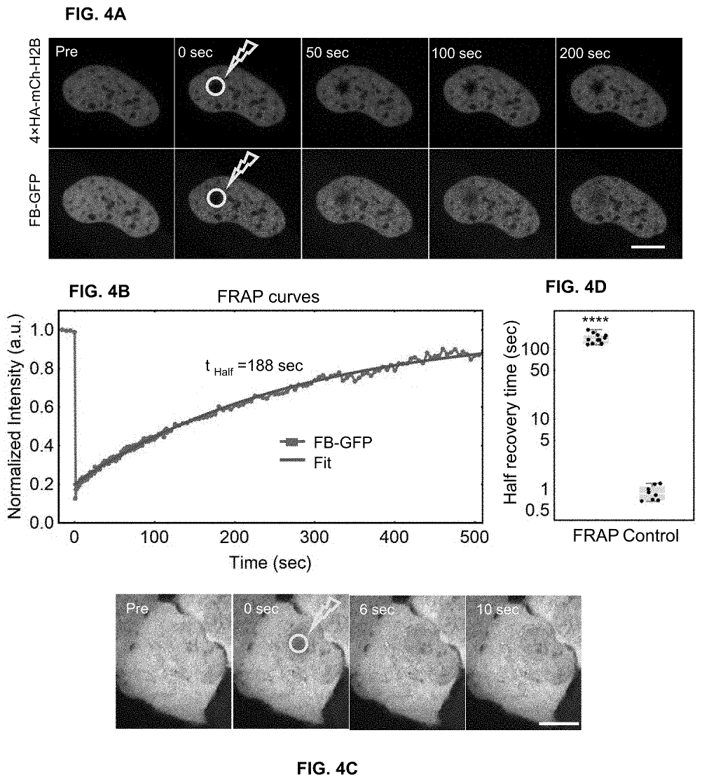

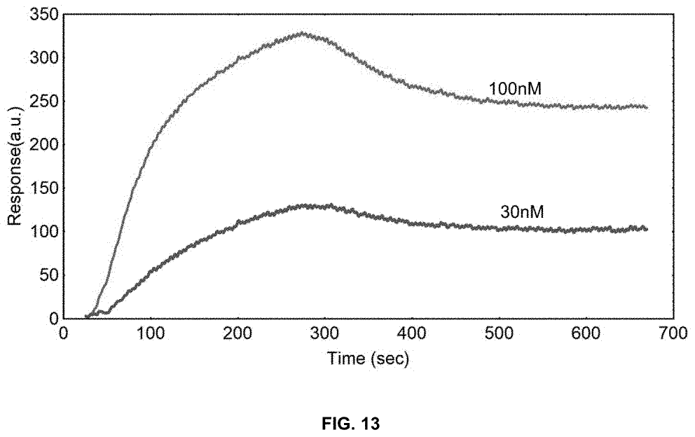

To accurately measure the binding kinetics of HA frankenbody, Fluorescence Recovery After Photobleaching (FRAP) experiments were performed in cells co-expressing GFP-tagged HA frankenbody and 4×HA-mCh-H2B. As H2B is bound to chromatin for hours at a time, any recovery on the minutes timescale can be attributed to the turnover of frankenbody alone. Consistent with this, FRAP in the red 4×HA-mCh-H2B channel displayed little or no recovery on the timescale of the experiment. In contrast, FRAP in the green FB-GFP channel slowly recovered (FIG. 4A). Quantitative analyses of FRAP curves revealed the half recovery time to be around 2˜3 minutes (FIG. 4B and FIG. 4D). As a control, FRAP experiments were repeated in cells transfected with just the HA frankenbody (i.e. lacking HA epitopes). Here, the FRAP recovery lasted just seconds, consistent with little to no non-specific binding of the HA frankenbody (FIG. 4C and FIG. 4D). It was therefore concluded that the majority of frankenbodies bind target HA epitopes for minutes at a time in living cells. This is consistent with the high binding affinity (K_{D}=14.7±7.4 nM; an order of magnitude higher than that of 15F11³⁵) measured in vitro using surface plasmon resonance (FIG. 13).

Single Molecule Tracking of 1×HA-Tagged Proteins in Living Cells:

Given the high affinity and long binding time of the HA frankenbody to HA epitopes in living cells, in principle the frankenbody can be used to track single HA-tagged proteins. To demonstrate this, histone 1×HA-mCh-H2B proteins were tracked in cells with FB-Halo. To increase the number of tracks, TMR Halo ligand was treated with 50 mM NaBH₄ for 10 minutes before staining cells. This treatment causes the FB-Halo-TMR to spontaneously blink and also respond to 405 nm photoactivation⁴⁵. This allowed for super-resolved, dSTORM-like imaging in living cells, similar to what the Rothbauer lab has done with the BC2 nanobody¹⁵.

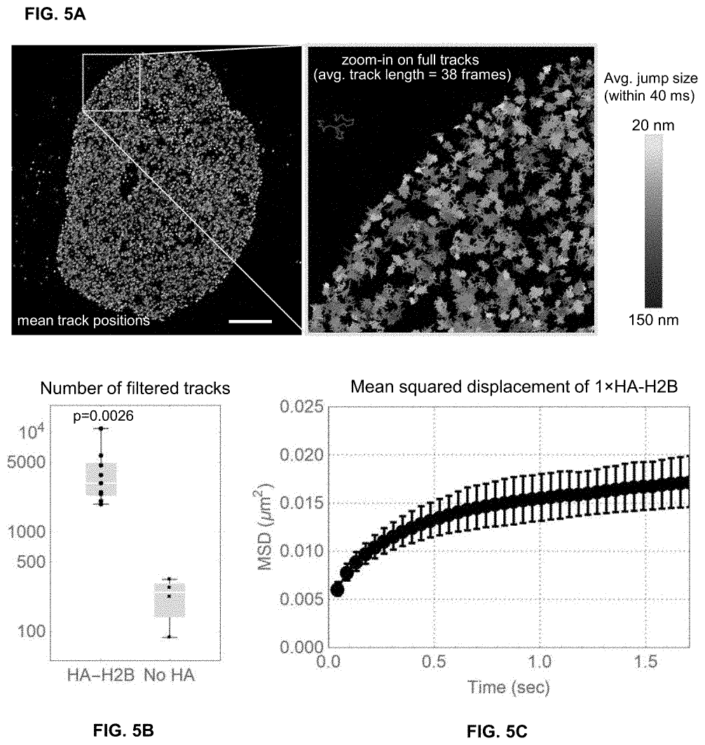

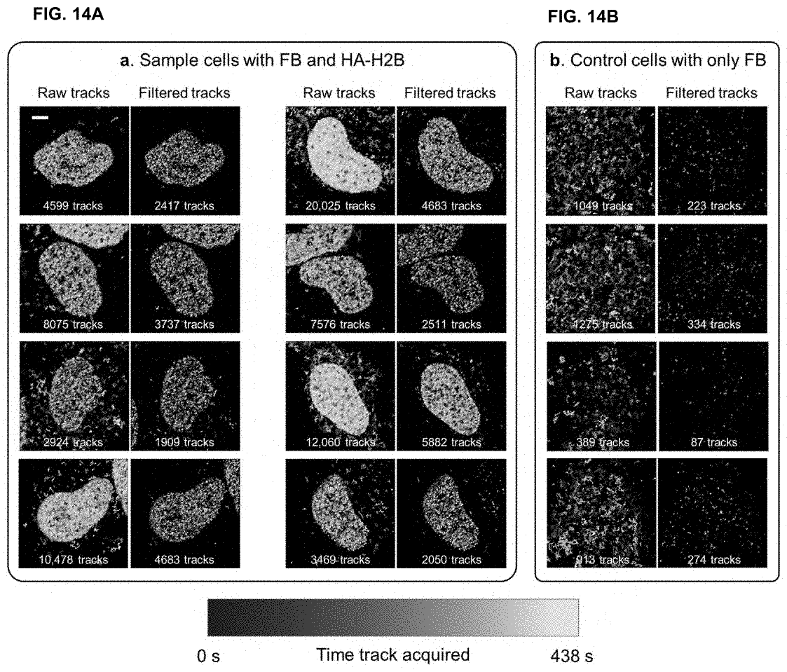

In each experiment a single z-plane was imaged passing through the nucleus at a frame rate of 43.8 ms for a total of 7.3 min (10,000 frames total). At these settings, it was possible to localize on the order of ˜10⁶ FB-Halo per cell, generating around 10³-10⁴ tracks per cell (FIG. 14A). Consistent with the live-cell images of 1×HA-mCh-H2B in FIG. 2A, nearly all FB-Halo tracks were within the nucleus (FIG. 14A), suggesting almost all FB-Halo tracks represent FB-Halo bound to target 1×HA-mCh-H2B. To eliminate the small number of tracks corresponding to unbound FB-Halo, tracks were filtered such that their length had to be at least 16 consecutive frames and jumps between frames had to all be less than 220 nm. This criteria has been used in the past to distinguish chromatin-bound from unbound transcription factors⁴⁶. After filtering, 10³-10⁴ tracks were still left, whereas in control cells lacking 1×HA-mCh-H2B, one or two orders of magnitude fewer tracks were left (and these did not display nuclear localization) (FIG. 5B and FIG. 14B). From the filtered tracks, a map of the mobility of histones across the nucleus was generated (FIG. 5A). This map revealed histones near the nuclear periphery have reduced mobility, consistent with state-of-the-art single-molecule tracking experiments of histone H2B using photoactivatable H2B-PA-mCh⁴⁷. Moreover, the average mean squared displacement of the filtered 1×HA-mCh-H2B tracks displayed similar constrained diffusivity as H2B-PA-mCh⁴⁷ (FIG. 5C). It was therefore concluded that HA frankenbody can be used to faithfully track 1×HA-tagged proteins, provided their mobility and/or localization is distinct from unbound frankenbody.

Tracking Single mRNA Translation in Living U2OS Cells:

A major advantage of the HA frankenbody over other intrabodies is the small size and linearity of its epitope, just 9 aa in length. This means the epitope is quickly translated by the ribosome and becomes available for binding almost immediately. The HA frankenbody therefore has the potential to bind HA-tagged nascent peptides co-translationally, much like purified anti-HA antibody fragments are capable of¹⁷. By simply repeating the HA epitope multiple times within a tag, fluorescence can furthermore be amplified for sensitive single molecule tracking²².

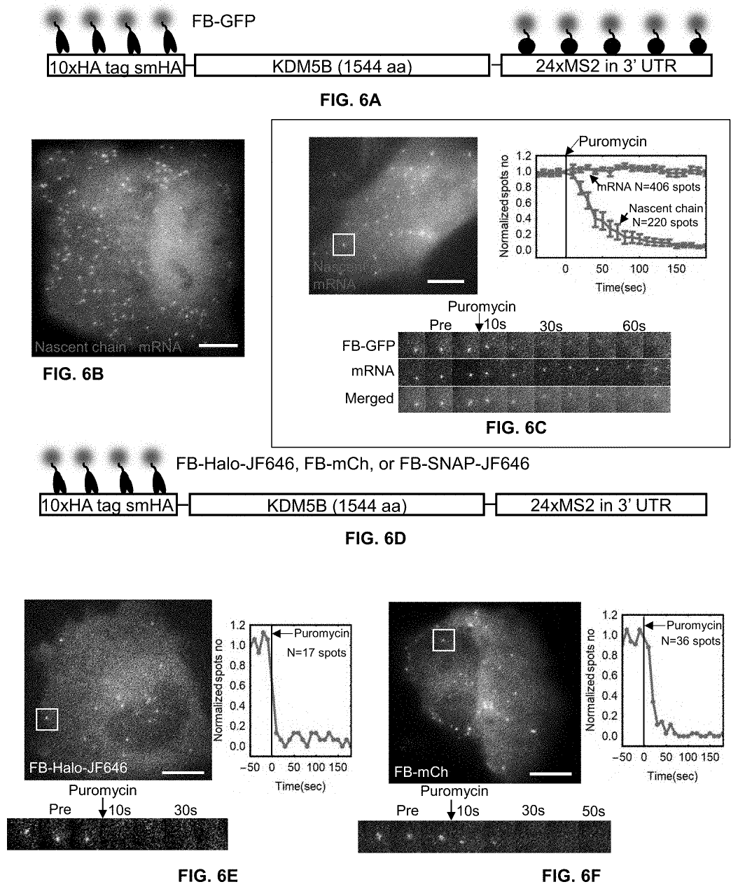

To test the potential of HA frankenbody for imaging translation dynamics, a GFP-tagged version was co-transfected into U2OS cells together with the standard translation reporter. The reporter encodes a 10×HA spaghetti monster tag N-terminally fused to the nuclear protein KDM5B. In addition, the reporter contains a 24×MS2 stem loop repeat in the 3′ UTR to label and track single mRNA (FIG. 6A). A few hours after transfection, single mRNA (labeled by HaloTag-MS2 coat protein, MCP-HaloTag, and the JF646 HaloTag ligand⁴⁸) could be seen diffusing throughout the cell cytoplasm. HA frankenbody co-moved with many of these mRNA (FIG. 6B), indicative of active translation and similar to what is seen with anti-HA antibody fragments¹⁷. To prove these were real translation sites, the translational inhibitor puromycin was added. As expected, this caused the frankenbody to disperse from all single mRNA sites within seconds (FIG. 6C). This confirms the HA frankenbody can indeed bind nascent peptide chains co-translationally.

To ensure the frankenbody can also light up translation sites in multiple colors, experiments were repeated, but now using other frankenbody constructs. For this, cells were co-transfected with mCherry, HaloTag, and SNAP-tag frankenbody plasmids (FB-mCh, FB-Halo, and FB-SNAP), together with a KDM5B translation reporter (FIG. 6D). For mCherry and HaloTag, bright translation sites that responded to puromycin treatment were easily detect (FIG. 6E and FIG. 6F). With SNAP-tag, on the other hand, results were less clear. Although some bright puncta in cells could be seen, not all of these puncta responded to puromycin (suggesting there may be some non-specific aggregation induced by the SNAP-tag). Nevertheless, the data using mGFP-, mCherry-, and HaloTag-frankenbody demonstrate translation can be imaged in at least three colors spanning the imaging spectrum.

Multiplexed Imaging of Single mRNA Translation Dynamics in Living U2Os Cells:

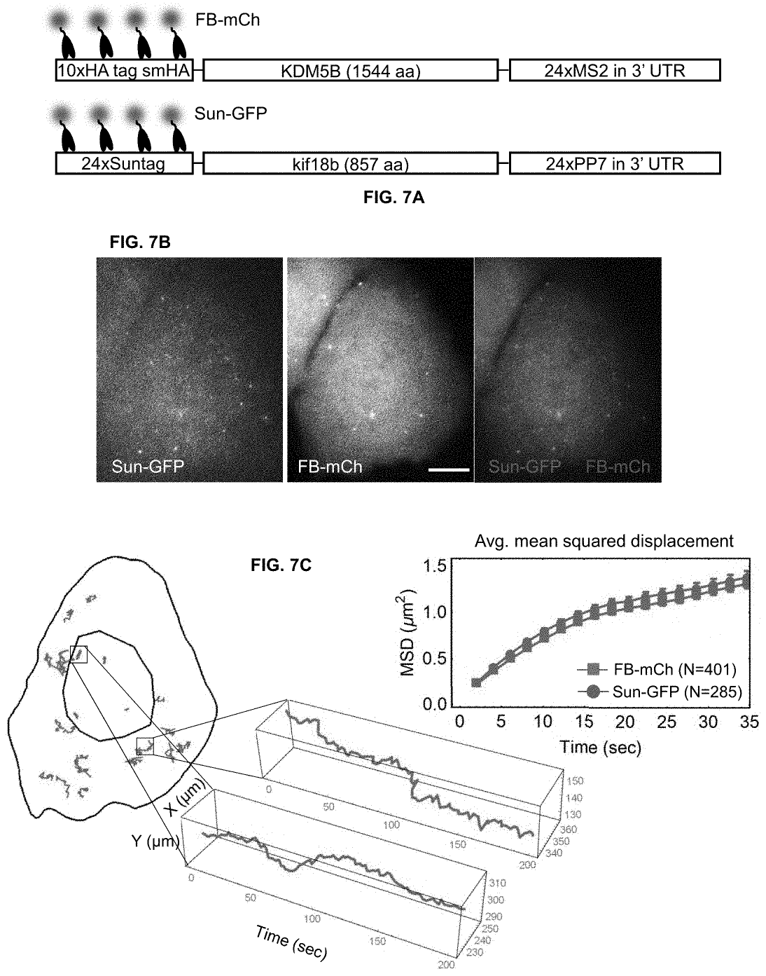

With the ability to image translation in more than one color, the HA frankenbody was combined with the SunTag imaging system to simultaneously quantify the translation kinetics of two distinct mRNA species co-expressed in single living cells. Previously, the SunTag scFv has been fused to GFP (Sun-GFP) to monitor translation¹⁸⁻²¹. This probe²³ (after removing its HA epitope) was then coupled with a complementary mCherry-tagged frankenbody probe (FB-mCh) (FIG. 7A). Co-transfecting these into living U2OS cells together with plasmids encoding SunTag-kif18b and smHA-KDM5B, two distinct types of translation sites were observed, those labeled entirely green by Sun-GFP and those labeled entirely magenta by frankenbody (FIG. 7B). After co-tracking hundreds of these translation sites, their mobilities were quantified. This revealed they both move with a similar kinetic, having a diffusion coefficient (over short timescales) of 0.016±0.004 μm²/sec (95% CI) for FB-mCh and 0.019±0.006 μm²/sec (95% CI) for Sun-GFP. The similarity of their movement despite their different sequences shows that different mRNA types can nonetheless be translated in similar micro-environments.

Since both translation sites were labeled in different colors, the next question was if they ever co-localized. The observation of co-localized translation sites would provide further evidence for multi-RNA “translation factories.” ^{17,21} In particular, it has been shown that approximately 5% of a smHA-KDM5B mRNA reporter is within multi-RNA factories¹⁷. Despite looking, co-localized magenta and green translation sites were not detected. Since the smHA-KDM5B mRNA has different 3′ and 5′ UTRs as well as a different ORF than SunTag-kif18b mRNA, these data suggest that the composition of factories may be dictated in part through mRNA sequence elements.

Monitoring Local Translation in Living Neurons with the HA FrankenbodyI:

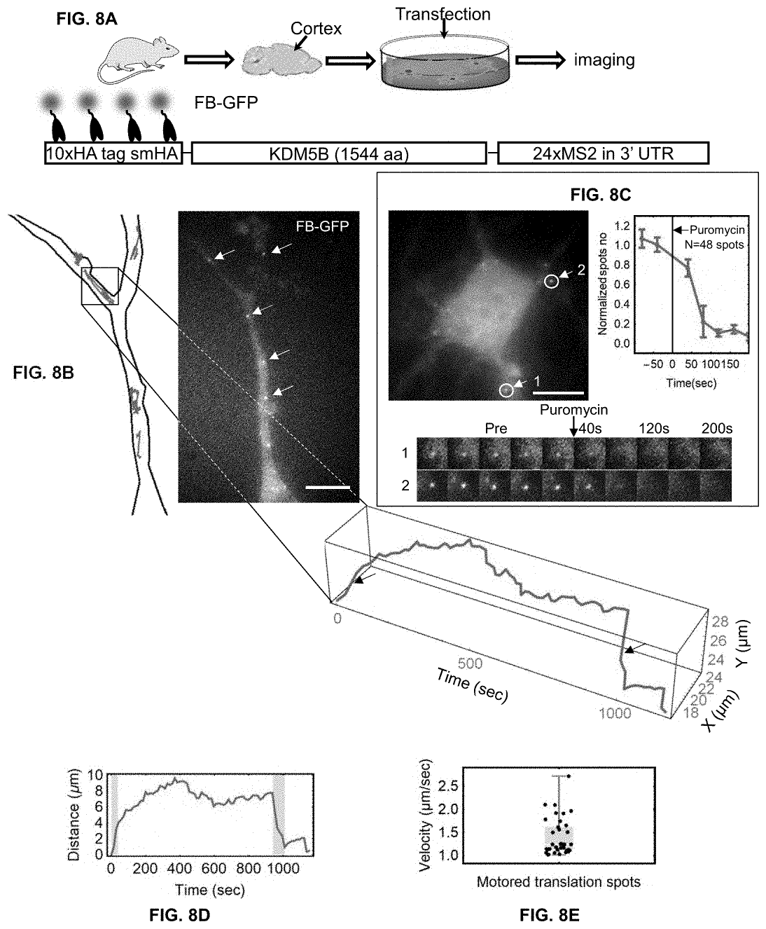

Local translation is implicated in neuronal plasticity, memory formation, and disease⁴⁹. The ability to image local translation at the single molecule level in living neurons would therefore be a valuable research tool to better understand these processes. To facilitate this, the HA frankenbody was tested for its use in monitoring single mRNA translation in living primary rat cortical neurons. When these cells were co-transfected with a KDM5B reporter¹⁷ and the GFP-tagged HA frankenbody (FIG. 8A), distinct bright spots were seen that diffused throughout the cell cytoplasm (FIG. 8B), reminiscent of the translation sites observed in U2OS cells. Again, these were confirmed to indeed be translation sites by adding the translational inhibitor puromycin (FIG. 8C). Just seconds after the drug was added, the bright spots disappeared, just as they had in U2OS cells.

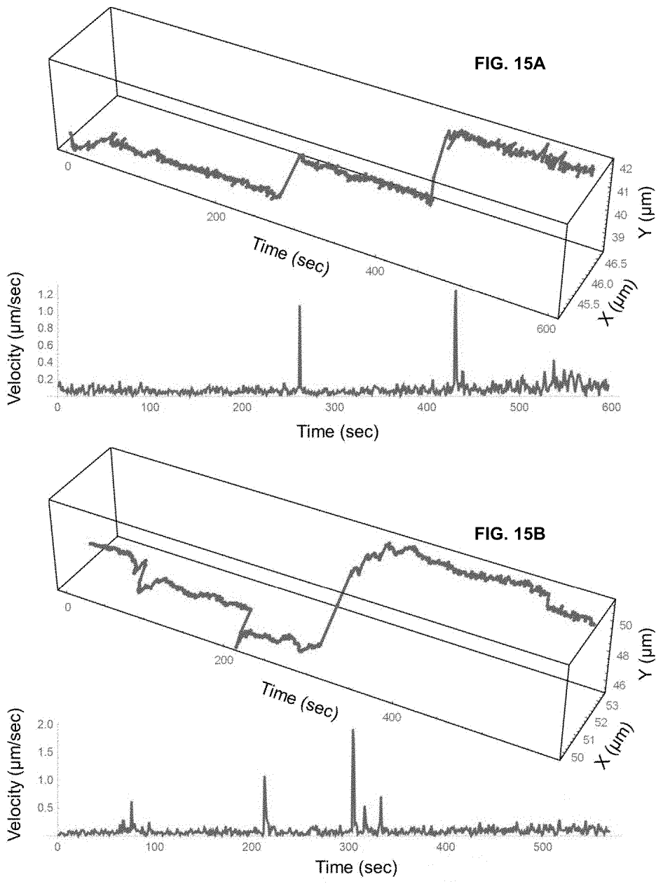

Unlike the mobility of mRNA observed in U2OS cells, mRNA within neuronal dendrites displayed obvious directed motion events. For example, mRNA that zipped along linear paths within dendrites were regularly seen, achieving rapid translocations with rapid retrograde and anterograde transport over large distances up to 8 microns (FIG. 8D, FIG. 8E, and FIG. 15). The strong frankenbody signal within these fast-moving sites suggests translation is still active, despite the motored movement. These data therefore provide further support for a model in which translation is not necessarily repressed during trafficking^{20,21}. Also, since the KDM5B translation reporter is the same as the one used in U2OS cells (FIG. 6 and FIG. 7), these data suggest the cell type and/or local environment plays a significant role in dictating translation site mobility.

Monitoring HA-Tagged Proteins in Zebrafish Embryos with the HA Frankenbody:

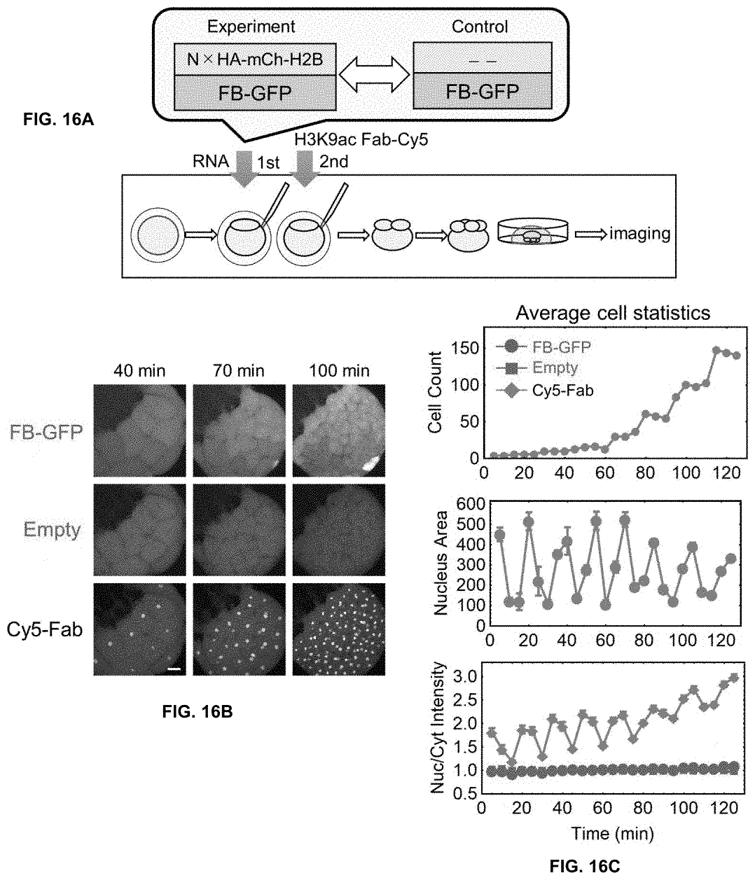

Arguably the most demanding types of imaging applications are in whole living animals. To verify that the HA frankenbody can also be applied in this way, development in zebrafish embryos was monitored. The environment within embryos is complex and contains many potential non-specific binding targets. To express HA frankenbody in this complex environment, mRNA encoding GFP-tagged frankenbody and HA-mCh-H2B was microinjected into the yolk of one-cell stage zebrafish eggs. With this setup, HA frankenbody is expressed (i.e. translated) immediately without having to wait for the onset of transcription after the maternal-zygotic transition⁵⁰. Following the initial injection, a positive control Fab (Cy5 conjugated), which specifically binds and lights up endogenous histone acetylation (H3K9ac) in the cell nucleus²⁵ (FIG. 16A), was co-loaded.

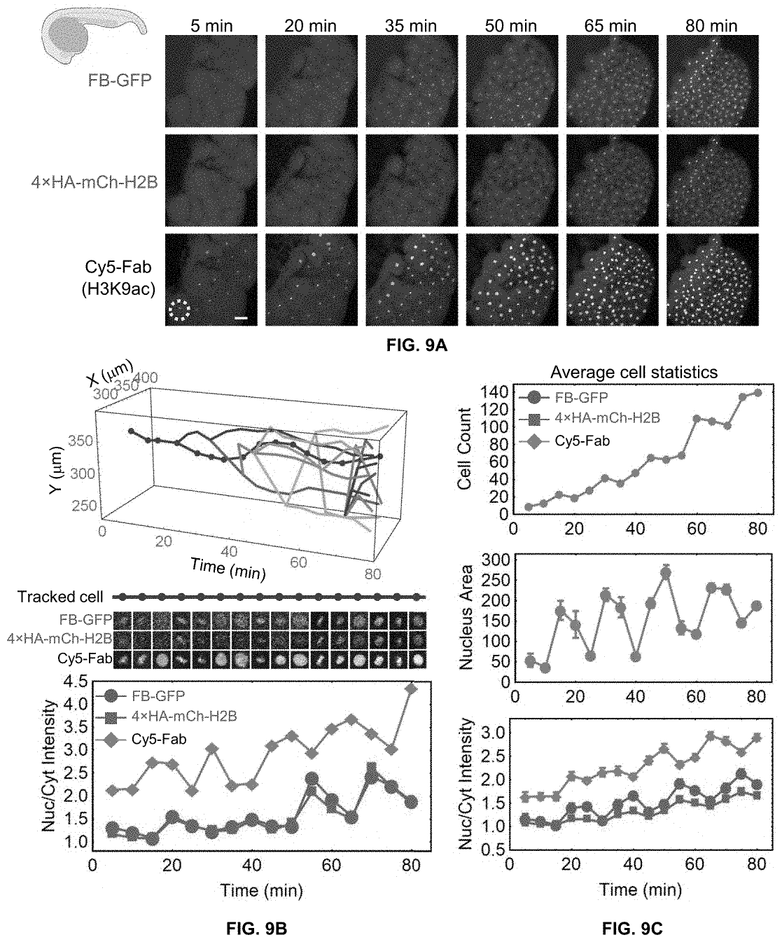

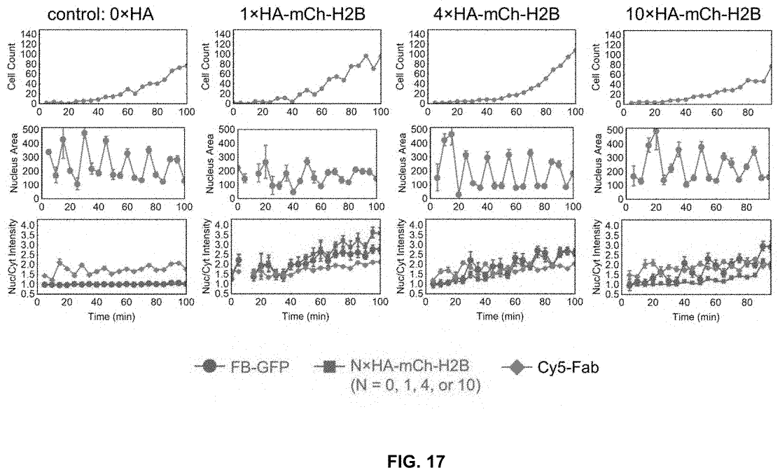

Embryo development was first imaged with the HA frankenbody around the four- or eight-cell stage. At all timepoints colocalization of the frankenbody with the 4×HA-mCh-H2B target could be seen (FIG. 9A), although at earlier timepoints the concentrations of both were lower and therefore marked the nuclei only dimly compared to the positive control Fab. Nevertheless, all three signals were detected in the nuclei of single mother and daughter cells throughout the entire 80-minute imaging time course (FIG. 9B, upper). Moreover, the signal from the frankenbody in the nucleus tightly correlated with the target 4×HA-mCh-H2B signal, increasing steadily from a nuclear-to-cytoplasmic ratio of one to nearly 2.5 (FIG. 9B, lower). This single-cell trend was also observed in the population of cells (FIG. 9C). As a negative control, experiments were repeated in zebrafish embryos lacking target HA-mCh-H2B. In this case, the frankenbody was evenly distributed throughout the embryo (FIG. 16B, FIG. 16C), displaying a nuclear-to-cytoplasmic ratio close to one at all times. To see how signal varies with the number of HA epitopes, experiments were repeated with N×HA-mCh-H2B (N=1,4,10; FIG. 17). In all cases, cell nuclei were tracked with HA frankenbody but signal improved as more epitopes were added to the tag. In fact, for N=10, the HA frankenbody nuclear-to-cytoplasm ratio was higher than mCherry for almost all timepoints (FIG. S7). This confirms the HA frankenbody binds the HA epitope selectively and tightly in vivo, so it can be used to accurately monitor the concentration of target HA-tagged proteins in living organisms.

Discussion:

While scFvs have great potential for live-cell imaging, so far there are just a few documented examples of scFvs that fold and function in the reducing environment of living cells. Here CDR loop grafting was employed to generate stable and functional scFvs that bind the classic linear HA epitope tag in vivo. The resulting HA frankenbody is capable of labeling HA-tagged nuclear, cytoplasmic, membrane, and mitochondrial proteins in multiple colors and in a diverse range of cellular environments, including primary rat cortical neurons and zebrafish embryos.

A major advantage of the HA frankenbody is it can be used to image single mRNA translation dynamics in living cells. This is because the target HA epitope (SEQ ID NO: 23) is small (9 a.a.) and linear. It therefore emerges quickly from the ribosome, so it can be co-translationally labeled by frankenbody almost immediately. It is also short enough to repeat many times in a single tag for signal amplification, as in the HA spaghetti monster tag²². In principle, epitopes could even be made conditionally accessible within a protein to monitor conformational changes. In contrast, almost all other antibody-based probes bind to 3D epitopes that span a large length of linear sequence space. In general, 3D epitopes take a relatively long time to be translated and fold before they become accessible for probe binding. Furthermore, they are too big to repeat more than a few times, so fluorescence is difficult to amplify to the degree necessary for single molecule tracking^{22,23}.

Here the HA frankenbody was used to image single mRNA translation in both living U2OS cells and primary neurons. Unlike Fab, which cause neurons to peel during the loading procedure, HA frankenbody can be expressed in neurons without issue via transfection. This was exploited to demonstrate the mobility of translating mRNA is cell-type dependent. While in these experiments the KDM5B translation reporter mRNA displayed largely non-directional, diffusive movement in U2OS cells, in neurons they were often motored. Neurons can be notoriously long, so motored mRNA movement provides a solution to the unique challenge of local protein production in distal neuronal dendrites and axons^{51,52}. An open question is if translation is repressed during transport. On the one hand, certain mRNA are known to be actively repressed during trafficking^{3,53}, perhaps to conserve energy. On the other hand, the Singer and Bertrand labs have recently shown that mRNA can be actively translated during transport^{20,21}. Here a similar phenomenon is reported, with the KDM5B translation reporter being rapidly motored over distances up to 8 microns in dendrites while retaining a strong translation signal. As the KDM5B reporter encodes the β-actin 3′ and 5′ UTR, the data demonstrate that the motored transport of translating β-actin transcripts within neurons is controlled by the 3′ or 5′ UTRs rather than the ORF.

Besides the HA frankenbody, there is only one other scFv capable of imaging single mRNA translation dynamics: the SunTag scFv^{18,20,21,30,31}. Compared to the SunTag, the HA frankenbody binds it target epitope with lower affinity (nM versus pM). However, the SunTag epitope (SEQ ID NO: 24) is over twice the length of the HA epitope²³. Furthermore, the relatively new SunTag is not as ubiquitous as the HA-tag, which has enjoyed widespread use in the biomedical sciences for over thirty years³⁵.

While the HA frankenbody and SunTag each have unique advantages, their combination creates a powerful genetically-encoded toolset to quantify single mRNA translation dynamics in two complementary colors. For example, their combination makes it possible to combine HA- and SunTag-epitopes in single mRNA reporters and thereby examine more than one open reading frame at a time or to create a gradient of translation colors for multiplexed imaging. In this study, the HA frankenbody with the SunTag were combined to quantify the spatiotemporal dynamics of two distinct mRNA species with different UTRs and ORFs. Unlike earlier work with two mRNA species sharing common UTRs, in this case colocalization of translation sites, i.e. “translation factories,” was not observed. This suggests that specific sequences within mRNAs likely dictate which translation factories they are recruited to, if any.

The genetic encodability of the HA frankenbody makes it a great value to researchers who have so far had to rely on expensive full-length antibodies purified from hybridoma cells to label HA-tagged proteins of interest. The HA frankenbody will therefore have an immediate and positive impact on the large cadre of researchers already employing the HA-tag in their studies, both in vitro and in vivo. With the HA frankenbody, researchers can simply transfect cells or animals expressing HA-tagged proteins with DNA or mRNA encoding the frankenbody fused to a fluorescent protein. This enables the visualization and quantification of HA-tagged protein expression, localization, and dynamics in living systems, both at the single molecule, as in live-cell dSTORM imaging experiments, as well as across entire organisms, as in zebrafish embryo experiments. While 1×HA tags are adequate in both cases, it was demonstrated that fluorescence signal in zebrafish embryos can be significantly amplified by simply repeating HA epitopes within tags (FIG. 9B, FIG. 9C, and FIG. 16). This will be useful for the rapid and sensitive detection of short-lived HA-tagged proteins in vivo, similar to how the GFP-nanobody has been used to both amplify GFP fluorescence^{13,14} and image the spatiotemporal dynamics of short-lived, GFP-tagged transcription factors during Drosophila development⁵. Finally, the frankenbody can be genetically fused to other protein motifs to create a wide array of tools for live-cell imaging or manipulation of HA-tagged proteins, or it can be co-evolved with the HA-epitope into other complementary probe/epitope pairs via directed evolution techniques^{54,55}. As shown in FIG. 10, the sequences of the two successful HA-scFv variants (χ_{2E2}^{HA} and χ_{15F11}^{HA}) are slightly different, but their performance in living cells is almost identical according to the initial screen. Thus, many positions of the frankenbody can be mutated without destroying its functionality, demonstrating great potential for further evolution. It is therefore anticipate the HA frankenbody will be a useful new imaging reagent to complement the growing arsenal of live-cell antibody-based probes^{23,56-58}.

Given the success with CDR grafting in this study, in principle it should be relatively straightforward to generate more scFvs that bind additional targets besides the HA epitope tag. However, the functionality of CDR grafted scFvs is still difficult to predict³³, so it remains unclear how generalizable the method is. According to the initial screen of scaffolds for frankenbodies, scFvs that have similar sequences will generally have a higher chance of being compatible grafting partners. We are therefore optimistic that as the price of determining antibody sequences continues to decrease, more scFvs will be constructed, tested, and verified to fold and function in living cells. The availability of compatible grafting partners will therefore only increase, meaning less effort will be required to generate additional frankenbodies in the future. Besides scFv, another promising option is the continued development of nanobodies. For example, we recently were made aware of a nanobody capable of binding a repeated 15 aa epitope co-transaltionally⁵⁹ (coined the “MoonTag”). Ultimately, as more and more genetically-encoded epitope binders become available, we envision a panel of such probes will allow researchers to better capture the full lifecycles of proteins in vivo with high spatiotemporal resolution.

Methods—Plasmid Construction:

Each chimeric anti-HA scFv tested in this study was constructed by grafting six CDR loops of an anti-HA antibody 12CA5 onto each selected scFv scaffold. The χ_{15F11}^{HA} plasmid was constructed in two steps: 1) a CDR-loop grafted scFv gblock was synthesized in vitro and ligated to a H4K20me1 mintbody 15F11 vector³⁸ cut by EcoRI restriction sites via Gibson assembly (House prepared master mix); 2) the linker connecting scFv and EGFP, as well as EGFP, was replaced by a flexible (G₄S)×5 linker and the monomeric EGFP by Gibson assembly through NotI restriction sites. For the other 4 chimeric scFv plasmids, each CDR-loop grafted scFv gblock was synthesized in vitro and ligated into the χ_{15F11}^{HA} vector cut by EcoRI restriction sites via Gibson assembly.

The target plasmid 1×HA-mCh-H2B was constructed by replacing the sfGFP of an Addgene plasmid sfGFP-H2B (Plasmid #56367) with a 1×HA epitope tagged mCherry gblock (1×HA-mCh) synthesized in vitro, in which a NotI restriction site was inserted between the 1×HA epitope and the mCherry coding sequence. The 4×HA-mCh-H2B was constructed by replacing the 1×HA tag of the 1×HA-mCh-H2B construct with a 4×HA-epitope gblock (4×HA) synthesized in vitro through AgeI and NotI restriction sites. The 10×HA-mCh-H2B (smHA-mCh-H2B) was constructed by replacing the 1×HA tag of the 1×HA-mCh-H2B construct with a 10×HA spaghetti monster (smHA) amplified from an Addgene plasmid smHA-KDM5B (Addgene plasmid #81085) using primers NZ-098 and 099 (All primer sequences are shown in Supplementary Table 1). The smHA-H2B was constructed by replacing the 1×HA-mCh of the 1×HA-mCh-H2B construct with smHA amplified from the smHA-KDM5B (Addgene plasmid #81085) using primers NZ-098 and 100.

Another target plasmid 4×HA-mCh-β-actin was constructed by replacing the H2B of the 4×HA-mCh-H2B with a β-actin encoding sequence via BgIII and BamHI restriction sites with a β-actin amplicon. The β-actin was amplified from a published plasmid SM-β-actin¹⁷ using primers NZ-073 and NZ-074.

The 4×HA-mRuby-Kv2.1 plasmid was generated by first amplifying the 4×HA tag from 4×HA-mCh-H2B using the primers NZ-105 and 106. Then the 4×HA amplicon was introduced into a published plasmid pCMV-mRuby2-Kv2.1⁶⁰ (a gift from Dr. Michael Tamkun), which had been linearized by a restriction digest with AgeI, using Gibson assembly. The smHA-Kv2.1 plasmid was generated by PCR amplification of the rat Kv2.1 coding sequence from pBK-Kv2.1⁴⁰ and subsequent ligation into AsiSI and PmeI restriction sites on an Addgene plasmid smHA-KDM5B (Addgene plasmid #81085) using primers Kv2.1-1 and 2.

The H2B-mCh-1×HA was constructed by 2 steps: (1) Replace the 1×HA tag of the 1×HA-mCh-H2B with H2B through AgeI and NotI sites, in which the H2B was amplified from 1×HA-mCh-H2B using the primers NZ-092 and 093; (2) Replace the H2B at the C-terminus of mCherry with a 1×HA tag through BgIII and BamHI sites, in which the 1×HA tag was synthesized by overlapping PCR with the following primers NZ-094 and 095. The Mito-mCh-1×HA was constructed by replacing the H2B in H2B-mCh-1×HA with Mito gblock²³ synthesized in vitro through AgeI and NotI sites. The Mito-mCh-smHA was constructed by replacing the 1×HA tag of Mito-mCh-1×HA with smHA amplified from the smHA-KDM5B using primers NZ-096 and 097. The mCh-H2B was generated by replacing sfGFP of the Addgene plasmid (Plasmid #56367) with an mCherry gblock synthesized in vitro.

The plasmids FB-mCh, FB-Halo, FB-SNAP were built by replacing the mEGFP of χ_{15F11}^{HA} with mCherry, HaloTag and SNAP-tag gblocks synthesized in vitro respectively.

pET23b-FB-GFP, the plasmid for recombinant FB expression and purification, was generated by assembling a FB-GFP gene with a previously built plasmid pET23b-Sso7d^{61,62} by NdeI and NotI restriction sites. The FB-GFP encoding sequence was amplified from χ_{15F11}^{HA} by PCR with primers NZ-075 and 077.

For the translation assay, the reporter construct smHA-KDM5B-MS2 is an Addgene plasmid (Plasmid #81085). The SunTag-kif18b reporter plasmid was purchased from Addgene (Plasmid #74928), and its scFv plasmid (Plasmid #60907) was modified by removing the HA epitope encoded in the linker. The HA epitope was removed by site-directed mutagenesis with QuikChange Lightning (Agilent Technologies) per the manufacturer's instruction using primers HAout-1 and 2.

The gblocks were synthesized by Integrated DNA Technologies and the recombinant plasmids were sequence verified by Quintara Biosciences. The sequences of primers are shown in Supplementary Table 1. All plasmids used for imaging translation were prepared by NucleoBond Xtra Midi EF kit (Macherey-Nagel) with a final concentration about 1 mg mL⁻¹.

Methods—U2OS Cell Culture, Transfection and Bead Loading:

U2OS cells (ATCC HTB-96) were grown in an incubator at 37° C., humidified, with 5% CO₂ in DMEM medium (Thermo Scientific) supplemented with 10% (v/v) fetal bovine serum (Altas Biologicals), 1 mM L-glutamine (Gibco) and 1% (v/v) penicillin-streptomycin (Gibco or Invitrogen).

For tracking protein localization, cells were plated into a 35 mm MatTek chamber (MatTek) 2 days before imaging and were transiently transfected with Lipofectamine™ LTX reagent with PLUS reagent (Invitrogen) according to the manufacturer's instruction 18˜24 hours prior to imaging.

For imaging translation with mRNA labeled by MCP-Halo protein, cells were plated into a 35 mm MatTek chamber (MatTek) the day before imaging. On the day of imaging, before bead loading, the medium in the MatTek chamber was changed to Opti-MEM (Thermo Scientific) with 10% fetal bovine serum. A mixture of plasmids (smHA-KDM5B/FB) and purified MCP-HaloTag protein¹⁷ were bead loaded as previously described^{6,17,26}. Briefly, after removing the Opti-medium from the MatTek chamber, 4 uL of a mixture of plasmids (1 ug each) and MCP-HaloTag (130 ng) in PBS was pipetted on top of the cells and ˜106 μm glass beads (Sigma Aldrich) were evenly distribute on top. The chamber was then tapped firmly 7 times, and Opti-medium was added back to the cells. 3 hours post bead loading, the cells were stained in 1 mL of 0.2 nM of JF646-HaloTag ligand⁴⁸ diluted in phenol-red-free complete DMEM. After a 20 min incubation, the cells were washed three times in phenol-red-free complete DMEM to remove glass beads and unliganded dyes. The cells were then ready for imaging.

For imaging translation without mRNA labeling, the cells plated on MatTek chamber were transiently transfected with the plasmids needed, smHA-KDM5B/FB with or without SunTag-kif18b/Sun (with the HA epitope removed), using Lipofectamine™ LTX reagent with the PLUS reagent (Invitrogen) according to the manufacturer's instruction on the day of imaging. 3 hours post transfection, the medium was changed to phenol-red-free complete DMEM. The cells were then ready for imaging.

Methods—Purification of FB-GFP:

E. coli BL21 (DE3) pLysS cells transformed with pET23b-FB-GFP were grown at 37° C. to a density of OD600 0.6 in 2×YT medium containing Ampicillin (100 mg L⁻¹) and Chloramphenicol (25 mg L⁻¹) with shaking. Isopropyl-β-D-thiogalactoside (IPTG) was added to induce protein expression at a final concentration of 0.4 mM, and the temperature was lowered to 18° C. Cells were harvested after 16 hours by centrifugation and resuspended in PBS buffer supplemented with 300 mM NaCl, protease inhibitors (ThermoFisher), 0.2 mM AEBSF (20 mL L⁻¹ culture) and lysed by sonication. Lysate was clarified by centrifugation. The supernatant was loaded onto 2 connected HisTrap HP 5 ml columns (GE Healthcare), washed and eluted by a linear gradient of 0-500 mM imidazole. The fractions containing the protein of interest were pooled, concentrated using Amicon Ultra-15 30 kDa MWCO centrifugal filter unit (EMD Millipore) and loaded onto a size-exclusion HiLoad Superdex 200 PG column (GE healthcare) in HEPES-based buffer (25 mM HEPES pH 7.9, 12.5 mM MgCl₂, 100 mM KCl, 0.1 mM EDTA, 0.01% NP40, 10% glycerol and 1 mM DTT). The fractions containing FB-GFP protein were collected, concentrated, and stored at −80° C. after flash freezing by liquid nitrogen.

Methods—Immunostaining:

U2OS cells were transiently transfected with 4×HA-mCh-H2B or 4×HA-mCh-β-actin with Lipofectamine™ LTX reagent with the PLUS reagent (Invitrogen). 26 hours post transfection, the cells were fixed with 4% paraformaldehyde (Electron Microscopy Sciences) for 10 minutes at room temperature, permeabilized in 1% Triton 100 in PBS, pH 7.4 for 20 minutes, blocked in Blocking One-P (Nacalai Tesque) for 20 minutes, and stained at 4° C. overnight with purified FB-GFP protein (0.5 ug mL⁻¹ in 10% blocking buffer). The next morning, the cells were washed with PBS, and the protein of interest was imaged by an Olympus IX81 spinning disk confocal (CSU22 head) microscope using a 100× oil immersion objective (NA 1.40) under the following conditions: 488 nm (0.77 mW; measured at the back focal plane of the objective; herein all laser power measurements correspond to the back focal plane of the objective) and 561 nm (0.42 mW) sequential imaging for 50-timepoints without delay, 2×2 spin rate, 100 ms exposure time. Images were acquired with a Photometrics Cascade II CCD camera using SlideBook software (Intelligent Imaging Innovations). The immunostaining images were generated by averaging 50-timepoint images for each channel by Fiji⁶³.

Methods—Western Blots:

U2OS cells were transiently transfected with 4×HA-mCh-H2B or 4×HA-mCh-β-actin with Lipofectamine™ LTX reagent with the PLUS reagent. 10 hours post transfection, the cells were harvested and lysed in 120 μL of RIPA buffer with cOmplete Protease Inhibitor (Roche). 6.5 μL of each cell lysate was loaded on a NuPAGE™ 4%˜12% Bis-Tris protein gel (Invitrogen) and run for 60 minutes at 100 V and 25 minutes at 200 V. Proteins were transferred to a PVDF membrane (Invitrogen), blocked in blocking buffer (5% milk powder in 0.05% TBS-Tween 20) for 1 hour, and stained overnight with either purified FB-GFP protein (0.5 ug mL⁻¹ in blocking buffer) or anti-HA parental antibody 12CA5 (Sigma-Aldrich Cat #11583816001; 2000-fold dilution with final concentration 0.5 ug mL⁻¹ in blocking buffer). For the primary antibody 12CA5, an additional incubation for 1 hour with anti-mouse antibody/Alexa488 (Thermo Fisher Cat # A11001; 5000-fold dilution in blocking buffer) was done. The protein of interest was detected from the GFP fluorescence for FB-GFP or Alexa 488 for anti-HA antibody using a Typhoon FLA 9500 (GE Healthcare Life Sciences) with the following conditions: excitation wavelength 473 nm, LPB filter (≥510 nm), 300 V photomultiplier tube and 10 μm pixel size. The uncropped and unprocessed western blots are supplied in FIG. 12.

Methods—Fluorescence Recovery after Photobleaching (FRAP):

To study the binding affinity of FB to HA epitopes in living cells, FRAP experiments were performed on cells transiently transfected with 4×HA-mCh-H2B (1.25 ug) and FB-GFP (1.25 ug) 24 hours before FRAP. The images were acquired using an Olympus IX81 spinning disk confocal (CSU22 head) microscope coupled to a Phasor photomanipulation unit (Intelligent Imaging Innovations) with a 100× oil immersion objective (NA 1.40). Before photobleaching, 20 or 10 frames were acquired with 1 sec or 5 sec time interval. The images were captured using a 488 nm laser (0.77 mW) laser with 100 msec exposure time followed by 561 nm (0.42 mW) laser with 15 msec exposure time. The spinning disk was set up at 1×1 spin rate. After acquiring pre-FRAP images, the p488 nm laser (from the Phasor unit for photobleaching) at 17 mW with 100 msec exposure time, or 4 mW with 500 msec exposure, was used to photobleach a circular region in the nucleus. After photobleaching, 30 images were captured without a time interval delay or with 500 msec delay, and then 100 images with a 1 sec time interval delay and 100 images with a 5 sec time interval delay were acquired using the same imaging settings as the pre-FRAP images. The fluorescent intensity through time of the photobleached spot were exported using the Slidebook software. The fluorescent intensity of the nucleus and background were obtained by Fiji⁶³ after correcting for cell movement using the StackReg Fiji plugin⁶⁴. The FRAP curve and t_{half} were obtained using easyFRAP-web⁶⁵, according to the website instructions. FIG. 4B and FIG. 4D were generated by Mathematica (Wolfram Research).

Methods—MCP Purification:

MCP-HaloTag was purified by immobilized metal affinity chromatography¹⁷. Briefly, the His-tagged MCP-HaloTag was purified through a Ni-NTA-agarose (Qiagen) packed column per the manufacturer's instructions, with minor modifications. E. coli expressing the interested protein was lysed in a PBS buffer with a complete set of protease inhibitors (Roche) and 10 mM imidazole. The resin was washed with PBS-based buffer containing 20 and 50 mM imidazole. The protein was then eluted in a PBS buffer with 300 mM imidazole. The eluted His-tagged MCP was dialyzed in a HEPES-based buffer (10% glycerol, 25 mM HEPES pH 7.9, 12.5 mM MgCl₂, 100 mM KCl, 0.1 mM EDTA, 0.01% NP-40 detergent, and 1 mM DTT), snap-frozen in liquid nitrogen, and then stored at −80° C.

Methods—Nascent Chain Tracking:

The reporter plasmid smHA-KDM5B and the FB construct were either transiently transfected without the MCP-HaloTag protein or bead loaded with MCP-HaloTag protein into U2OS cells plated on a 35 mm MatTek chambers 4˜6 hours before imaging. 3 hours later, if MCP-HaloTag protein was bead loaded, the cells were stained with the JF646-HaloTag ligand, then washed with phenol-red-free complete DMEM medium. If no MCP-HaloTag protein was needed, the medium of the cells was changed to phenol-red-free complete DMEM medium 3 hours post transfection. The cells were then ready for imaging.

For multiplexed imaging, 2 reporter plasmids, smHA-KDM5B and SunTag-kif18b, as well as 2 probes, FB-mCh and Sun-GFP (with the HA epitope removed), were transiently transfected into U2OS cells plated on a MatTek chamber 4˜6 hours before imaging. 3 hours later, the medium of the cells was changed to phenol-red-free complete DMEM medium. The cells were then ready for imaging.

Methods Imaging—Condition for Translation and Colocalization Assays:

To image single mRNAs and their translation status with FB, a custom-built widefield fluorescence microscope based on an inclined illumination (HILO) scheme was used^{17,66}. Briefly, the excitation beams, 488, 561, 637 nm solid-state lasers (Vortran), were coupled and focused off-axis on the rear focal plane of the objective lens (APON 60×OTIRF, Olympus). All reported laser powers were measured at the back-focal plane of the objective. The emission signals were split by an imaging grade, ultra-flat dichroic mirror (T660Ipxr, Chroma). The longer emission signals (far-red) after splitting were passed through a bandpass filter (FF01-731/137-25, Semrock). The shorter emission signals (red and green) after splitting were passed through either a bandpass filter for red (FF01-593/46-25, Semrock) or a bandpass filter for green (FF01-510/42-25, Semrock) installed in a filter wheel (HS-625 HSFW TTL, Finger Lakes Instrumentation). The longer (far-red) and the shorter (red and green) emission signals were detected by separate two EM-CCD cameras (iXon Ultra 888, Andor) by focusing with a 300 mm achromatic doublet lenses (AC254-300-A-ML, Thorlabs). The combination of 60× objective lens from Olympus, 300 mm tube lens, and iXon Ultra 888 produces 100× images with 130 nm pixel⁻¹. A stage top incubator for temperature (37° C.), humidity, and 5% CO₂ (Okolab) is equipped on a piezoelectric stage (PZU-2150, Applied Scientific Instrumentation) for live cell imaging. The lasers, the cameras, the piezoelectric stage, and the filter wheel were synchronized by an open source micro controller, Arduino Mega board (Arduino). Imaging acquisition was performed using open source Micro-Manager software (1.4.22)⁶⁷.

The imaging size was set to the center 512×512 pixels² (66.6×66.6 μm²), and the camera integration time was set to 53.64 msec. The readout time of the cameras from the combination of our imaging size, readout mode (30 MHz), and vertical shift speed (1.13 μsec) was 23.36 msec, resulting in our imaging rate of 13 Hz (70 msec per image). Red and green signals were imaged alternatively. The emission filter position was changed during the camera readout time. To minimize the bleed-through, the far-red signal was simultaneously imaged with the green signal. To capture the whole thickness of cells, 13 z-stacks with a step size of 500 nm (6 μm in total) were imaged using the piezoelectric stage. This resulted in our total cellular imaging rate of 1 Hz for imaging either red or green signals, and 0.5 Hz for imaging both red and green signals regardless of far-red imaging.

For FIG. 1C, FIG. 1D, FIG. 2A, and FIG. 2E, a single plane of the cells was imaged continuously at 6.5 Hz for 100 time points and averaged throughout the time (Lasers: 488 nm, 130 μW; 561 nm, 4 μW (FIG. 1C), 90 μW (FIG. 1D), 19 μW (FIG. 2A), 155 μW (FIG. 2E); 637 nm (220 μW)). For FIG. 2B (left), the cell was imaged continuously at 0.5 Hz, 488 nm (100 μW) and 561 nm (10 μW) lasers with 13 z-stacks every timepoint and averaged throughout the time. The acquired averaged 13 z-stacks were deconvolved using Fiji. For FIG. 6B, FIG. 6C, FIG. 6E, and FIG. 6F, cells were imaged every 10 sec with 13 z-stacks per timepoint (Lasers: 488 nm, 13 μW (FIG. 6B, 18 μW (FIG. 6C); 561 nm, 172 μW (FIG. 6F); 637 nm, 150 μW (FIG. 6B and FIG. 6C), 35 μW (FIG. 6E)). For FIG. 7B, the cell was imaged continuously at 0.5 Hz with 13 z-stacks every timepoint (Lasers: 488 nm, 130 μW; 561 nm, 172 μW). For FIG. 8B, the cells were imaged every 14 sec with 13 z-stacks every timepoint (Laser: 488 nm, 70 μW). For FIG. 8C, cells were imaged every 40 sec with 13 z-stacks every timepoint (Laser: 488 nm, 130 μW). For Motored translation spots velocity determination (FIG. 8E), the neurons were imaged continuously at 1 Hz with 13 z-stacks every timepoint.

For FIG. 2B (right) and FIG. 2C, the co-localization was imaged by the Olympus IX81 spinning disk confocal (CSU22 head) microscope described before using a 100× oil immersion objective (NA 1.40) under the following conditions: 488 nm (0.77 mW) and 561 nm (0.42 mW) sequential imaging for 5 timepoints without delay with multiple z slices to cover the whole cell body for each time point, 1×1 spin rate, exposure time adjusted by cell brightness. Images were acquired with a Photometrics Cascade II CCD camera using SlideBook software (Intelligent Imaging Innovations). The displayed images in figures were generated by averaging 5 timepoints and then a max-projection of all z-slices was performed by Fiji⁶³.

Methods—Particle Tracking of Translation Sites:

Single translation site detection and tracking was performed on maximum intensity projection images with custom Mathematica (Wolfram Research) code¹⁷. Briefly, the images were processed with a bandpass filter to highlight particles, and then binarized to detect their intensity-centroids as positions using the built-in Mathematica routine ComponentMeasurements. Detected particles were tracked and linked through time via a nearest neighbor search. The precise coordinates (super-resolved locations) of mRNAs and translation sites were determined by fitting (using the built-in Mathematica routine NonlinearModelFit) the original images to 2D Gaussians of the following form:

$$I ( x , y ) = {I}_{BG} + {Ie}^{- \frac{{( x - {x}_{0} )}^{2}}{2 {σ}_{x}^{2}} - \frac{{( y - {y}_{0} )}^{2}}{2 {σ}_{y}^{2}}} \qquad ( 1 )$$

where I_{BG} is the background fluorescence, I the particle intensity, (x₀, y₀) the particle location, and (σₓ, σ_{y}) spreads of the particle. The offset between the two cameras was registered using the built-in Mathematica routine FindGeometricTransform to find the transform function that best aligned the fitted positions of 100 nm diameter Tetraspeck beads evenly spread out across the image field-of-view.

For FIG. 6C, FIG. 6E, FIG. 6F, FIG. 7C, FIG. 8B and FIG. 8D, particles were tracked by custom Mathematica code and further plotted with Mathematica. For FIG. 7C, average mean squared displacements were calculated from the Gaussian-fitted coordinates (from 2D maximum intensity projection images). The diffusion constant was obtained by fitting the first 5 time points to a line with slope m=4D, where D is the diffusion coefficient. The single motored translation spots (FIG. 8E) were tracked by the Fiji plugin TrackMate⁶⁸ after max-projection and further plotted with Mathematica. All Mathematica code is available upon request.

Methods—Single Molecule Tracking of 1×HA-Tagged Proteins in Living Cells:

The day before imaging, cells were plated on MatTek chamber and transiently transfected with the 1×HA-mCh-H2B and FB-Halo using Lipofectamine™ LTX reagent with the PLUS reagent (Invitrogen) according to the manufacturer's instruction. 3 hours post transfection, the medium was changed to complete DMEM. Before imaging, the cells were stained with sodium borohydride (NaBH₄) treated Halo ligand TMR (Promega)⁴⁵. Briefly, 1 μL of 1 mM Halo ligand TMR dye was reduced for 10 min in 200 μL of 50 mM sodium borohydride solution (pre-dissolved in PBS for 10 min, pH 7.4). Next, 200 μL of the reduced TMR was diluted with 800 μL of phenol-red-free DMEM to produce 1 mL reduced-TMR media. Media from transfected cells was replaced by the reduced-TMR media and cells were then placed in an incubator (5% CO₂, 37° C.) for 30 min for staining. The cells were then washed 3 times with phenol-red-free DMEM. Between washes, cells were incubated for 5 min in an incubator (5% CO₂, 37° C.).

The cells were imaged using a custom-built widefield fluorescence microscope based on an inclined illumination (HILO) scheme^{17,66}. The imaging field-of-view was set to 256×256 pixels² (33.3×33.3 μm²), and the camera integration time was set to 30 msec. The cells were imaged with a 7.7 mW 561 nm laser at an imaging rate of 43.8 msec per image for a total of 10,000 timepoints (7.3 min). During imaging, a 6.2 mW 405 nm laser was pulsed on for 50 msec once every 10 sec to photoactivate the Halo-TMR reduced ligand. Single molecules were tracked using the Fiji plugin TrackMate⁶⁸. To ensure tracks represent FB-Halo bound to 1×HA-mCh-H2B, tracks were further filtered in Mathematica. The filter eliminated tracks of length less than 16 frames. Further, all jumps between frames had to be less than 220 nm. This criteria has been used by others to distinguish transcription factors that are chromatin-bound from those that are unbound⁴⁶. Finally, in Mathematica, tracks were color-coded either according to the time at which they were acquired (as in Supplementary FIG. 5) or the average jump size between frames (as in FIG. 5).

Methods—Puromycin Treatment:

U2OS cells transiently transfected with smHA-KDM5B and FB or bead loaded with smHA-KDM5B, FB and MCP-HaloTag were imaged as above with 10 s intervals between frames. After acquiring 5 or 10 timepoints as pre-treatment images, cells were treated with a final concentration of 0.1 mg mL⁻¹ puromycin right before acquiring the 6^{th} or 11^{th} time point. After puromycin was added, the cells were imaged under the same conditions used for the pre-treatment imaging until the translation spots disappeared.

Methods—Neuron Culture and Transfection:

Rat cortical neurons were obtained from the discarded cortices of embryonic day (E)18 fetuses which were previously dissected to obtain the hippocampus, and frozen in Neurobasal medium (ThermoFisher Scientific) containing 10% fetal bovine serum (FBS, Atlas Biologicals) and 10% DimethylSulfoxide (Sigma-Aldrich, D8418) in liquid nitrogen. Cryopreserved rat cortical neurons were plated at a density of ˜15,000-30,000 cells cm⁻² on MatTek dishes (MatTek) and cultured in Neurobasal medium containing 2% B27 supplement (ThermoFisher Scientific), 2 mM L-Alanine/L-Glutamine and 1% FBS (Atlas Biologicals). Transfections were performed after 5-7 days in culture by using Lipofectamine 2000 (ThermoFisher Scientific) according to the manufacturer's instructions. Neurons co-expressing 4×HA-mRuby-Kv2.1 and FB-GFP were imaged 1-2 days post-transfection (FIG. 2B). Neurons co-expressing smHA-Kv2.1 and FB-GFP were imaged 1-7 days post-transfection (FIG. 11A). For translation assays, neurons were imaged 4-12 h post-transfection. All neuron imaging experiments were carried out in a temperature-controlled (37° C.), humidified, 5% CO₂ environment in Neurobasal medium without phenol red (ThermoFisher Scientific). Neuronal identity was confirmed by following processes emanating from the cell body to be imaged for hundreds of microns to ensure they were true neurites.

Methods—Monitoring Zebrafish Development:

All zebrafish experiments have been approved by the Tokyo Tech Genetic Experiment Safety Committee (I2018001) and animal handling is operated according to the guidelines. To visualize FB-GFP in zebrafish embryo, mRNAs for FB-GFP and N×HA-mCh-H2B were prepared. DNA fragments coding FB-GFP and N×HA-mCh-H2B were inserted into a plasmid containing the T7 promoter and poly A⁶⁹. The subsequent plasmids (T7-FB-GFP and T7-N×HA-mCh-H2B) were linearized with the XbaI restriction site for in vitro transcription using mMESSAGE mMACHINE kit (ThermoFisher Scientific). RNA was purified using RNeasy Mini Elute Cleanup Kit (QIAGEN) and resuspended in water. Before microinjection, zebrafish (AB) eggs were dechorionated by soaking in 2 mg mL⁻¹ pronase (Sigma Aldrich; P5147) in 0.03% sea salt for 10 minutes. A mixture (˜0.5 nL) containing mRNA (200 pg each for FB-GFP and N×HA-mCh-H2B) was injected into the yolk (near the cell part) of 1-cell stage embryos. For a negative control, HA-mCh-H2B mRNA was omitted. 5-10 minutes after mRNA injection, Cy5-labeled Fab specific to endogenous histone H3 Lys9 acetylation (CMA310)²⁵ was injected (100 pg in ˜0.5 nL). Injected embryos were incubated at 28° C. until the 4-cell stage and embedded in 0.5% agarose (Sigma Aldrich, A0701) in 0.03% sea salt with the animal pole down on a 35-mm glass bottom dish (MatTek). The fluorescence images were collected using a confocal microscope (Olympus; FV1000) equipped with a heated stage (Tokai Hit) set at 28° C. and a UPLSAPO 30× silicone oil immersion lens (NA 1.05), operated by the built-in FV1000 software FLUOVIEW ver.4.2. Three color images were sequentially acquired every 5 minutes using 488-, 543-, and 633-nm lasers (640×640 pixels; 0.662 μm pixel⁻¹, pinhole 800 μm; 2.0 μs pixel⁻¹) without averaging. Maximum intensity projections were created from 20 z-stacks with 5 μm.

Nuclei within zebrafish embryos were tracked in 4D using the Fiji plugin TrackMate⁶⁸. Results were post-processed and plotted with Mathematica. To quantify the number and area of nuclei and the average nuclear, cytoplasmic, and nuclear:cytoplasmic intensity through time, the intensity of all nuclei in maximum intensity projections was measured using the built-in Mathematica function ComponentMeasurements. ComponentMeasurements requires binary masks of the objects to be measured. Binary masks of the nuclei were made using the built-in Mathematica function Binarize with an appropriate intensity threshold to highlight just nuclei in images from Cy5-labeled Fab (specific to endogenous histone H3 Lys9 acetylation). Masks of the cytoplasm around each nuclei were made by dilating the nuclear masks by 4 pixels (using the built-in command Dilation) and then subtracting from the dilated mask the original nuclear masks dilated by 1 pixel. This creates ring-like masks around each nuclei, from which the average cytoplasmic intensity was measured.

Methods—Surface Plasmon Resonance:

Binding kinetics of purified FB to the HA epitope tag was measured by surface plasmon resonance (OpenSPR, Nicoyalife). After biotin-labeled HA peptide was captured by a Streptavidin sensor chip (Nicoyalife), diluted purified FB-GFP in PBS running buffer, pH 7.4, was slowly flowed over the sensor chip for 5 min to allow interaction. The running buffer was then allowed to flow for 10 min to collect the dissociation data. The non-specific binding curve was obtained by flowing the same concentration of FB-GFP in the same running buffer over a different Streptavidin sensor chip (Nicoyalife). The data from the control was collected exactly as in the experiment. For 100 nM and 30 nM of FB-GFP, the binding response was significantly higher than the control, with negligible non-specific binding interaction. Therefore, those two concentrations were chosen for binding kinetics fitting. After subtracting the control, the signal response vs time curve was obtained, as shown in FIG. 13. Binding kinetic parameters were obtained by fitting the curve to a one-to-one binding model using TraceDrawer (Nicoyalife) software (FIG. 13).

### Example 2

This example demonstrates that additional, functional scFv's can be accurately predicted and designed from sequence information.

A large number of antibody sequences (>2,000) were obtained from the Protein Data Bank (PDB). This list of sequences was combined with additional, unpublished antibody sequences generated by the inventors. For each antibody, the amino acid sequence of the heavy chain variable region (VH) and the light chain variable region (VL) was identified. Then, within each variable region, the framework regions and hypervariable regions were identified. Hypervariable regions (CDRs) were identified according to the Kabat numbering scheme. The sequence similarity of each antibody's framework regions to the framework regions of the 15F11 antibody was determined using a BLOSUM62 scoring matrix, and the antibodies were rank ordered based on the sequence similarity. Table A shows the top 150 antibodies ranked in this manner.

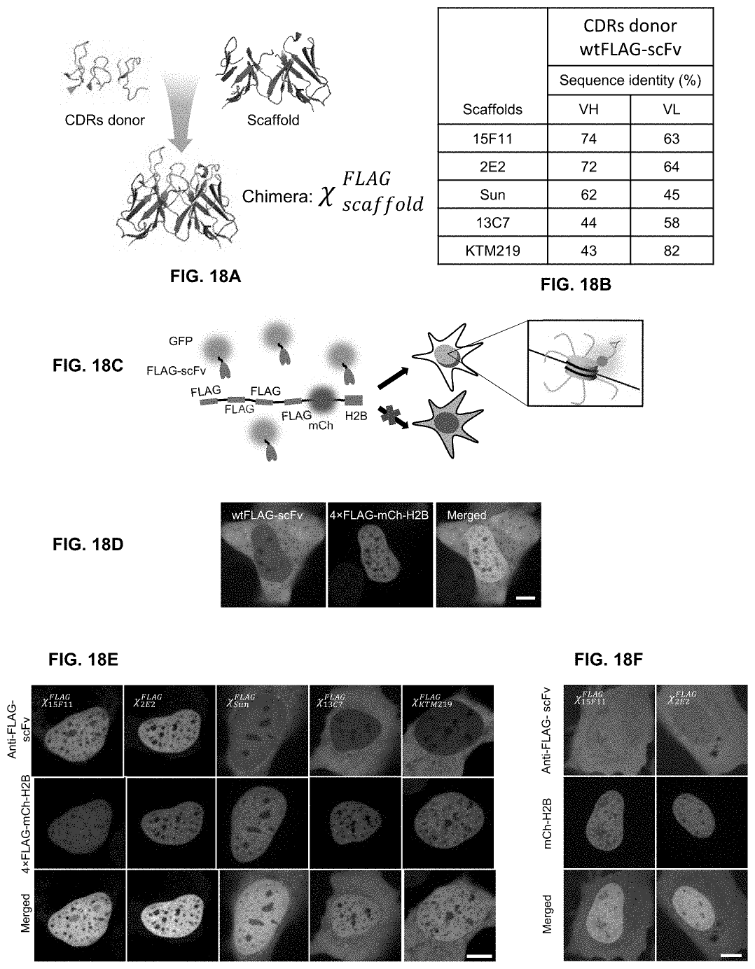

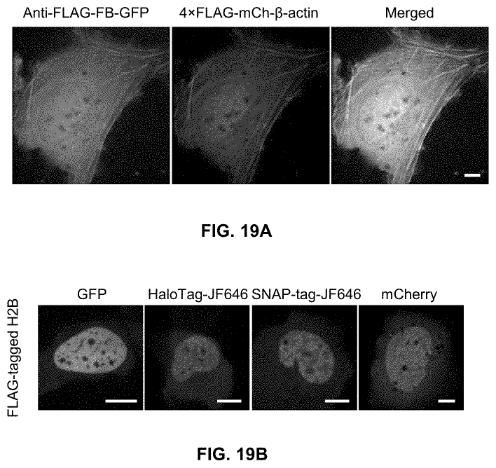

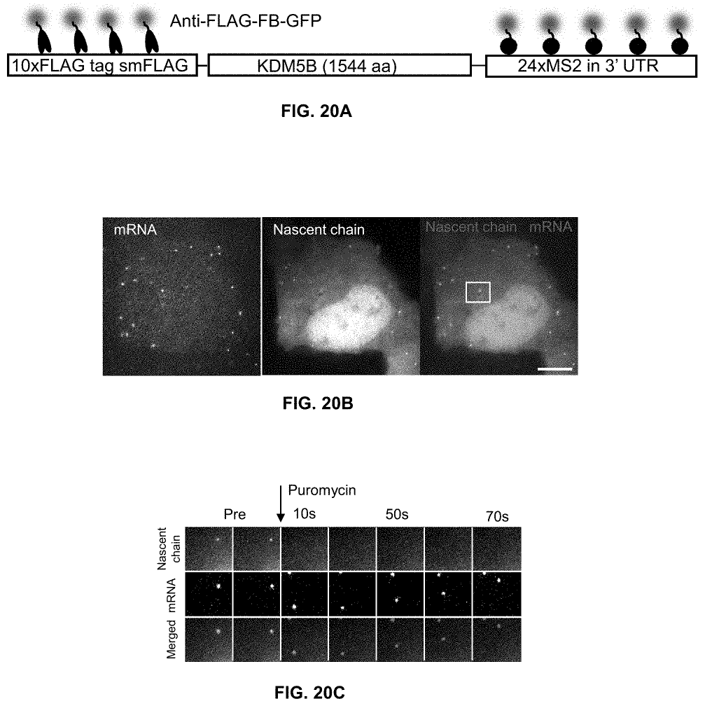

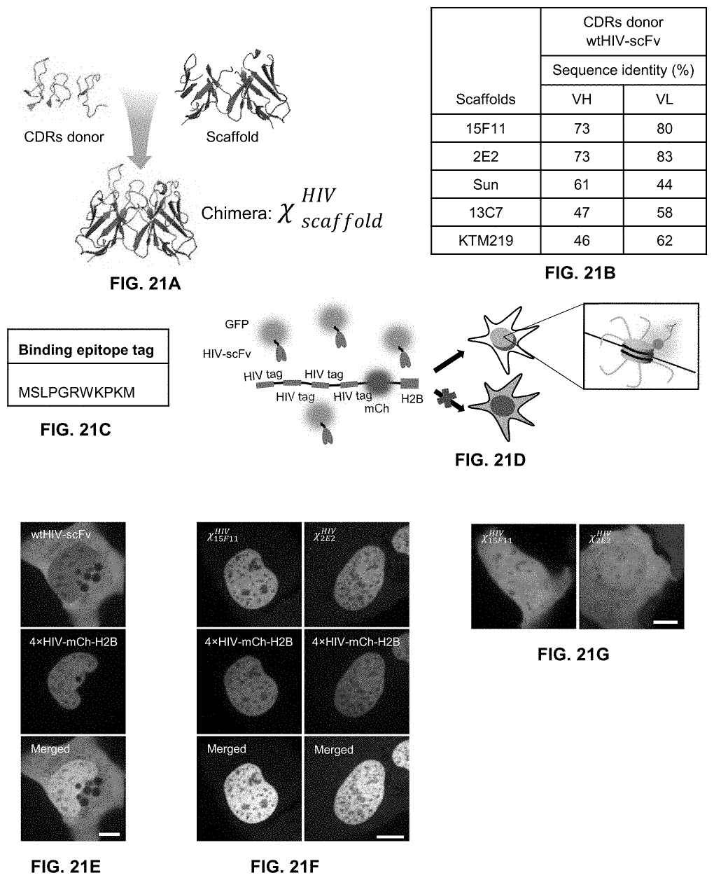

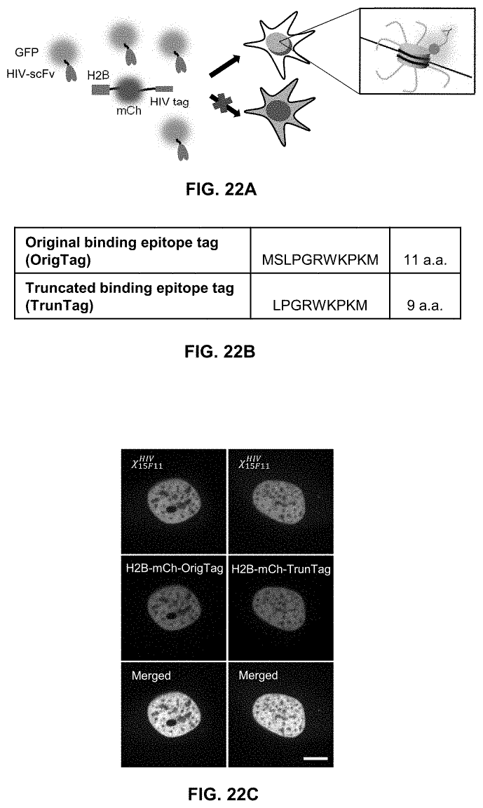

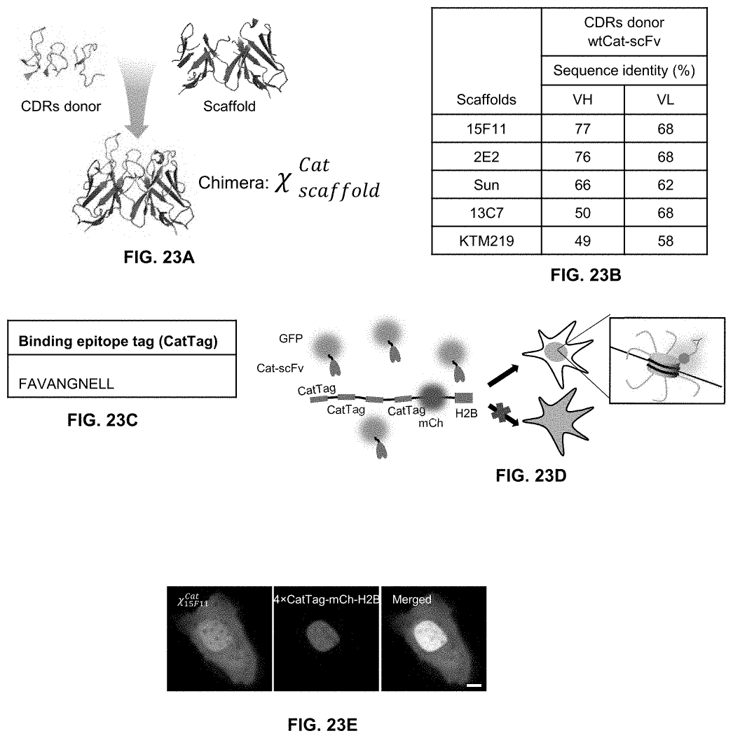

From this list, 4 antibodies were selected, and sequence alignments of each antibody's VH and VL were performed against the VH and VL of the scaffolds tested in Example 1 (Table 1). Sequence alignments of each antibody's VH and VL framework regions were also performed against the VH and VL framework regions, respectively, of the scaffolds tested in Example 1 (Table 2). Chimeric scFv's χ_{15F11}^{FLAG}, χ_{2E2}^{FLAG}, χ_{Sun}^{FLAG}, χ_{13C7}^{HFLAgA}, χ_{KTM219}^{HA}, χ_{15F11}^{HIV}, χ_{2E2}^{HIV}, χ_{15F11}^{Cat}, and χ_{2E2}^{Cat} were generated as described in Example 1. Chimeric scFv's with a 15F11 backbone or a 2E2 backbone selectively bound to target epitopes in live cells, while chimeric scFv's with a Sun, 13C7, or CTM219 backbone did not (FIG. 17-FIG. 23). These examples demonstrated the ability of the 15F11 and 2E2 backbones to support different target epitopes.

TABLE 1
Sequence identity - variable domains

| | | Donor antibodies | | | | | | | |
|---|---|---|---|---|---|---|---|---|---|
| | | wtHA-scFv | | | | wtHIV-scFv | | wtCat-scFv | |
| | | (PDB ID: 5XCS) | | wtFLAG-scFv | | (PDB ID: 2HRP) | | (PDB ID: 5VYF) | |
| | | VH | VL | VH | VL | VH | VL | VH | VL |
| SCAFFOLDS | 15F11 | 85% | 67% | 74% | 63% | 73% | 80% | 77% | 68% |
| | 2E2 | 89% | 65% | 72% | 64% | 73% | 83% | 76% | 68% |
| | 13C7 | 47% | 57% | 44% | 58% | 47% | 58% | 50% | 68% |
| | KTM219 | 47% | 65% | 42% | 82% | 46% | 62% | 49% | 58% |
| | Sun | 63% | 50% | 62% | 45% | 61% | 44% | 66% | 62% |

TABLE 2
Sequence identity - framework regions only

| | | Donor antibodies | | | | | | | |
|---|---|---|---|---|---|---|---|---|---|
| | | wtHA-scFv | | | | wtHIV-scFv | | wtCat-scFv | |
| | | (PDB ID: 5XCS) | | wtFLAG-scFv | | (PDB ID: 2HRP) | | (PDB ID: 5VYF) | |
| | | VH* | VL* | VH* | VL* | VH* | VL* | VH* | VL* |
| SCAFFOLDS | 15F11 | 94% | 75% | 84% | 72% | 85% | 85% | 84% | 71% |
| | 2E2 | 98% | 72% | 81% | 73% | 85% | 87% | 84% | 72% |
| | 13C7 | 55% | 66% | 51% | 66% | 53% | 68% | 56% | 77% |
| | KTM219 | 56% | 72% | 51% | 89% | 53% | 73% | 58% | 67% |
| | Sun | 71% | 61% | 72% | 53% | 72% | 56% | 74% | 78% |

*Framework regions only (i.e., CDRs were removed for the comparison)

TABLE 3
Sequence similarity score (BLOSUM62)

| | | Donor antibodies | | | | | | | |
|---|---|---|---|---|---|---|---|---|---|
| | | wtHA-scFv | | | | wtHIV-scFv | | wtCat-scFv | |
| | | (PDB ID: 5XCS) | | wtFLAG-scFv | | (PDB ID: 2HRP) | | (PDB ID: 5VYF) | |
| | | VH* | VL* | VH* | VL* | VH* | VL* | VH* | VL* |
| SCAFFOLDS | 15F11 | 429 | 328 | 384 | 308 | 393 | 371 | 393 | 315 |
| | 2E2 | 442 | 319 | 373 | 314 | 390 | 381 | 388 | 321 |
| | 13C7 | 282 | 306 | 257 | 298 | 269 | 302 | 288 | 332 |
| | KTM219 | 279 | 314 | 252 | 380 | 261 | 300 | 290 | 299 |
| | Sun | 344 | 247 | 350 | 216 | 346 | 224 | 368 | 306 |

*Framework regions only (i.e., CDRs were removed for the comparison)

TABLE 4
Exemplary Expression Constructs

| Plasmids (Addgene Plasmid #) | Cloning strategy |
|---|---|
| pCMV-15F11-HA-mEGFP | Gibson assembly gblock |
| pCMV-2E2-HA-mEGFP | Gibson assembly gblock |
| pCMV-13C7-HA-mEGFP | Gibson assembly gblock |
| pCMV-KIM219-HA-mEGFP | Gibson assembly gblock |
| pCMV-Suntag-HA-mEGFP | Gibson assembly gblock |
| pCMV-15F11-HA-Halo (129592) | Gibson assembly gblock |
| pCMV-15F11-HA-SNAP | Gibson assembly gblock |
| pCMV-15F11-HA-mCherry | Gibson assembly gblock |
| pCMV-4HA-mCherry-H2B | Gibson assembly gblock |
| pCMV-4HA-mCherry-Beta-Actin | Gibson assembly gblock |
| pUB-smHA-H2B-MS2 | |
| pUB-smHA-KDM5B-MS2 | (Morisaki et al. 2016b) |
| pET23b-15F11-HA-mEGFP | PCR, restriction and ligation into |
| | pET23b vector |
| pUB-smHA-Kv2.1 | PCR, restriction and ligation into |
| | pUB-smHA-KDM5B-MS2 vector |

TABLE 5
Sequence Listing

| No. | Comment | Sequence |
|---|---|---|
| 1 | Heavy chain | MAEVXLVESGGXLVKPGGSLKLSCAASGFTFS |
| | FR1 | X at position 5 can be Lys or Gln |
| | | X at position 12 can be Gly or Asp |
| 2 | Heavy chain | WVRQTPXKRLEWVA |
| | FR2 | X at position 7 can be Asp or Glu |
| 3 | Heavy chain | RFTISRDNAKNTLYLQMSSLXSEDTAXYYCAR |
| | FR3 | X at position 21 can be Arg or Lys |
| | | X at position 27 can be Ile or Met |
| 4 | Heavy chain | WGQGTXXTV |
| | FR4 | X at position 6 can be Ser or Thr |
| | | X at position 7 can be Leu or Val |
| 5 | Light chain | DIVLTQSPASLXVSLGQRATISC |
| | FR1 | X at position 12 can be Ala or Thr |
| 6 | Light chain | WYQQKPGQPPKLLIY |
| | FR2 | |
| 7 | Light chain | GIPARFSGSGSGTDFTLNIHPVEEEDAATYYC |
| | FR3 | |
| 8 | Light chain | FGXGTKLEI |
| | FR4 | X at position 3 can be Ala or Gly |
| 9 | 15F11 VH | MAEVKLVESGGGLVKPGGSLKLSCAASGFTFSSYAMSWVRQTPEKRLEWVATISSG |
| | | GSYTYYPNTVKGRFTISRDNAKNTLYLQMSSLRSEDTAIYYCARHGVRHRVDYFDY |
| | | WGQGTTLTVS |
| 10 | 15F11 VL | DIVLTQSPASLTVSLGQRATISCKASQSVDYDGDSYMNWYQQKPGQPPKLLIYAAS |
| | | NLESGIPARFSGSGSGTDFTLNIHPVEEEDAATYYCQQSNEDPLTFGAGTKLEI |
| 11 | 15F11-HA-FB | MAEVKLVESGGGLVKPGGSLKLSCAASGFTFSSYGMSWVRQTPEKRLEWVATISRG |
| | | GSYTYYPDSVKGRFTISRDNAKNTLYLQMSSLRSEDTAIYYCARRETYDEKGFAYW |
| | | GQGTTLTVSSGGGGSGGGGSGGGGSDIVLTQSPASLTVSLGQRATISCKSSQSLLN |
| | | SGNQKNYLTWYQQKPGQPPKLLIYWASTRESGIPARFSGSGSGTDFTLNIHPVEEE |
| | | DAATYYCQNDNSHPLTFGAGTKLEI |
| 12 | 2E2-HA-FB | MAEVQLVESGGDLVKPGGSLKLSCAASGFTFSSYGMSWVRQTPDKRLEWVATISRG |
| | | GSYTYYPDSVKGRFTISRDNAKNTLYLQMSSLKSEDTAMYYCARRETYDEKGFAYW |
| | | GQGTSVTVSSGGGGSGGGGSGGGGSDIVLTQSPASLAVSLGQRATISCKSSQSLLN |
| | | SGNQKNYLTWYQQKPGQPPKLLIYWASTRESGIPARFSGSGSGTDFTLNIHPVEEE |
| | | DAATYYCQNDNSHPLTFGGGTKLEI |
| 13 | 15F11-FLAG-FB | MAEVKLVESGGGLVKPGGSLKLSCAASGFTFSSFGMHWVRQTPEKRLEWVAYISSG |
| | | SSTIYYADTVKGRFTISRDNAKNTLYLQMSSLRSEDTAIYYCARSLATAAFAYWGQ |
| | | GTTLTVSSGGGGSGGGGSGGGGSDIVLTQSPASLTVSLGQRATISCRSSQSIVYSN |
| | | GNTYLEWYQQKPGQPPKLLIYKVSNRFSGIPARFSGSGSGTDFTLNIHPVEEEDAA |
| | | TYYCFQGSHVPYTFGAGTKLEI |
| 14 | 2E2-FLAG-FB | MAEVQLVESGGDLVKPGGSLKLSCAASGFTFSSFGMHWVRQTPDKRLEWVAYISSG |
| | | SSTIYYADTVKGRFTISRDNAKNTLYLQMSSLKSEDTAMYYCARSLATAAFAYWGQ |
| | | GTSVTVSSGGGGSGGGGSGGGGSDIVLTQSPASLAVSLGQRATISCRSSQSIVYSN |
| | | GNTYLEWYQQKPGQPPKLLIYKVSNRFSGIPARFSGSGSGTDFTLNIHPVEEEDAA |
| | | TYYCFQGSHVPYTFGGGTKLEI |
| 15 | 15F11-HIV-FB | MAEVKLVESGGGLVKPGGSLKLSCAASGFTFSRFGMHWVRQTPEKRLEWVAYISSG |
| | | SSTIYYADTVKGRFTISRDNAKNTLYLQMSSLRSEDTAIYYCARSGGIERYDGTYY |
| | | VMDYWGQGTTLTVSSGGGGSGGGGSGGGGSDIVLTQSPASLTVSLGQRATISCRAS |
| | | ESVDYYGKSFMNWYQQKPGQPPKLLIYAASNQGSGIPARFSGSGSGTDFTLNIHPV |
| | | EEEDAATYYCQQSKEVPWTFGAGTKLEI |
| 16 | 2E2-HIV-FB | MAEVQLVESGGDLVKPGGSLKLSCAASGFTFSRFGMHWVRQTPDKRLEWVAYISSG |
| | | SSTIYYADTVKGRFTISRDNAKNTLYLQMSSLKSEDTAMYYCARSGGIERYDGTYY |
| | | VMDYWGQGTSVTVSSGGGGSGGGGSGGGGSDIVLTQSPASLAVSLGQRATISCRAS |
| | | ESVDYYGKSFMNWYQQKPGQPPKLLIYAASNQGSGIPARFSGSGSGTDFTLNIHPV |
| | | EEEDAATYYCQQSKEVPWTFGGGTKLEI |
| 17 | 15F11-Cat-FB | MAEVKLVESGGGLVKPGGSLKLSCAASGFTFSSYAMSWVRQTPEKRLEWVAAISGR |
| | | GYNADYADSVKGRFTISRDNAKNTLYLQMSSLRSEDTAIYYCARLEYFDYWGQGTT |
| | | LTVSSGGGGSGGGGSGGGGSDIVLTQSPASLTVSLGQRATISCRASQSISSWLAWY |
| | | QQKPGQPPKLLIYKASSLESGIPARFSGSGSGTDFTLNIHPVEEEDAATYYCQQYN |
| | | SYPLTFGAGTKLEI |
| 18 | Linker | GGGGS |
| 19 | Linker | GGGGSGGGGSGGGGS |
| 20 | Linker | SSGGGGSGGGGSGGGGS |
| 21 | Epitope tag | MSLPGRWKPKM |
| | for wtHIV- | |
| | scFV | |
| 22 | Truncated | LPGRWKPKM |
| | epitope tag | |
| | for wtHIV- | |
| | scFV | |
| 23 | HA epitope | YPYDVPDYA |
| 24 | Sun-Tag | EELLSKNYHLENEVARLKK |
| | epitope | |
| 25 | 15F11 HC FR1 | MAEVKLVESGGGLVKPGGSLKLSCAASGFTFS |
| 26 | 15F11 HC FR2 | WVRQTPEKRLEWVA |
| 27 | 15F11 HC FR3 | RFTISRDNAKNTLYLQMSSLRSEDTAIYYCAR |
| 28 | 15F11 HC FR4 | WGQGTTLTV |
| 29 | 15F11 LC FR1 | DIVLTQSPASLTVSLGQRATISC |
| 30 | 15F11 LC FR2 | WYQQKPGQPPKLLIY |
| 31 | 15F11 LC FR3 | GIPARFSGSGSGTDFTLNIHPVEEEDAATYYC |
| 32 | 15F11 LC FR4 | FGAGTKLEI |
| 33 | 2E2-Cat-FB | MAEVKQVESGGDLVKPGGSLKLSCAASGFTFSSYAMSWVRQTPDKRLEWVAAISGR |
| | | GYNADYADSVKGRFTISRDNAKNTLYLQMSSLKSEDTAMYYCARLEYFDYWGQGTS |
| | | VTVSSGGGGSGGGGSGGGGSDIVLTQSPASLAVSLGQRATISCRASQSISSWLAWY |
| | | QQKPGQPPKLLIYKASSLESGIPARFSGSGSGTDFTLNIHPVEEEDAATYYCQQYN |
| | | SYPLTFGGGTKLEI |
| 34 | NLS | PKKKRKV |
| 35 | NLS | PKKKRRV |
| 36 | NLS | KRPAATKKAGQAKKKK |
| 37 | TAT cell- | GRKKRRQRRRPPQPKKKRKV |
| | penetrating | |
| | peptide | |
| 38 | TLM cell- | PLSSIFSRIGDPPKKKRKV |
| | penetrating | |
| | peptide | |
| 39 | MPG cell- | GALFLGWLGAAGSTMGAPKKKRKV |
| | penetrating | |
| | peptide | |
| 40 | MPG cell- | GALFLGFLGAAGSTMGAWSQPKKKRKV |
| | penetrating | |
| | peptide | |
| 41 | Pep-1 cell- | KETWWETWWTEWSQPKKKRKV |
| | penetrating | |
| | peptide | |
| 42 | FLAG VH CDR1 | SFGMH |
| 43 | FLAG VH CDR2 | YISSGSSTIYYADTVKG |
| 44 | FLAG VH CDR3 | SLATAAFAY |
| 45 | FLAG VL CDR1 | RSSQSIVYSNGNTYLE |
| 46 | FLAG VL CDR2 | KVSNRFS |
| 47 | FLAG VL CDR3 | FQGSHVPYT |
| 48 | CatTag | FAVANGNELL |
| | epitope | |
| 49 | FLAG epitope | DYKDDDDK |

1. A single chain variant fragment (scFv) comprising a heavy chain variable domain (VH) of Formula (I), a light chain variable domain (VL) of Formula (I), and a linker connecting VH and VL,
FR1-HVR1-FR2-HVR2-FR3-HVR3-FR4 (I),
wherein FR is framework region, HVR is hypervariable region, and - is a peptide bond in each instance; and wherein
the amino acid sequence of FR1, FR2, FR3 and FR4, collectively, for the VH has at least 80% identity to the amino acid sequence of the framework regions of SEQ ID NO: 9;
the amino acid sequence of FR1, FR2, FR3 and FR4, collectively, for the VL has at least 65% identity to the amino acid sequence of the framework regions of SEQ ID NO: 10; and
the scFv has a different antigen-binding specificity than 15F11.

2-5. (canceled)

6. The single chain variant fragment of claim 1, wherein the VH of the scFV has at least about 74% identity to SEQ ID NO: 9.

7. The single chain variant fragment of claim 6, wherein the VL of the scFV has at least about 63% identity to SEQ ID NO: 10.

8. The single chain variant fragment of claim 1, wherein the linker is a peptide linker.

9. The single chain variant fragment of claim 8, wherein the peptide linker consists of about 5 to about 30 amino acids.

10. (canceled)

11. The single chain variant fragment of claim 8, wherein the peptide linker comprises (GGGGS)ₙ, wherein n is 1 to 6.

12. (canceled)

13. The single chain variant fragment of claim 1, wherein the scFv specifically binds SEQ ID NO: 21, SEQ ID NO: 22, SEQ ID NO: 23, SEQ ID NO: 24, SEQ ID NO: 48 or SEQ ID NO: 49.

14. The single chain variant fragment of claim 1, wherein the scFv further comprises HVRs derived from the antibody F11.2.32, the antibody REGN1909, or the antibody 12CA5.

15. The single chain variant fragment of claim 1, wherein the scFv further comprises HVRs derived from SEQ ID NOs: 42, 43, 44, 45, 46 and 47.

16. The single chain variant fragment of claim 1, wherein the scFv further comprises HVRs derived from an antibody listed in Table A.

17. A protein comprising an scFv of claim 1.

18. The protein of claim 17, wherein the protein further comprises a fluorescent protein, a bioluminescent protein, a protein of interest, a self-labeling tag, a toxin, a drug, or any combination thereof, and an optional linker.

19. The protein of claim 18, wherein the protein comprises a peptide linker.

20-21. (canceled)

22. The protein of claim 19, wherein the peptide linker comprises (GGGGS)ₙ, wherein n is 1 to 6.

23-24. (canceled)

25. The protein of claim 17, wherein the protein further comprises a sub-cellular localization signal or a cell penetrating domain.

26-27. (canceled)

28. The protein of claim 17, wherein the protein further comprises an affinity tag at either the N-terminus or the C-terminus of the protein and optionally a protease cleavage site at the proximal end of the affinity tag.

29. A polynucleotide encoding a single chain variant of claim 1.

30. A polynucleotide encoding a protein of claim 17.

31. (canceled)

32. A vector comprising the polynucleotide of claim 29.

33. A method for live cell imaging, the method comprising providing a protein of claim 1, and a cell comprising an epitope to which the scFv specifically binds; labeling the cell with the protein; and imaging the cell to detect and optionally quantify the protein.

34. (canceled)

35. The method of claim 33, wherein the cell stably or transiently expresses a fusion protein comprising the epitope to which the scFv specifically binds

36. (canceled)

37. The method of claim 35, wherein the fusion protein comprises two or more copies of the epitope to which the scFv specifically binds.

38. The method of claim 35 wherein the epitope comprises SEQ ID NO: 22, SEQ ID NO: 23, SEQ ID NO: 24, SEQ ID NO: 48, or SEQ ID NO: 49.

39. A vector comprising the polynucleotide of claim 30.
